(19) United States
(12) Patent Application Publication
(10) Pub. No.: US 2017/0072076 A1
(43) Pub. Date: Mar. 16, 2017

(54) CARRIERS FOR PROCESSING POUCHES AND OTHER IRREGULAR CONTAINERS AND OBJECTS

(71) Applicant: John Bean Technologies Corporation, Chicago, IL (US)

(72) Inventors: Zhijun Weng, Fresno, CA (US); Jeffrey A. Dahl, Fresno, CA (US); Frederick James Wilson, Fresno, CA (US); Fred S. Guerrero, Madera, CA (US); Terry L. Heyliger, Truckee, CA (US); William C. Kreamer, Truckee, CA (US); Kevin Andrew Carlson, Kerman, CA (US); Jenaro Morris Martinez, Fresno, CA (US); Walter Kenneth Mizuno, Reedley, CA (US); Stacy Michelle Allen, Fresno, CA (US); Joshua Logan Fender, Fresno, CA (US); Andrew C. Prins, Chowchilla, CA (US); Jacob Lee Richardson, Clovis, CA (US)

(73) Assignee: John Bean Technologies Corporation, Chicago, IL (US)

(21) Appl. No.: 15/269,884

(22) Filed: Sep. 19, 2016

Related U.S. Application Data
(63) Continuation-in-part of application No. 13/554,969, filed on Jul. 20, 2012.
Application No. 13/554,969 is a continuation-in-part of application No. 14/051,745, filed on Oct. 11, 2013, now abandoned.
Application No. 14/051,745 is a continuation-in-part of application No. 13/554,969, filed on Jul. 20, 2012.
(60) Provisional application No. 61/566,524, filed on Dec. 2, 2011.
(60) Provisional application No. 61/510,419, filed on Jul. 21, 2011.
(60) Provisional application No. 61/566,524, filed on Dec. 2, 2011.
(60) Provisional application No. 61/510,419, filed on Jul. 21, 2011.

(51) Int. Cl. A61L 2/04

(52) U.S. Cl. CPC A61L 2/04

(57) ABSTRACT

A carrier for processing a container in a thermal processing system includes a carrier body having an exterior configured to roll in the thermal processing system. The container includes a container body extending between first and second container ends. The carrier body has a first end portion configured to receive the first container end and a second end portion opposite the first end portion. A retention system is disposed within the second end portion of the carrier body that is configured to engage the second container end and retain the container within the body.

### CROSS-REFERENCES TO RELATED APPLICATIONS

This application is a continuation-in-part of U.S. patent application Ser. No. 13/554,969, filed Jul. 20, 2012, which claims the benefit of U.S. Provisional Application No. 61/566,524, filed Dec. 2, 2011, and 61/510,419, filed Jul. 21, 2011; and is also a continuation-in-part of U.S. patent application Ser. No. 14/051,745, filed Oct. 11, 2013, which claims the benefit of U.S. Provisional Application No. 61/566,524, filed Dec. 2, 2011, and 61/510,419, filed Jul. 21, 2011, the disclosures of all of which are hereby expressly incorporated by reference in their entirety herein.

### BACKGROUND

In food processing, various types of continuous sterilization apparatuses have been used for sterilizing canned foods and beverages. While cans are still very popular for containing and processing foods, pouches and other types of containers are also becoming more widely used for food processing. For some food products, pouches are preferred because they are easier to open and easier to dispose of than cans. However, continuous processes, such as continuous rotary sterilization processes, are designed for use with cylindrical containers such as cans and not for pouches.

Carriers designed for use with continuous sterilization apparatuses that are capable of holding irregularly shaped and/or fragile containers, such as bottles, flat pouches, and other containers have been developed in the past. See, for example, U.S. Pat. Nos. 3,314,560, 4,385,035, and 5,245,916. Although effective, these carriers require assembly of multiple parts, adding complexity to the overall process. Because these carriers have not been optimized for ease of use, there exists a need for optimized carriers for processing irregularly shaped and/or fragile containers, including, but not limited to, pouches, thin-walled cans, bottles, tubs, and other containers.

It has been common to sterilize canned foods and beverages in a rotary sterilizer, for example, using a sterilizer of the type shown in FIGS. 18 and 19. The illustrated sterilizer 1200 includes an agitating reel 1202. An exterior rotary valve 1204 feeds the reel 1202 of the rotary sterilizer 1200 with containers in the form of cans 1206. The reel 1202 is surrounded by an outer shell 1208. The cans 1206 are rotated around the reel 1202 and are maintained in position along the reel 1202 by angle members 1210 of the reel as shown in FIG. 19, the angle members forming pockets or cells 1211 about the periphery of the reel 1202.

The rotary valve 1204 itself has a feeder wheel 1212 that receives the cans 1206 from an in-feed conveyor 1214. The feeder wheel has circumferential pockets 1216 defined by outwardly extending fins 1218 that receive the cans 1206 between two adjacent fins and move the cans through the valve 1204 into registry with the cells or pockets 1211 of the reel 1202 formed by the angle members 1210. In this manner, the cans are loaded into the rotary sterilizer at a very high rate of speed.

When the cans 1206 are at the upper portion of the reel 1202, they are supported by the reel itself, but when the cans are traveling along the bottom portion of the reel 1202, the cans are supported by the outer shell 1208, and are pushed along the outer shell by the angle members 1210 located behind the cans, as shown in FIG. 19. When supported by outer shell 1208, the cans 1206 roll along the internal surface of the outer shell 1208, and thereupon the can contents are agitated by such rolling action.

Historically, cans, such as cans 1206, have been of substantially sturdy construction, with the rims or chines at the upper or lower edges of the cans extending beyond the outer surface of the can so as not to dent or otherwise damage the can outer surface during the agitating process as the cans roll along the outer shell, or at other times when the cans are in contact with other components of the sterilizer 1200, for example, during transfer of the cans from the conveyor 1214 to the rotary valve 1204, transfer from the rotary valve 1204 to the reel 1202, movement of the cans along the sterilizer and transfer of the cans from the sterilizer to an output mechanism for further processing. Typically after the sterilization has been completed, a paper label is applied to the exterior of the can.

Food processors desire to now manufacture cans with thinner or lighter gauge material. However, the use of such lighter gauge material causes the cans to be more easily damaged during processing in the rotary sterilizer. Also, it is desirable to be able to use cans with a lithographically applied label and/or design to the can exterior prior to the sterilization of the can contents. However, such lithographic finishes can easily be scratched or otherwise damaged during the rotary sterilization process.

Moreover, it is desirable to be able to sterilize cans with necked-in tops and bottoms, for example, cans of the type used for soft drinks, in a rotary sterilizer rather than in a batch-type retort. Rotary sterilizers have much higher throughput than do batch retorts. However, necked-in cans do not have chines that extend outwardly of the exterior body of the can that serve to help protect the can exterior during the rotary sterilization process. Thus, if such cans were utilized in conjunction with the rotary sterilizer, the lithographed exterior of such cans would be scratched or otherwise damaged during the rotary sterilization process.

In addition, it is now common to package numerous types of beverages and liquid foods in plastic or other types of bottles. To date, sterilization of such bottles has been limited to using a batch retort. It would be advantageous to be able to sterilize the contents of such bottles using a rotary sterilizer for increased throughput. However, many bottles do not have the structural integrity to withstand the relatively rough handling, high acceleration and deceleration speeds that would be experienced in a rotary sterilizer. Also, some bottles are not cylindrical, and thus would not roll correctly on the rotary shell 1208.

The present disclosure seeks to address the above shortcomings with respect to the use of a rotary sterilizer with, for example, thin gauge metallic cans, necked-in cans, cans with a lithographic exterior, as well as plastic or other bottles.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In accordance with one embodiment of the present disclosure, a carrier for a container for processing the container in a rotary processing system is provided. The carrier generally includes a carrier body defining at least one compartment for receiving at least one container, wherein the carrier is capable of rolling, and a retention system for maintaining the at least one container in the at least one compartment and restricting movement of the at least one container within the at least one compartment.

In accordance with another embodiment of the present disclosure, a carrier for a container for processing the container in a rotary processing system is provided. The carrier generally includes a carrier body defining at least one compartment for receiving at least one container, and a removable outer shell for the carrier body.

In accordance with another embodiment of the present disclosure, a method of loading a carrier with a container is provided. The method generally includes obtaining a carrier defining at least one compartment for receiving at least one container and a retention system for maintaining the at least one container in the at least one compartment. The method further includes loading the at least one container in the at least one compartment, and activating the retention system to restrict the movement of the at least one container within the at least one compartment.

In accordance with another embodiment of the present disclosure, a method of loading a carrier with a container is provided. The method generally includes obtaining a carrier defining first and second compartments hingedly coupled to one another between open and closed positions and defining a cylinder when in the closed position. The method further includes placing the first and second compartments in the open positions and receiving at least one container in either of the first and second compartments, restricting the movement of the at least one container within either of the first and second compartments, and hingedly coupling the first and second compartments in the closed position to define a carrier.

In accordance with another embodiment of the present disclosure, a method of processing a container in a rotary processing system is provided. The method generally includes placing at least one container in a carrier, and receiving the at least one carrier in the rotary system.

In accordance with another embodiment of the present disclosure, a system for unloading a container from a carrier having at least one compartment for receiving at least one container and a retention system for maintaining the at least one container in the at least one compartment is provided. The system generally includes a conveyor assembly for conveying the carrier in a controlled path, and an unloading assembly for opening the at least one compartment.

In accordance with another embodiment of the present disclosure, a method for unloading a container from a carrier is provided. The method generally includes receiving on a conveyor assembly, a carrier having at least one compartment for receiving at least one container and a retention system for maintaining the at least one container in the at least one compartment, wherein the conveyor assembly is configured for conveying the carrier in a controlled path, and opening the at least one compartment.

In accordance with another embodiment of the present disclosure, a carrier for a container for processing the container in a rotary processing system includes a body for receiving the container therein. The body includes an exterior circular portion to enable the body to roll on said circular portion. The body also includes an interior portion sized to receive the container and hold the container within the interior of the body by applying a load on the container.

In accordance with another embodiment of the present invention, the carrier body interior portion is expandable to define a container receiving cavity. The body interior is expandable to receive the container therein and then contractible to apply a load against the container. In this regard, the body interior portion is composed of at least one material selected from a group consisting of a compressible material, an elastic material, a flexible material, and a deformable material.

In accordance with a further embodiment of the present disclosure, the carrier is in the form of at least one end cap assembly engageable with an end portion of a container. The at least one end cap assembly having an interior portion which defines the interior portion of the body for receiving the end portion of the container therein.

In accordance with another embodiment of the present disclosure, the at least one end cap assembly includes an outer housing with an end wall, the outer housing being substantially circular and sized and configured to function as the exterior of the carrier body. The interior of the carrier body is disclosed within the end cap assembly housing. The interior portion of the body may be in the form of a liner for receiving and retaining the container therein.

In accordance with another embodiment of the present disclosure, a carrier for a container for processing the container in a rotary processing system includes spaced apart rim portions, with the container capable of passing through at least one of the rim portions. An expandable body extends between the spaced apart rim portions and has a nominal exterior diameter that is smaller than the diameter of the rim portions. The body is expandable to receive the container therein and apply a load against the container for retaining the conveyor within the expandable body.

In accordance with the further embodiments of the present invention, the expandable body portion includes openings to enable processing fluid to reach the container.

In accordance with another aspect of the present disclosure, the expandable body includes an exterior shell and an interior liner. The interior liner is selected from a material consisting of one of a compressible material, an elastic material, a flexible material, and a deformable material.

In accordance with another aspect of the present disclosure, a carrier for processing a container in a thermal processing system includes a carrier body having an exterior configured to roll in the thermal processing system. The container includes a container body extending between first and second container ends. The carrier body has a first end portion configured to receive the first container end and a second end portion opposite the first end portion. A retention system is disposed within the second end portion of the carrier body that is configured to engage the second container end and retain the container within the body.

In another aspect, a carrier for a container for processing the container in a thermal processing system includes a body having an exterior configured to roll in a thermal processing system, the body having a first end portion configured to receive a first end of a container and a second end portion opposite the first end portion. The carrier further includes a retaining member removably received within the second end portion of the body. The retaining member is engageable with a second end of the container for retaining the container in place within the body.

In another aspect, a carrier for a container for processing the container in a thermal processing system includes a body having first and second end portions that are configured to roll in the thermal processing system. The body includes an interior configured to receive a container, wherein the interior of the body has a diameter that is substantially the same as an outer diameter of the container.

In yet another aspect, a carrier for a container for processing the container in a thermal processing system includes a body having an interior and an exterior configured to roll in a thermal processing system and a retention system disposable within the interior of the body that is configured to removably receive a container therein. The retention system includes at least one flexible finger to receive the container and hold the container within the body, wherein the at least one finger is moveable between a first position, wherein the at least one finger is spaced from the container to receive the container into the body, and a second position, wherein the at least one flexible finger holds the container within the body.

In yet another aspect, a rotary processing system includes a pocket configured to receive and move a carrier within the rotary processing system and a carrier sized to be received and retained within the pocket during movement within the rotary processing system. The carrier includes a body having first and second end portions that are configured to roll in the thermal processing system. The body also includes an interior configured to receive a container, wherein the interior of the body has a diameter that is larger than the outer diameter of the container.

### DESCRIPTION OF THE DRAWINGS

The foregoing aspects and many of the attendant advantages of this disclosure will become more readily appreciated by reference to the following detailed description, when taken in conjunction with the accompanying drawings, wherein:

FIG. 1 is a front view of a rotary sterilizer, which is in the process of rotating two pouch carriers that have been designed in accordance with one embodiment of the present disclosure;

FIG. 2 is an isometric view of the pouch carrier shown in FIG. 1, showing pouches being inserted into the pouch carrier;

FIG. 3 is an isometric view of a pouch carrier in accordance with another embodiment of the present disclosure;

FIGS. 4 and 5 are side cross-sectional views of the pouch carrier of FIG. 3, showing pouches in the pouch sleeves;

FIGS. 6A-15B are various views of various examples of sleeve retention mechanisms for pouch carriers in accordance with embodiments of the present disclosure;

FIGS. 16A-16C are top views of various examples of sleeve shapes and configurations for pouch carriers in accordance with embodiments of the present disclosure;

FIG. 17 is a top view of trapezoidal sleeve shape shown in FIG. 16A, combined with a sleeve retention mechanism, similar to that shown in FIGS. 14A and 14B, in accordance with one embodiment of the present disclosure;

FIGS. 18-20 are directed to various designs for rotary sterilizers in accordance with embodiments of the present disclosure;

FIGS. 21-23 are various views of systems for processing pouch carriers in accordance with embodiments of the present disclosure;

FIG. 24 is an isometric view of a bottle carrier in accordance with another embodiment of the present disclosure;

FIG. 25 is an isometric view of a tub carrier in accordance with another embodiment of the present disclosure;

FIG. 26 is an isometric view of a laminate container carrier in accordance with another embodiment of the present disclosure;

FIG. 27 is an exploded view of the carriers shown in FIGS. 24 and 25, showing interchangeability between carrier sleeve portions and outer shell portions;

FIGS. 28-29B are isometric view of carriers in accordance with other embodiments of the present disclosure;

FIGS. 30-38 are views of various embodiments of carriers designed for holding fragile containers, such as thin-walled cans and plastic bottles.

FIGS. 39, 40, and 41 depict a carrier in accordance with an embodiment of the present disclosure;

FIGS. 42 and 43 illustrate a container C being inserted into the carrier, as well as being held by the carrier shown in FIGS. 39, 40, and 41;

FIG. 44 depicts another carrier in accordance with the present disclosure;

FIG. 45 depicts a further carrier in accordance with the present disclosure;

FIGS. 46, 47, 48, 49 and 50 depict the further embodiment of the carrier in accordance with the present disclosure;

FIG. 51 depicts a further carrier in accordance with the present disclosure;

FIG. 52 depicts another carrier in accordance with the present disclosure;

FIG. 53 depicts a further carrier in accordance with the present disclosure;

FIG. 54 depicts another carrier in accordance with the present disclosure;

FIG. 55 depicts another carrier in accordance with the present disclosure;

FIG. 56 depicts another carrier in accordance with the present disclosure;

FIG. 57 depicts another carrier in accordance with the present disclosure;

FIG. 58 depicts a further carrier in accordance with the present disclosure;

FIG. 59 depicts a further carrier in accordance with the present disclosure;

FIG. 60 depicts another carrier in accordance with the present disclosure;

FIG. 61 depicts a further carrier in accordance with the present disclosure;

FIG. 62 depicts another carrier in accordance with the present disclosure;

FIG. 63 depicts another carrier in accordance with the present disclosure;

FIGS. 64, 65, 66, and 67 depict another carrier in accordance with the present disclosure;

FIGS. 68 and 69 depict a further carrier of the present disclosure;

FIGS. 70A and 70B depict a further carrier in accordance with the present disclosure;

FIG. 71 depicts a further carrier in accordance with the present disclosure;

FIGS. 72A and 72B depict a further carrier of the present disclosure;

FIG. 73 depicts a further carrier of the present disclosure;

FIG. 74 depicts a further carrier of the present disclosure;

FIG. 75 depicts another carrier of the present disclosure;

FIG. 76 depicts a further carrier of the present disclosure;

FIGS. 77A, 77B, and 77C depict another carrier of the present disclosure;

FIGS. 78A, 78B, and 78C depict a further carrier of the present disclosure;

FIGS. 79A, 79B, and 79C depict a further carrier of the present disclosure;

FIGS. 80A, 80B, and 80C depict a further carrier of the present disclosure;

FIGS. 81A, 81B, and 81C depict a further carrier of the present disclosure;

FIGS. 82A and 82B depict a further carrier of the present disclosure;

FIGS. 83A, 83B, and 83C depict a further carrier of the present disclosure;

FIGS. 84A, 84B, and 84C depict a further carrier of the present disclosure;

FIGS. 85A, 85B, and 85C depict a further carrier of the present disclosure;

FIG. 86 is a top isometric view of another carrier formed in accordance with the present disclosure for receiving and retaining a container C therein;

FIG. 87 is a bottom isometric view of the carrier of FIG. 86;

FIG. 88 is a top isometric exploded view of the carrier of FIG. 86, wherein the container C is not shown for clarity;

FIG. 89 is a bottom isometric exploded view of the carrier of FIG. 86, wherein the container C is not shown for clarity;

FIG. 90 is a top view of the carrier of FIG. 86;

FIG. 91 is a cross-sectional view of the carrier of FIG. 90, taken substantially across line 91-91;

FIG. 92 is a top isometric view of another carrier formed in accordance with the present disclosure for receiving and retaining a container C therein;

FIG. 93 is a bottom isometric view of the carrier of FIG. 92;

FIG. 94 is a top isometric exploded view of the carrier of FIG. 92, wherein the container C is not shown for clarity;

FIG. 95 is a bottom isometric exploded view of the carrier of FIG. 92, wherein the container C is not shown for clarity;

FIG. 96 is a top view of the carrier of FIG. 92;

FIG. 97 is a cross-sectional view of the carrier of FIG. 96, taken substantially across line 97-97;

FIG. 98 is a top isometric view of another carrier formed in accordance with the present disclosure for receiving and retaining a container C therein;

FIG. 99 is a bottom isometric view of the carrier of FIG. 98;

FIG. 100 is an isometric exploded view of the carrier of FIG. 98;

FIG. 101 is a top view of the carrier of FIG. 98;

FIG. 102 is a cross-sectional view of the carrier of FIG. 101, taken substantially across line 102-102;

FIG. 103 is a top isometric view of another carrier formed in accordance with the present disclosure for receiving and retaining a container C therein;

FIG. 104 is a bottom isometric view of the carrier of FIG. 103;

FIG. 105 is a top isometric exploded view of the carrier of FIG. 103, wherein the container C is not shown for clarity;

FIG. 106 is a bottom isometric exploded view of the carrier of FIG. 103, wherein the container C is not shown for clarity;

FIG. 107 is a top view of the carrier of FIG. 103;

FIG. 108 is a cross-sectional view of the carrier of FIG. 107, taken substantially across line 108-108;

FIG. 109 is a top isometric view of an alternative embodiment of a clip for use with a carrier formed in accordance with the present disclosure;

FIG. 110 is a bottom isometric view of the clip of FIG. 109;

FIG. 111 is a top isometric view of another alternative embodiment of a clip for use with a carrier formed in accordance with the present disclosure;

FIG. 112 is a bottom isometric view of the clip of FIG. 111;

FIG. 113 is a top isometric view of another carrier formed in accordance with the present disclosure for receiving and retaining a container C therein;

FIG. 114 is a bottom isometric view of the carrier of FIG. 113;

FIG. 115 is a top view of the carrier of FIG. 113;

FIG. 116 is a cross-sectional view of the carrier of FIG. 113, taken substantially across line 116-116; and

FIG. 117 is a partial cross-sectional view of another carrier formed in accordance with the present disclosure for receiving and retaining a container X therein.

### DETAILED DESCRIPTION

The detailed description set forth below in connection with the appended drawings, where like numerals reference like elements, is intended as a description of various embodiments of the disclosed subject matter and is not intended to represent the only embodiments. Each embodiment described in this disclosure is provided merely as an example or illustration and should not be construed as preferred or advantageous over other embodiments. The illustrative examples provided herein are not intended to be exhaustive or to limit the disclosure to the precise forms disclosed. Similarly, any steps described herein may be interchangeable with other steps, or combinations of steps, in order to achieve the same or substantially similar result.

In the following description, numerous specific details are set forth in order to provide a thorough understanding of exemplary embodiments of the present disclosure. It will be apparent to one skilled in the art, however, that many embodiments of the present disclosure may be practiced without some or all of the specific details. In some instances, well-known process steps have not been described in detail in order not to unnecessarily obscure various aspects of the present disclosure. Further, it will be appreciated that embodiments of the present disclosure may employ any combination of features described herein.

The present application may include references to directions, such as “forward,” “rearward,” “front,” “back,” “upward,” “downward,” “right-hand,” left-hand,” “in,” “out,” “extended,” “advanced,” “retracted,” “proximal,” “distal,” “central,” etc. These references and other similar references in the present application are only to assist in helping describe and understand the present invention and are not intended to limit the present invention to these directions or locations. Also, references to “container,” “can,” or similar are understood to be interchangeable and are not meant to be limiting in nature.

The present application may also reference quantities and numbers. Unless specifically stated, such quantities and numbers are not to be considered restrictive, but exemplary of the possible quantities or numbers associated with the present application. Also in this regard, the present application may use the term “plurality” to reference a quantity or number. In this regard, the term “plurality” is meant to be any number that is more than one, for example, two, three, four, five, etc. The term “about,” “approximately,” etc., means plus or minus 5% of the stated value.

Embodiments of the present disclosure are directed to carriers for irregularly shaped and/or fragile containers or other objects, such as pouches, bottles, thin-walled cans, tubs, fiberboard containers, fiberboard and plastic laminate containers (such as TETRA RECART® containers), and other objects. The various carriers and methods described herein are particularly useful in cooking and sterilization processes using rotary systems, such as rotary sterilizers. However, it should be appreciated that the carriers described herein may also be useful in other non-food-related rotary processes.

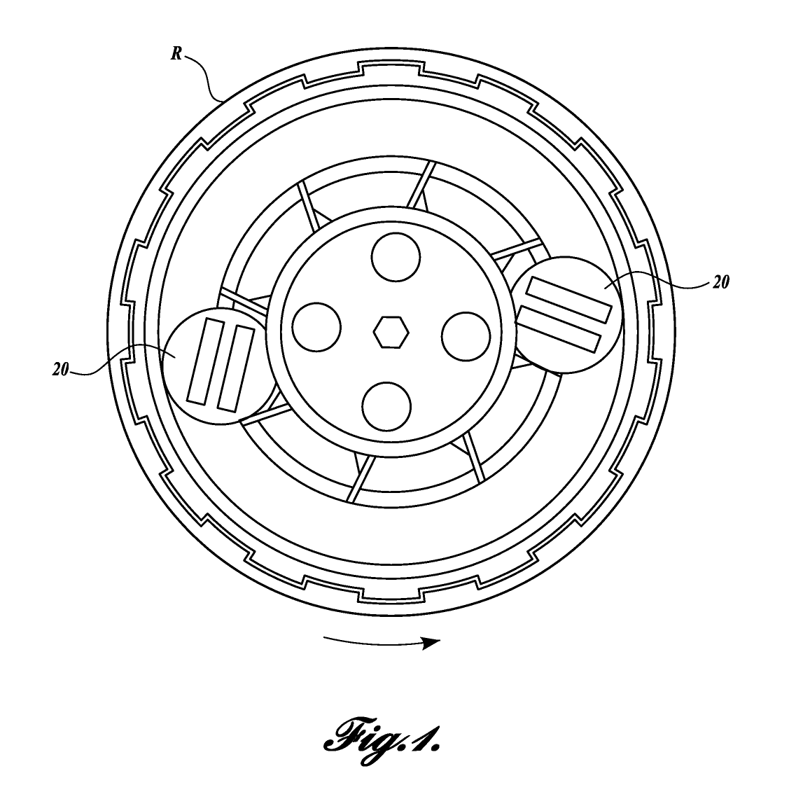

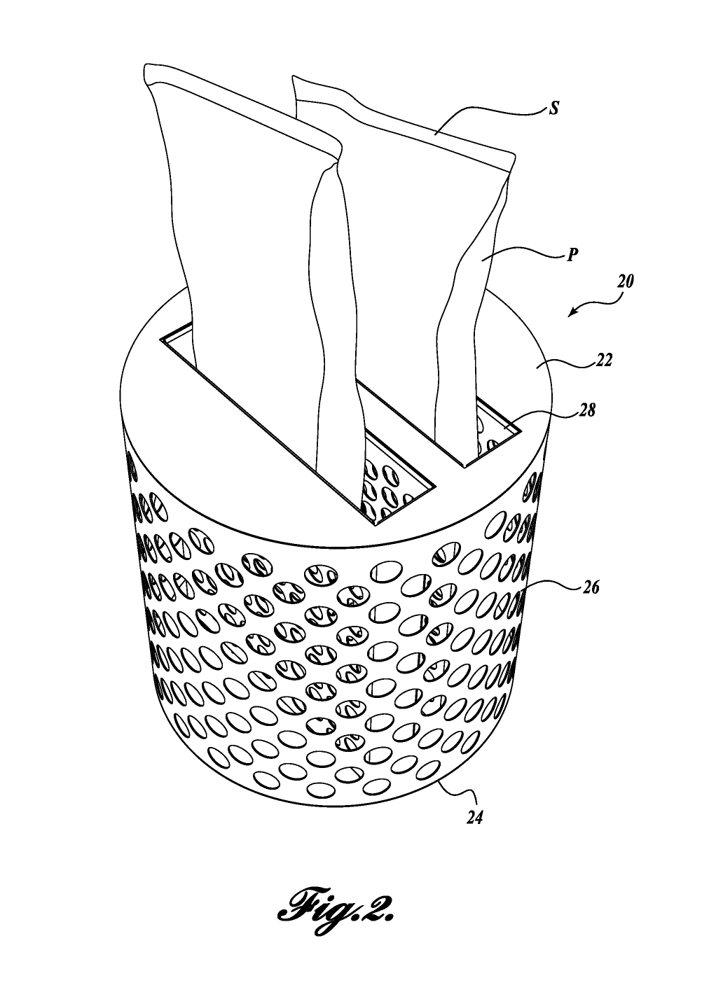

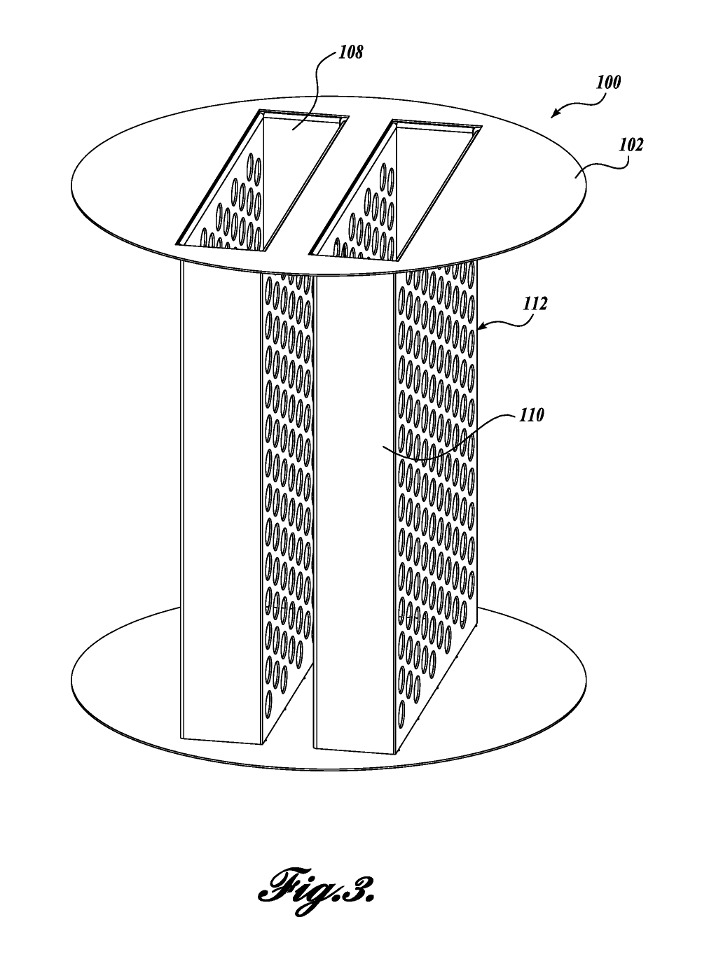

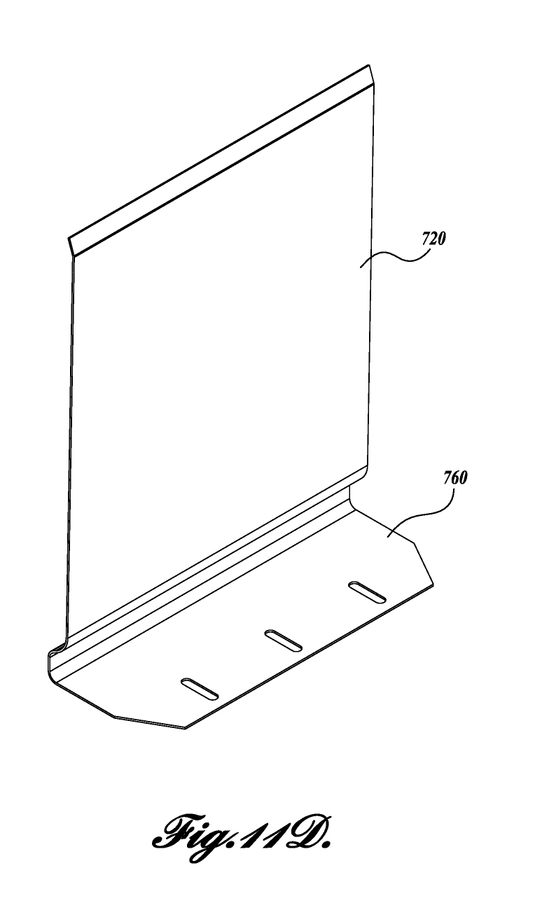

Referring to FIGS. 1 and 2, an exemplary pouch carrier 20 in accordance with embodiments of the present disclosure is shown. In the illustrated embodiment of FIG. 1, the pouch carrier 20 is designed to behave like a cylindrical can when used in a rotary sterilizer R (see also FIGS. 18-20 for other exemplary configurations and designs for rotary systems, such as rotary sterilizers). As can be seen in FIG. 2, the pouch carrier 20 has first and second circular ends 22 and 24 and a cylindrical outer shell portion 26. The first end 22 includes a plurality of sleeve openings 28 for receiving pouches P (see FIG. 3).

The pouch carrier 20 is preferably a one-piece assembly for easily receiving a pouch P without requiring assembly or disassembly. For example, the pouch carrier 20 does not have a clam shell structure, a tray assembly, or a capsule assembly, for enclosing a container on all sides after the container has been received by the carrier, as described in respective U.S. Pat. Nos. 5,245,916, 4,385,035, and 3,314,560 for previously developed technologies, the disclosures of which are hereby expressly incorporated by reference herein.

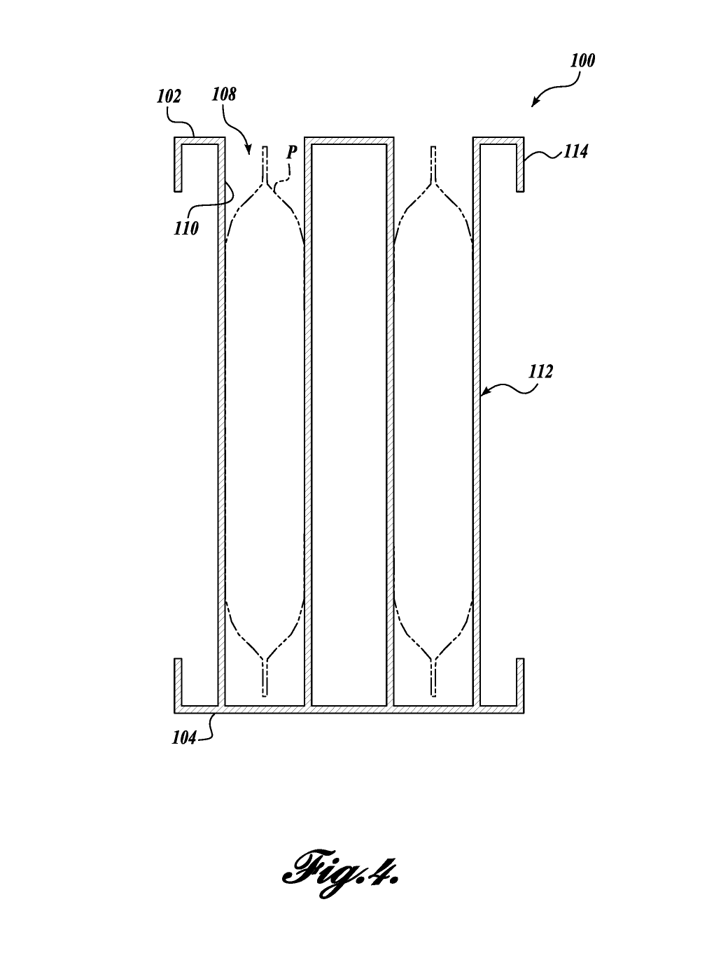

Referring now to FIGS. 3-5, an alternate embodiment of a pouch carrier 100 is shown. This embodiment is substantially similar to the pouch carrier 20 shown in FIGS. 2 and 3, except for differences regarding the outer shell. In that regard, in the illustrated embodiment of FIGS. 3-5, the carrier 100 has no outer shell, exposing the body portion 112 of the carrier, which defines the structure of the pouch sleeves 110. Like the pouch carrier 20 shown in FIG. 2, the pouch carrier 100 shown in FIGS. 3-5 is also suitably configured as a one-piece assembly. Although the pouch carrier 100 of FIGS. 3-5 does not include an outer shell, rolling action of the carrier 100 is enabled by the first and second circular ends 102 and 104 (which may include an optional outer lip 114 extending from the outer perimeter of the first and second circular ends 102 and 104, see FIGS. 4 and 5).

As seen in the illustrated embodiments, the carriers may include perforated portions. In that regard, the walls of the outer shell portion 26 in FIG. 2 and body portion 112 in FIG. 3 are shown as being perforated. Such perforations allow for efficient and consistent heat transfer to or from the pouch P during processing, for example, steam may travel through the walls of the carrier 100 to reach the pouch P. In accordance with embodiments of the present disclosure, suitable perforations may be about 0.20 inches in diameter. However, other sizes are also within the scope of the present disclosure. It should further be appreciated that, in accordance with embodiments of the present disclosure, the body portion 112 need not be made from a perforated material.

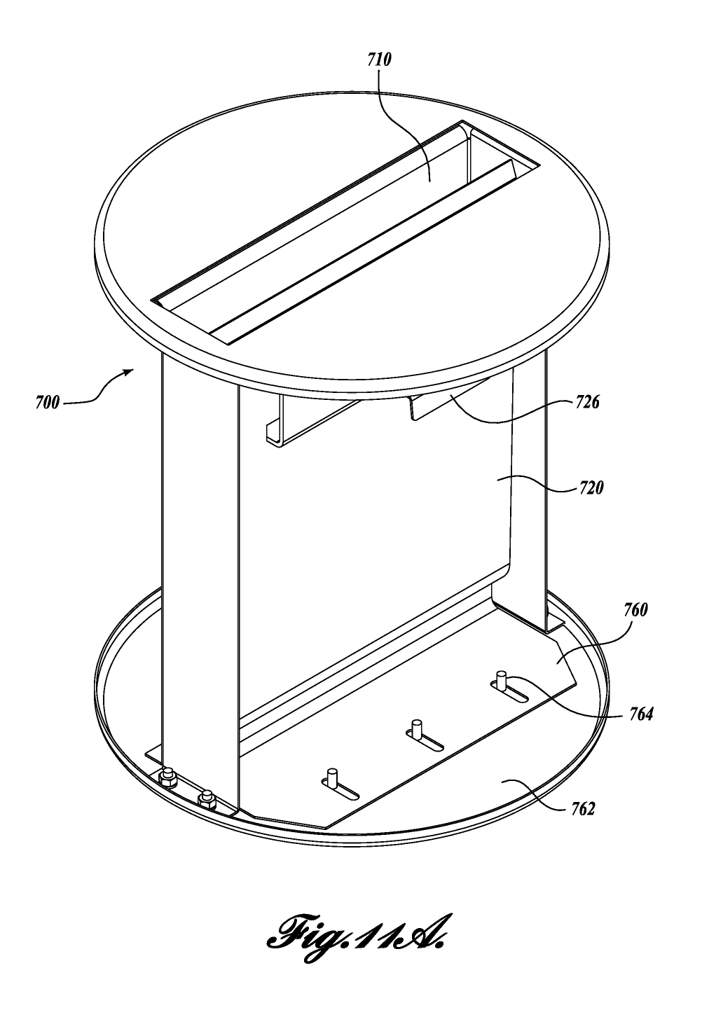

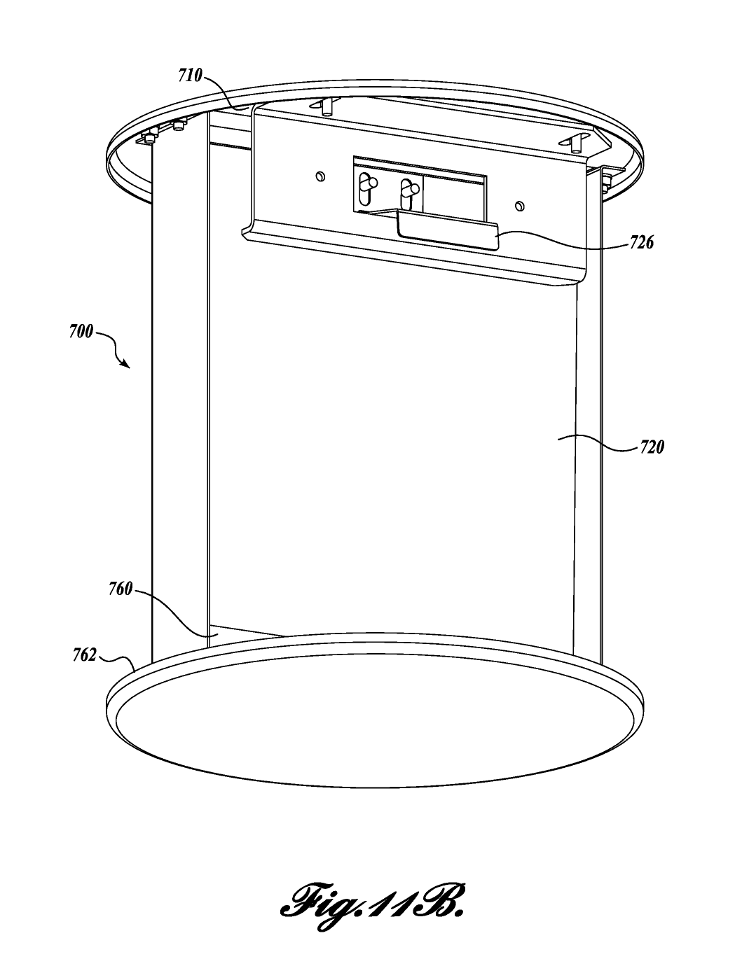

As shown in the illustrated embodiments of FIGS. 1-5, the pouch carriers 20 and 100 include two pouch sleeves. However, it should be appreciated that the pouch carriers 20 and 100 may include any number of pouch sleeves to accommodate various shapes and sizes of pouches. Further, although shown in the illustrated embodiments as having rectangular shaped cross-sections, the pouch sleeves may have other suitable cross-sectional shapes. For example, suitable cross-sectional shapes may include, but are not limited to, trapezoidal, having tapered ends, and curved, as described in greater detail below with reference to FIGS. 16A-16C.

Returning to FIG. 2, the pouches P to be carried by the pouch carriers are generally made of film laminated from layers of plastic and/or foil. The pouches P may be designed for containing various food products, including liquid products or more solid products, such as soups or stews. The pouches P generally each have a thickness when filled. Seams S around the outer peripheral edge of the pouch P contain the products. As seen in the illustrated embodiment, the pouches may be rectangular shaped or another suitable shape as deemed to be desirable for specific food products, processing parameters, or handling requirements.

Referring to the cross-sectional view of the body portion 112 in FIGS. 4 and 5, the sleeve openings 108 and the sleeves 110 are sized to allow for gentle and quick loading of the pouches P without damage to the pouches P. In that regard, the sleeve openings 108 and the sleeves 110 themselves are suitably sized and shaped to receive pouches P. The sleeves 110 may be designed to adjust the contents of the pouches P to provide for substantially even pouch thickness along the length of the pouch. In that regard, the dimensions of the sleeves 110 may provide enough resistance and gentle “squeeze” on the pouch contents to effectively retain the pouches P within the sleeves 110, even during movement of the pouch container 100 (see, e.g., movement in the rotary sterilizer shown in FIG. 1). Referring to FIG. 4, the pouch carrier 100 is shown holding two larger sized pouches P. Referring to FIG. 5, the pouch carrier 100 is shown holding four smaller sized pouches P in a stack of two in each sleeve 110.

After pouches P have been received in the sleeves 110, the sleeves 110 may include a retention system to further retain the pouches P within the sleeves 110 and/or to more evenly distribute the pouch contents within the pouch P for more even pouch thickness distribution within the sleeve. Suitable retention systems are used to minimize pouch movement when the pouch carrier 100 moves to minimize scuffing or wrinkling of the pouches P. Minimizing pouch scuffing and wrinkling not only preserves pouch aesthetics, but also pouch integrity. Moreover, retention systems that provide more even pouch thickness distribution within the sleeve 110 will provide for more consistent heat transfer to the contents in the pouches P. As a one-piece assembly, suitable retention systems are designed so that they do not separate from the body portion 112 of the carrier 100.

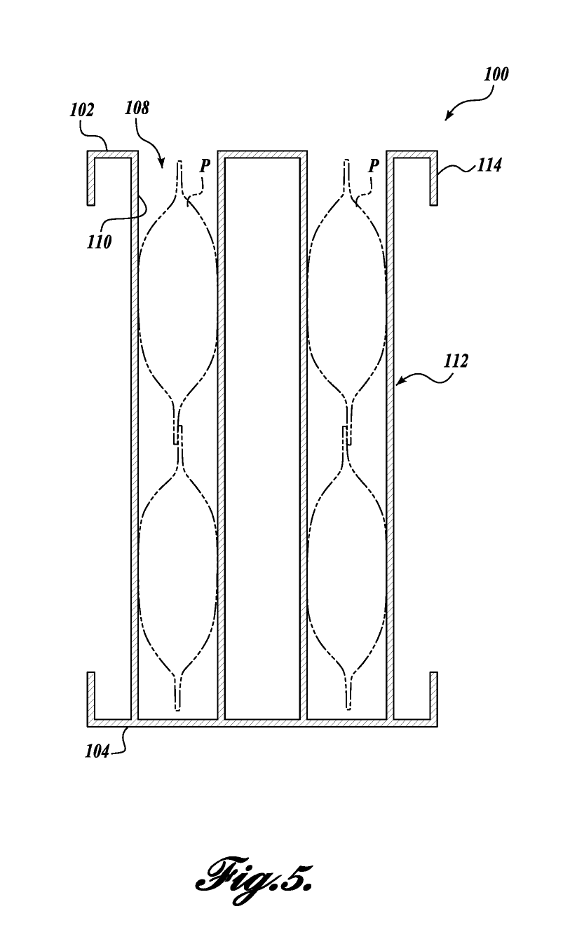

With reference to FIGS. 6A-17, various embodiments of pouch loading and retention devices are shown. For example, referring to FIG. 6A, the outer sleeve wall 220 may be movable from its normally biased closed position (see right side sleeve for normal biased position) to open the sleeve 210 beyond its normal size to allow a user to easily slide a pouch P into the sleeve 210 (see left side sleeve for opened position). When the outer sleeve wall 220 is released, it biases back toward its normal position to gently “squeeze” the pouch P.

In the illustrated embodiment of FIG. 6A, the outer pouch wall 220 is coupled by a hinge 222 to the second end 204 of the body portion 212. A biasing member 224 (such as a compression coil spring or other type of biasing member) adjacent the first end 202 of the body portion 212 is normally biased in the closed position (such as the parallel wall position shown in FIG. 6A). When loading and unloading, a pulling device D, such as a stationary cam or a pull actuator, can be used to engage with a handle 226 to pull the outer wall 220 to the opened position. After the pouch P has been loaded, the bulge of the pouch P may resist the outer wall 220 as it extends to the closed position, thereby holding the pouch in a gently “squeezed” position.

Of note, the opening 208 in the first end 202 of the body portion 212 is larger than the normally biased closed position to allow for loading of the pouch when the outer pouch wall 220 is in the opened position. Referring to FIG. 6B, the opening 208 in the first end 202 of the body portion 212 may include an optional lip 214 extending inwardly from the outer wall 220 to further assist with retention of the pouch P in the sleeve 210.

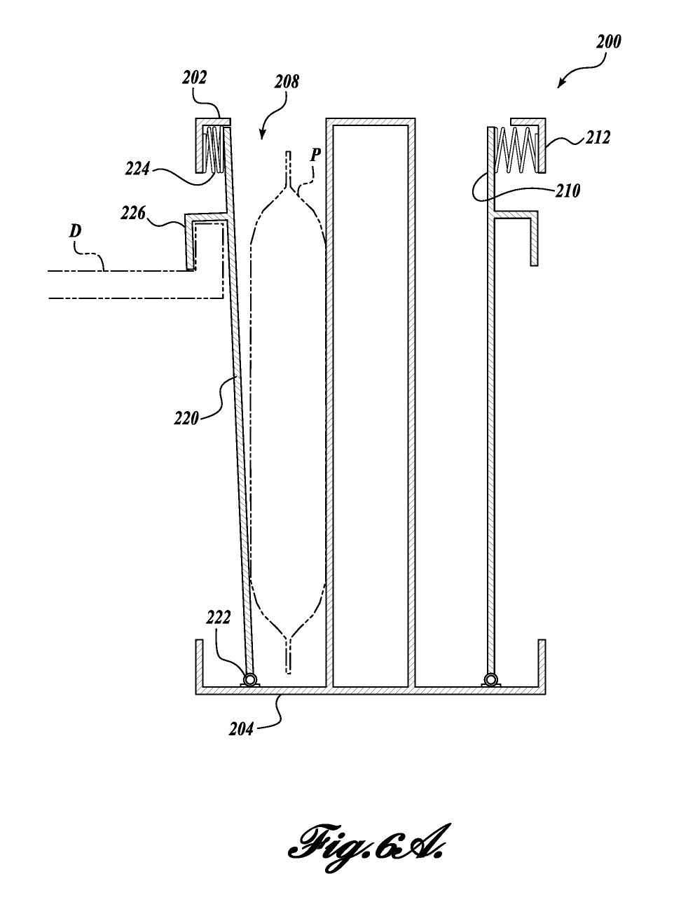

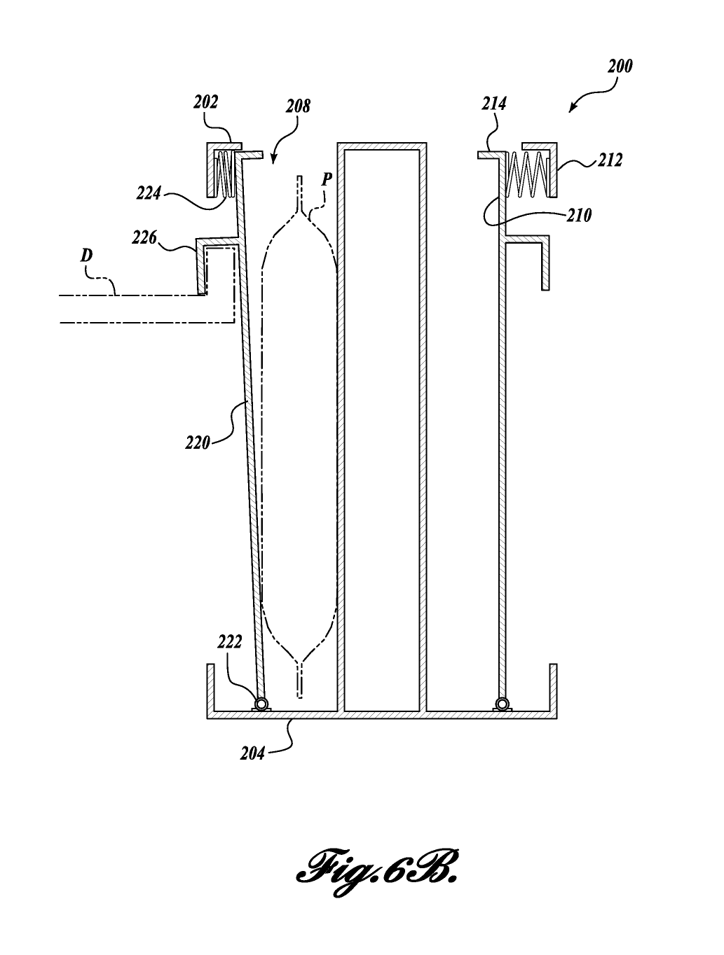

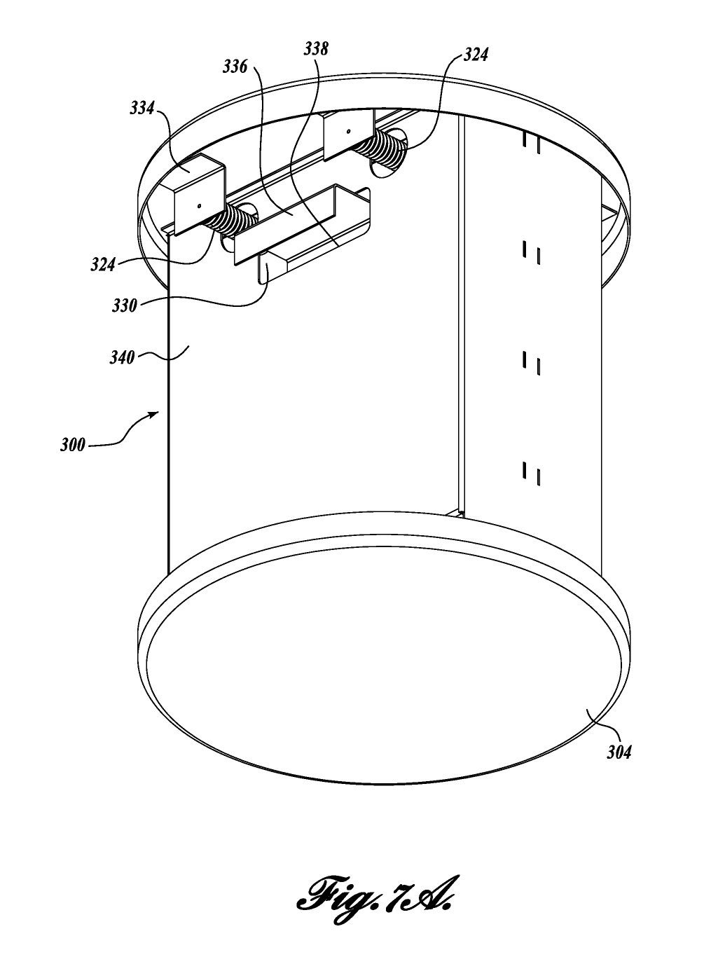

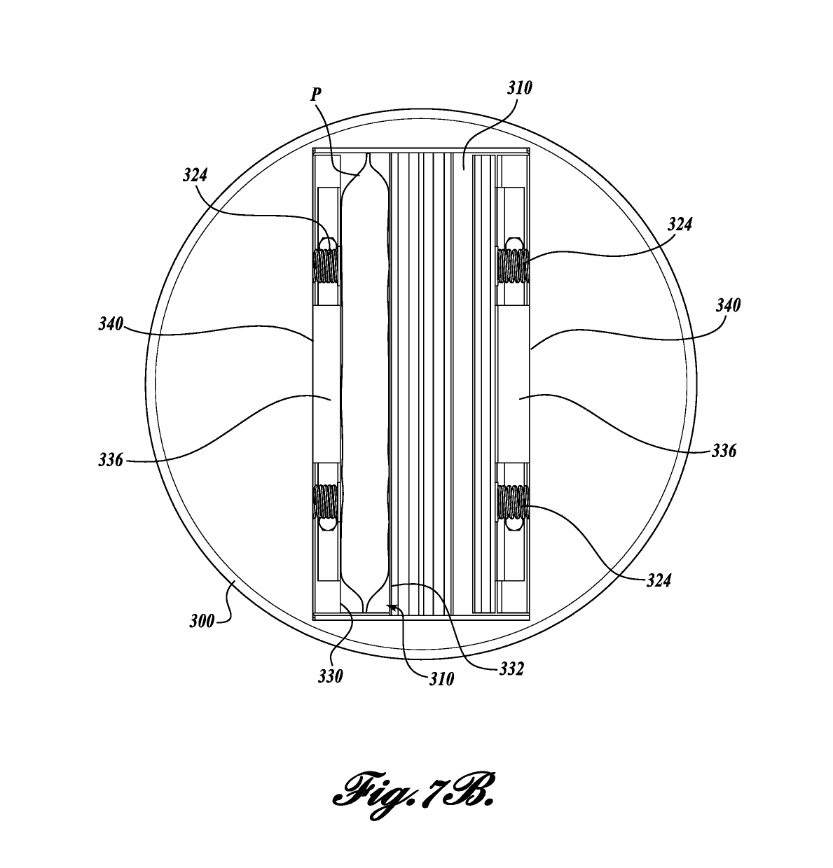

Referring to FIGS. 7A and 7B, respective side isometric and top views of an exemplary hinged retention carrier 300 (similar to the embodiments in FIGS. 6A and 6B) are shown. The sleeves 310 (see FIG. 7B) includes first and second upright sleeve walls 330 and 332. The first sleeve wall 330 is rotatable relative to the second end 304 of the carrier 300 about a hinge (not shown, but positioned along the bottom of wall 330). Biasing members 324 (shown as coil springs) are attached to stationary brackets 334. The biasing members 324 bias the first sleeve wall 330 to a closed position (see FIG. 7B). Handle 336 (see FIG. 7A) can be grasped or otherwise used to pull the rotatable first sleeve wall 330 to an open position to insert or release the pouch P (shown in the left side FIG. 7B to be contained within the sleeve 310). In the illustrated embodiment, handle 336 is pulled through a hole in a stationary wall 340.

The carrier 300 may further include an adjustable stop (not shown) to limit the travel distance of the first sleeve wall 330 to the closed position. As a non-limiting example, the adjustable stop may be a bolt or threaded stud attached to the first sleeve wall 330 and passing through a hole 338 in the stationary wall 340. The adjustable stop may, for example, includes adjustable nuts which rest against the stationary wall 340 or at least one of the stationary brackets 334 to limit the travel distance of the second sleeve relative to the stationary wall 340 or at least one of the stationary brackets 334.

Referring now to FIG. 8, another embodiment of a hinged retention carrier 400 is shown (similar to the embodiments of FIGS. 6A-7B). The carrier 400 includes a position limiter assembly 440 to limit the travel distance of the second sleeve wall 420 coupled to hinge 422. The position limiter assembly 440 includes a position limiting device 442 that extends outwardly from the outer sleeve wall 420, and a stationary surface 412 to which the position limiting device 442 may releasably couple.

In the illustrated embodiment of FIG. 8, the position limiting device 442 includes a first contact surface 446 and a second contact surface 448, which are capable of coupling with the stationary surface in two different sleeve positions, for example, a processing position and a loading/unloading position (e.g., compare positioning of right side sleeve 410 with left side sleeve 410 in FIG. 8). In the illustrated embodiment, the stationary surface 412 includes a substantially vertical wall 450, for example, projecting downward from the first end 402 of the carrier 400. The stationary surface 412 further includes a slot or hole 452 through which the position limiting device 442 is received.

It should be appreciated that the position limiting device 442 may be made from a flat spring material, such that it is capable of bending or flexing when in either of the first and second sleeve positions (e.g., compare positioning of right side sleeve 410 with left side sleeve 410 in FIG. 8). In that regard, looking at the right side sleeve 410, the first contact surface 446 of the position limiting device 442 passes through slot or hole 452 and engages with an upper outer surface of the substantially vertical wall 450 of the stationary surface 412. This engagement maintains the sleeve 410 in a position for pouch retention to a width Wt and prevents the outer sleeve wall 420 from collapsing inwardly.

Now looking to the left side sleeve 410, the second contact surface 448 of the position limiting device 442 engages with a lower outer surface of the substantially vertical wall 450 of the stationary surface 412. The transition from the first position (see right side sleeve 410) to the second position (see left side sleeve 410) is achieved by pulling the outer sleeve wall 420 outwardly (for example, using pulling device D to hook downwardly extending flange 426) from the first position to the second position. As the position limiting device 442 moves outwardly through hole 452, the second contact surface 448 engages with the lower outer surface of the substantially vertical wall 450 of the stationary surface 412. In the second position, the sleeve 410 will remain open for loading or unloading.

The second contact surface 448 in the illustrated embodiment, is shown as a catch or tab with a cam ramp surface that is configured to engage with a lower edge of slot 452. The cam ramp surface allows for the transition from the first position to the second position while minimizing the risk of the position limiting device 442 catching en route. To transition from the second position back to the first position, the position limiting device 442 is lifted upwards to disengage the second contact surface 448 from the lower outer surface of the substantially vertical wall 450 of the stationary surface 412.

In the illustrated embodiment, the second sleeve wall 420 is normally biased in the closed position, with the wall being biased to a tapered position, having a smaller nominal sleeve width Wt at the top of the sleeve than at the bottom of the sleeve Wb. The inventors have found that a sleeve having a tapering width (as shown narrower at the top Wt and wider at the bottom Wb) helps to maintain the pouch (not shown) in the sleeve. It should further be appreciated that the sleeve closed position can be designed and dimensioned (depending on the size and shape of the specific pouch to be received) to substantially evenly distribute the contents in the pouch by bearing against a substantial surface area of the pouch. Such substantially even distribution can provide for more even heat transfer to the contents in the pouch for predictable and repeatable thermal processing.

Referring now to FIG. 9, another embodiment of a pouch retention device is shown. Like the embodiments shown in FIGS. 6A-8, the outer sleeve wall 520 in the illustrated embodiment of FIG. 9 may also be movable from its normally biased closed position (see right side sleeve for normally biased closed position) to open the sleeve 510 beyond its normal size to allow a user to easily slide a pouch P into the sleeve 510 (see left side sleeve for opened position). When the outer sleeve wall 520 is released, it biases back toward its normal position to gently “squeeze” the pouch P. However, the outer wall 520 in the illustrated embodiment of FIG. 9 is not hinged like the outer walls 220, 320, and 420 in the illustrated embodiments of FIG. 6A-8. In that regard, outer wall 520 in FIG. 9 acts like a leaf spring and is bendable from its normal closed position to its opened position.

Now referring to FIG. 10A, a position limiter assembly 640 for a leaf spring-type carrier 600 is shown. In the illustrated embodiment, a carrier 600 is shown having two sleeves 610, one on the left side in a normal open loading/unloading position, and one on the right side in a closed processing position. Referring first to the open sleeve 610 on the left side of the drawing, the second sleeve wall 620 is biased in the open position and the position limiter assembly 640 extends unengaged through a slot or hole 652 in the substantially vertical wall 650 of the stationary surface 612. In the embodiments of FIG. 8, the substantially vertical wall 450 of the stationary surface 412 extends for only a portion of the distance between the top and bottom ends of the carrier 400. However, in the illustrated embodiment of FIG. 10A, the substantially vertical wall 650 of the stationary surface 612 extends for the entire distance between the top and bottom ends of the carrier 600.

Referring to FIGS. 10A and 10B, a stopping device 648 (shown as a finger) may extend from the bottom surface of the position limiter assembly 640 to prevent inadvertent opening and control the closing width of the sleeve 610.

To close the open sleeve 610 on the left side of the drawing, the position limiter assembly 640 is pushed to the right until the contact surface 648 (shown as a pierced tab) disengages the lower inner surface of the substantially vertical wall 650 of the stationary surface 612 (for example, similar to the position shown on the rights side of the drawing, but in mirror image). Referring now to the closed sleeve 610 on the right side of the drawing, the contact surface 648 engages the lower inner surface of the substantially vertical wall 650 of the stationary surface 612, and the sleeve 610 is maintained in a closed position. To open the closed sleeve 610 on the right side of the drawing, the position limiter assembly 640 is pushed up to release the engagement of the contact surface 648 and the lower inner surface of the substantially vertical wall 650 of the stationary surface 612. The second sleeve wall 620 then moves outwardly toward the substantially vertical wall 650 to rest in an open position (for example, similar to the position shown on the left side of the drawing, but in mirror image).

It should be appreciated that the carriers described herein are designed with robust position limiting devices, such that the carriers will remain in either of their designated open or closed positions as they travel through handling and processing shocks. Moreover, it should be appreciated that the carriers may be designed to have one or more position limiting assemblies that are capable of stopping in more than two (e.g., open and closed) positions. For example, the carriers may have different open positions designed for different sized pouches. Although the embodiments described herein generally include biasing members (such as compression springs or leaf springs), it should be appreciated that carriers without biasing members are also within the scope of the present disclosure.

Referring now to FIGS. 11A-11D, views of an exemplary leaf spring-type carrier 700 (similar to the embodiment in FIGS. 10A and 10B) are shown. Although similar numbers are used for like parts in FIGS. 10A and 10B, it should be noted that the carrier 600 shown in FIGS. 10A and 10B is a double sleeve carrier, and the carrier 700 shown in FIGS. 11A-11C is configured as a single sleeve carrier.

The sleeve 710 for receiving a pouch (not shown) is seen in FIGS. 11A and 11B. As can be seen in FIGS. 11A and 11B, the second sleeve upright wall 720 is biased to a closed position, which can be opened by a user grasping or otherwise using handle 726.

Referring to FIG. 11D, the second sleeve wall 720 (shown as a leaf spring-type wall) is removable from the carrier 700. This type of removable wall allows for modification to the carrier 700, for example, to exchange parts (such as sleeve walls) to accommodate other types of pouches having varying size or shape, or for maintenance or cleaning. In the embodiment shown, the removable spring wall 720 includes a base 760 that is attachable to an end 762 or other portion of the carrier 700 by coupling devices 764 (see FIG. 11A). In addition, the removable spring wall 720 of the embodiment shown is designed to have a biasing action into the closed position to hold and substantially evenly distribute the contents of a pouch having a specific size and shape within the sleeve 710. It should be appreciated, however, that other biasing designs are also within the scope of the present disclosure, for example, biasing to the open position.

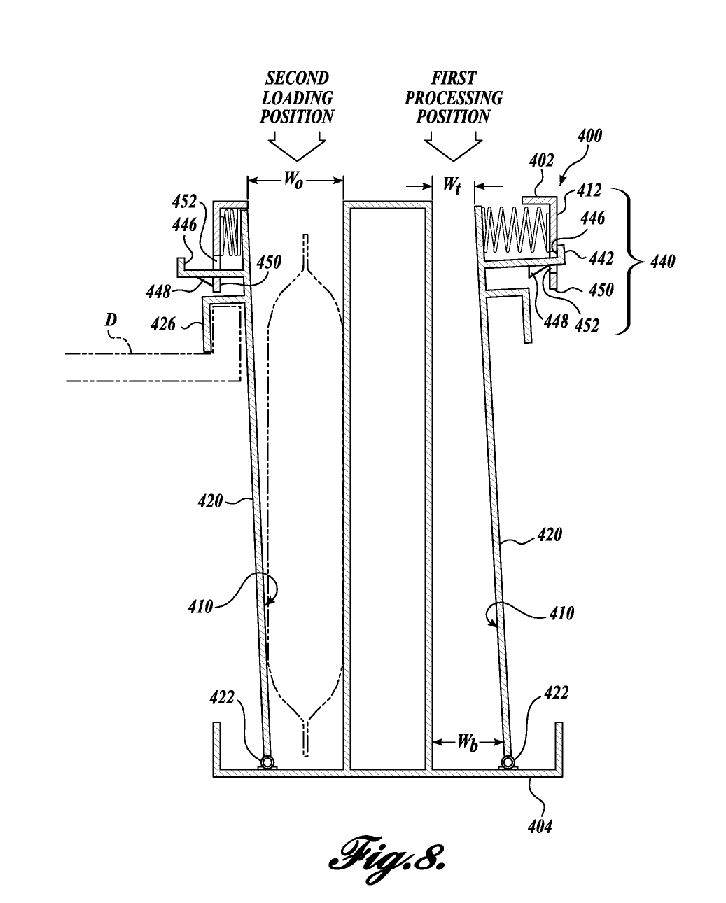

Various other embodiments of pouch retaining and release systems will now be described. Referring to FIGS. 12A-13B, instead of a wall being movable (as can be seen in the illustrated embodiments of FIGS. 6A-11D), fingers 830 and 930 extend from or through respective outer walls 820 and 920 are used to “squeeze” the pouches P. Referring to FIGS. 12A and 12B, the fingers 830 extend from the wall and are bent at an angle to apply compression force on the pouch P. Like the outer wall 520 shown in the illustrated embodiment of FIG. 9, the fingers 830 in the illustrated embodiment in FIGS. 12A and 12B are not hinged to the outer wall 820. Rather, they act as leaf springs, being bendable from a normal compressing position to an opened position. In that regard, the fingers may be laser cut into the outer wall, then formed with a bend configuration to provide suitable “squeeze” against the pouch P. The fingers 830 may include a handle or another pulling device (not shown) to pull them from the normal compressing position to the opened position.

In the illustrated embodiment of FIGS. 13A and 13B, the fingers 930 are attached to a hinge and torsion spring assembly 932 to rotate the fingers 930 against the pouch P. Like the illustrated embodiment of FIGS. 12A and 12B, fingers 930 may include a handle or another pulling device (not shown) to pull them from the normal compressing position to the opened position.

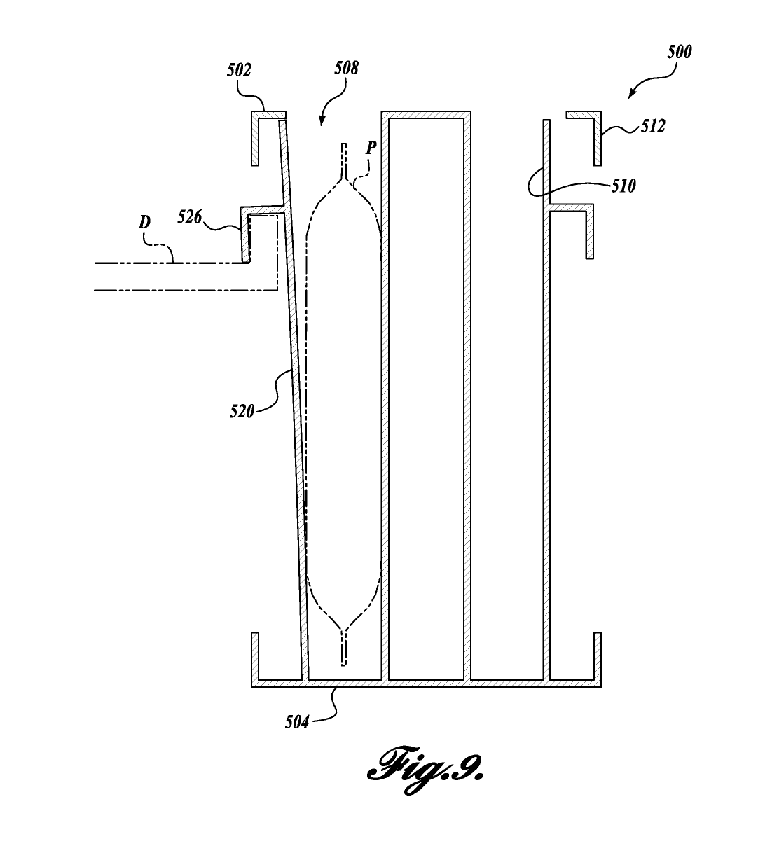

Referring to FIGS. 14A and 14B, a body portion 1012 having movable inner walls 1040 and stationary outer walls 1020 in the sleeves 1010 is shown. In that regard, the inner walls 1040 are coupled by hinges 1042 to the second end 1004 of the body portion 1012. A biasing linkage system 1044 can be used to move the inner side walls 1040 between a normal compressing position (see FIG. 14A), and an opened position (see FIG. 14B).

The linkage system 1044 generally includes a cross bar 1046 biased by a biasing member 1024 (such as a compression coil spring) into the normal compressing position (see FIG. 14A), but movable to the opened position (see FIG. 14B). The cross bar 1046 is pivotally coupled to links 1048 that are pivotably coupled to the inner walls 1040. As the cross bar 1046 is pushed down, the links 1048 pivot and push the inner walls 1040 inward. The inner walls 1040 pivot around hinges 1042 to enlarge the openings 1008 of the sleeves 1010. A pushing device (not shown) may be used to generate a force (shown as arrow F) on the cross bar 1046 to activate the biasing linkage system 1044.

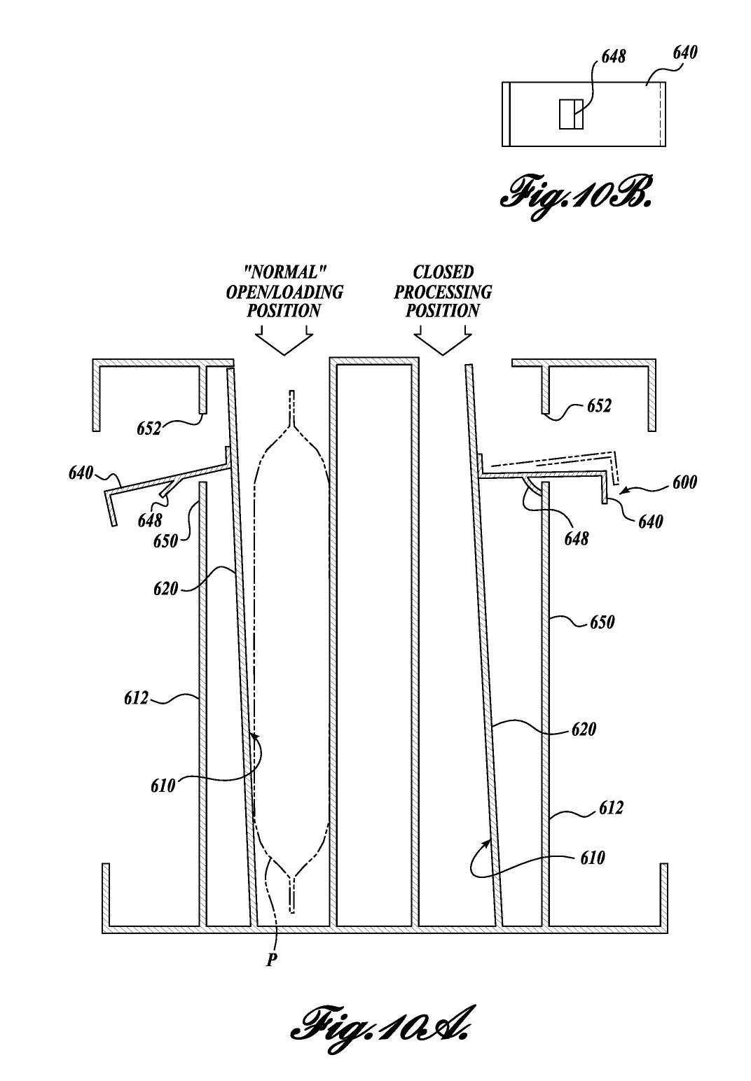

Referring now to FIGS. 15A and 15B, another embodiment of a pouch carrier 1100 having a pouch loading and retention system is shown. In the illustrated embodiment of FIGS. 15A and 15B, the substantially cylindrical pouch carrier 1100 includes first and second portions 1150 and 1152 defining first and second pockets 1154 and 1156. The first and second portions 1150 and 1152 are divided along a plane extending longitudinally through the diameter of the first and second circular ends 1102 and 1104. The first and second portions 1150 and 1152 are hingedly coupled to one another by a hinge 1160 and each includes a door 1162 and 1164 hingedly coupled so as to close each of the respective first and second pockets 1154 and 1156. A carrier latch 1166 secures the carrier 1100 in the closed position.

During pouch loading, the pouch carrier 1100 is opened to receive first and second pouches P. The pockets 1154 and 1156 are shaped to constrain pouch motion, and in that regard, may be specifically designed for certain kinds of pouches or generally designed to constrain the motion of most pouches. After the pouches P have been loaded, the doors 1162 and 1164 are closed to snugly fit over the pouches P and constrain pouch motion. After the pouches have been loaded, at least one of the first and second portions 1150 and 1152 is rotated around hinge 1160 to latch together with latch 1166 and form a cylinder.

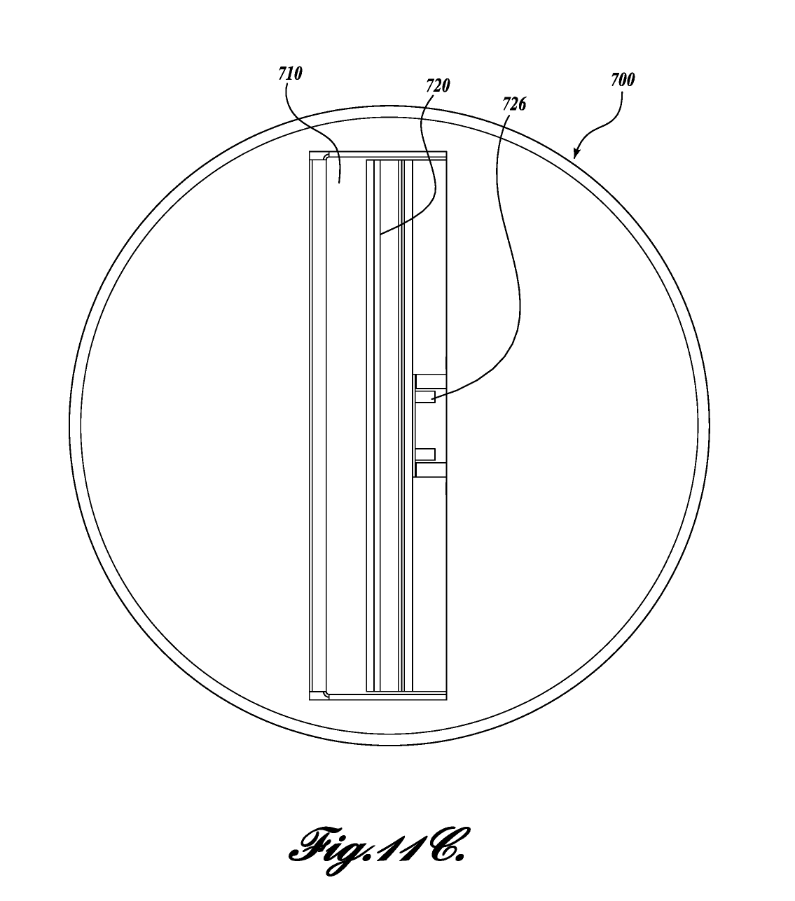

In addition to sleeve retention systems, as described above, sleeve cross-sectional shape may also assist with pouch retention to minimize movement of the pouch P within the sleeve and to more evenly distribute the contents of the pouch P throughout the entire pouch P. Referring to FIGS. 16A, 16B, and 16C, suitable cross-sectional shapes may include, but are not limited to, trapezoidal (see FIG. 16A), with tapered ends (see FIG. 16B) to accommodate the seal S along the outer peripheral edge of the pouch P (see FIG. 2), and curved (see FIG. 16C). Referring to FIG. 17, a trapezoidal sleeve shape (for example, as shown in FIG. 16A) may be combined with a hinged sleeve retention mechanism, such as that shown in FIGS. 14A and 14B.

Now referring to FIGS. 18-20 various designs for rotary systems, such as rotary sterilizers, are shown. It should be appreciated that rotary systems may be designed to handle any number of carriers at any given time. In FIG. 1, a test rotary sterilizer is shown, which is capable of handling eight carriers at any given time. Suitable larger diameter rotary sterilizers for use in production may be capable of processing thousands of carriers at any given time.

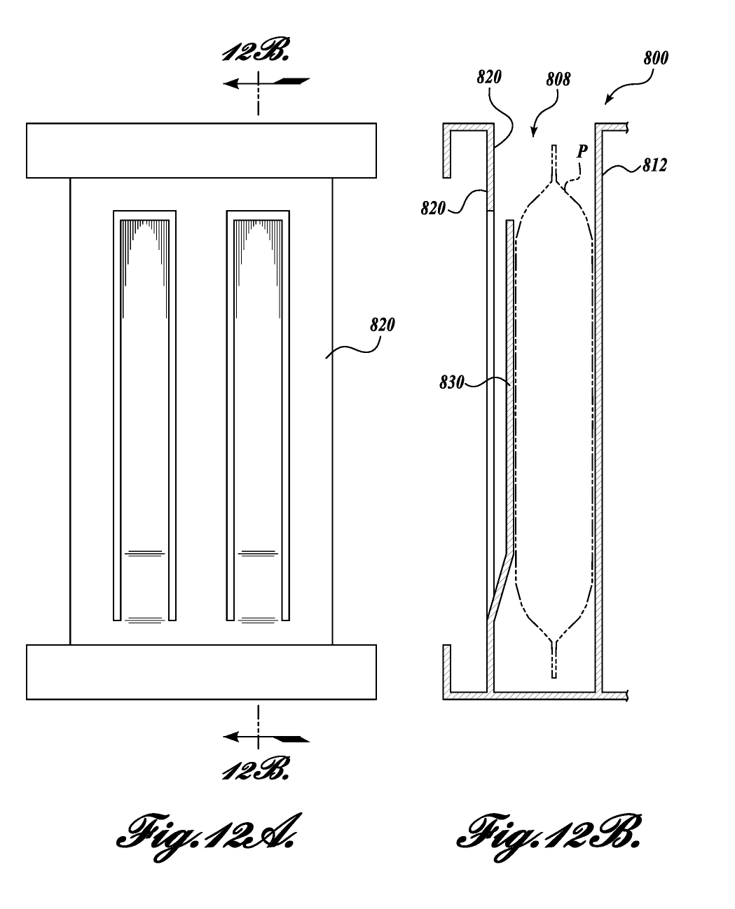

It should be appreciated that the rotary systems may have an agitating reel (see FIG. 19) or a non-agitating reel (see FIG. 20). Referring to FIGS. 18 and 19, and as described above, rotary sterilizer 1200 includes an agitating reel 1202. A rotary valve 1204 feeds the reel 1202 of the rotary sterilizer 1200 with carriers 1206. The reel 1202 is surrounded by an outer shell 1208. The carriers 1206 are rotated around the reel 1202 and are maintained in position along the reel 1202 by angles 1210 (see FIG. 19). In certain positions along the reel 1202, the carriers 1206 may be supported by the outer shell 1208. In that regard, when the carriers 1206 are on the upper portion of the reel 1202 they are supported by the reel 1202 itself, but when they are on the bottom portion of the reel 1202, they are supported by the outer shell 1208 and pushed along the outer shell 1208 by the angle 1210 located behind the carrier 1206 (see FIG. 19). When supported by the outer shell 1208, the carriers 1206 roll along the internal surface of the outer shell 1208 and therefore the carrier contents are agitated by such rolling action.

Referring now to FIG. 20, a portion of rotary system 1300 including a non-agitating reel 1302 is shown. The rotary sterilizer of the illustrated embodiment of FIG. 20 is substantially similar to the illustrated embodiment of FIGS. 18 and 19, except for a difference regarding the angles 1310. In that regard, the angles 1310 are designed to carry and support the carriers 1306 when carriers 1306 are on the bottom portion of the reel 1302. Because the carriers 1306 do not roll along the internal surface of the outer shell 1308, the carrier contents are not agitated by any rolling action.

With canned food, agitation may be desirable for certain food types, for example, milk and other low-viscosity foods. With other fragile foods, for example, lima beans, agitation may not be desired because it may cause the beans to become broken or damaged. The advantage of using a non-agitating rotary sterilizer with the many embodiments of pouch carriers described herein is that agitation is minimized, which in turn minimizes scuffing and wrinkling of the carried pouches P. Therefore, in accordance with methods of the present disclosure for processing pouches in rotary sterilizers, pouches may be placed in cylindrical carriers and received in either agitating or non-agitating rotary systems, such as rotary sterilizers.

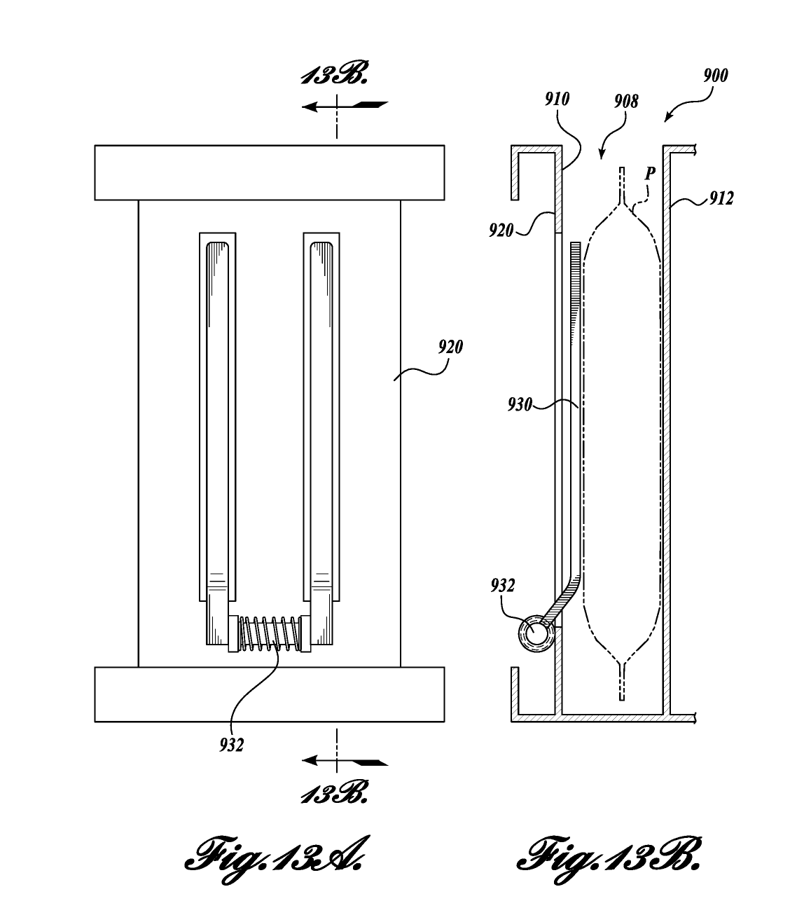

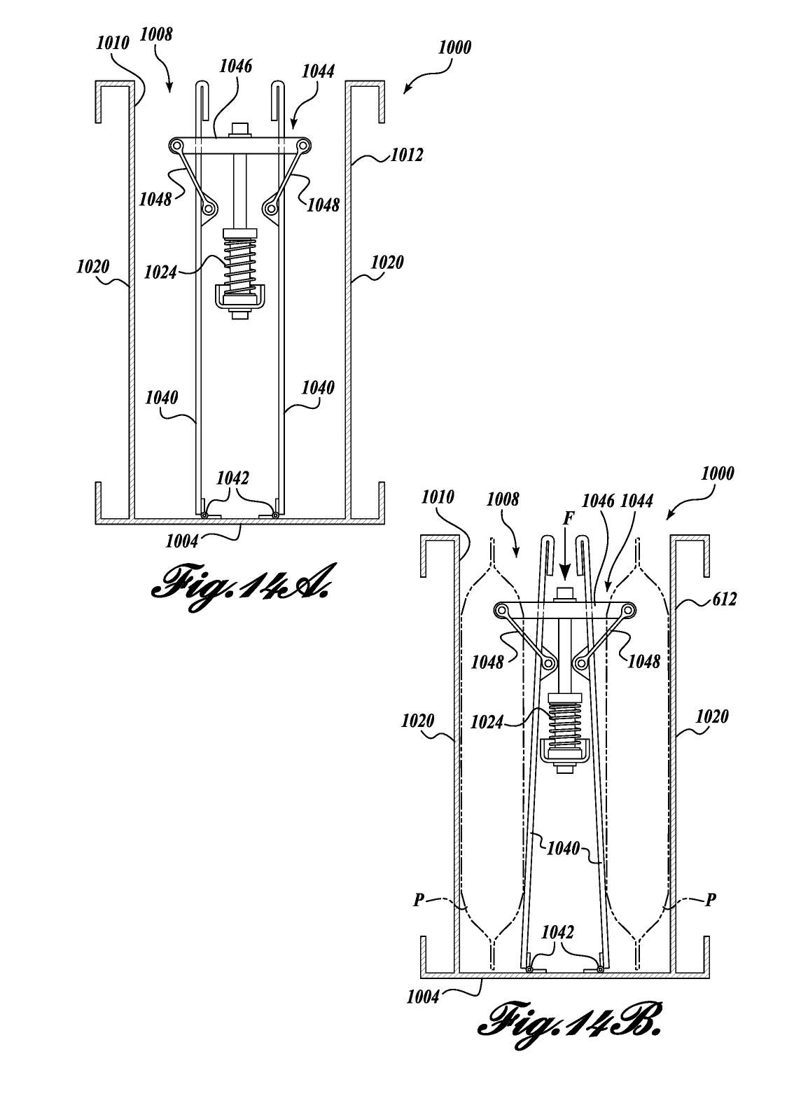

Referring to FIGS. 21-23, intermittent and continuous unloading systems 1340 and 1350, as well as a chute 1356 for receiving pouches that are dropped downwardly from a carrier, are shown. Referring to FIGS. 21 and 22, the carriers 600 are supported by two laterally spaced apart conveyors 1352 positioned far enough apart that the pocket openings of the carriers 600 are positioned over the open width 1354 between the conveyors 1352. In the illustrated embodiments of FIGS. 21-23, the carriers do not rotate on the conveyors, but rather are supported by the conveyors and/or by guide rails positioned beneath either of the first or second ends of the carrier (see, e.g., FIG. 23 showing conveyors 1352 positioned beneath carrier 600). This support system maintains the carriers in a non-rotating position, preferably with the openings to the sleeves oriented downwardly.

In addition to conveyors 1352 for support, the systems 1340 and 1350 may also include an optional alignment feature. Using an alignment feature, the carrier must be rotated or otherwise indexed to a correct position for loading or unloading pouches. For example, using the geometry of the carrier, if the central box containing the sleeves is rectangular in shape, a carrier can thus be guided along conveyors such that the long side of the box is parallel to travel. The orientation of the pocket openings is thus known. In the illustrated embodiment of FIG. 23, guide rails 1358 run the length of the conveyors 1352, are parallel to the conveyors 1352, and are configured for maintaining the carriers 600 on the conveyors 1352. In that regard, the carriers 600 may be top heavy, and the guide rails 1358 will prevent them from toppling over.

At the appropriate time, the carriers 600 are acted upon to allow the pouches P contained in the sleeves to drop downwardly from the sleeves (largely by gravity). Referring to FIGS. 21 and 23, pulling devices D (for example, as shown and described with reference to FIGS. 8 and 9) may be activated to move a carrier wall from a pouch retaining position to a pouch release position. Referring to FIG. 22, a pulling bar B may be positioned along the path of the conveyors 1352 to move a carrier wall from a pouch retaining position to a pouch release position. Such a pulling bar may be active, as described with reference to FIGS. 21 and 23, or it may be passive, for example, using a stationary bar with sloped surfaces that “cam” the carrier wall to the pouch release position as the carrier 600 moves along the conveyors 1352.

The pouches P will travel between the two supporting conveyors 1352 downwards onto appropriate chutes 1356 or take-away conveyors (see, e.g., FIG. 23). The action of unloading and dropping pouches could occur while the carrier is either stopped (for example, at an “unload position” showing in FIG. 21), or is moving along the support conveyors (for example, see “unload zone” in FIG. 22). It should be appreciated that a continuous unloading process may allow for faster system operating conditions by reducing carrier stoppages.

Referring to FIGS. 21 and 22, mechanisms can be activated at the “unload position” 1360 or “unload zone” 1370 to actuate a latch mechanisms and open the sleeve of the container 600. To allow integration with a full processing system, such as loading, unloading, and controls, other features can be included in the carrier design, as described in greater detail below. As one non-limiting example, the carriers may include a “pouch present” feature. Such a feature may include holes in the walls of the carrier to allow photo eyes (for example) to shine through the carrier to verify if the pocket does or does not contain a pouch.

As another non-limiting example, the carriers may include “sleeves up” feature for determining if the carrier is correctly oriented for loading or unloading: sleeves upward, or downward, as required. For example, the pocket openings can be sensed using eddy current proximity sensors, magnetic sensors, or other appropriate sensors, and appropriate action can be taken to remove incorrectly oriented carriers.

It should be appreciated that the carrier unloading processes of FIGS. 21-23 may be adapted for use with any of the above-described embodiments carriers. However, the carriers illustrated in FIGS. 21-23 generally include release mechanisms for unloading pouches (such as position limiter assemblies shown in FIGS. 10A and 10B).

In accordance with these processes, carriers (as described above) can be unloaded after processing by rotating or otherwise orienting the carriers with the openings of the sleeves directed downwards, and aligning the carrier so that the long side of the sleeve is, for example, parallel to the path of travel along a conveyor. Likewise, carriers can be loaded by orienting the carriers with the sleeves upwards, aligning the carrier so that the long side of the sleeve is, for example, parallel to the path of travel along a conveyor.

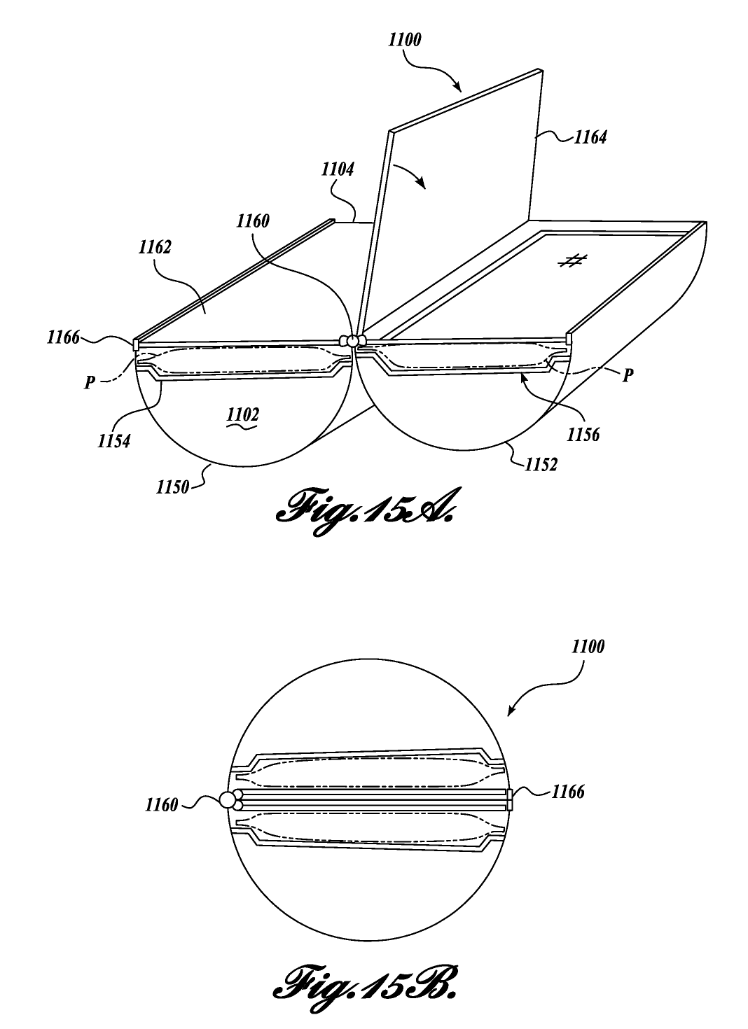

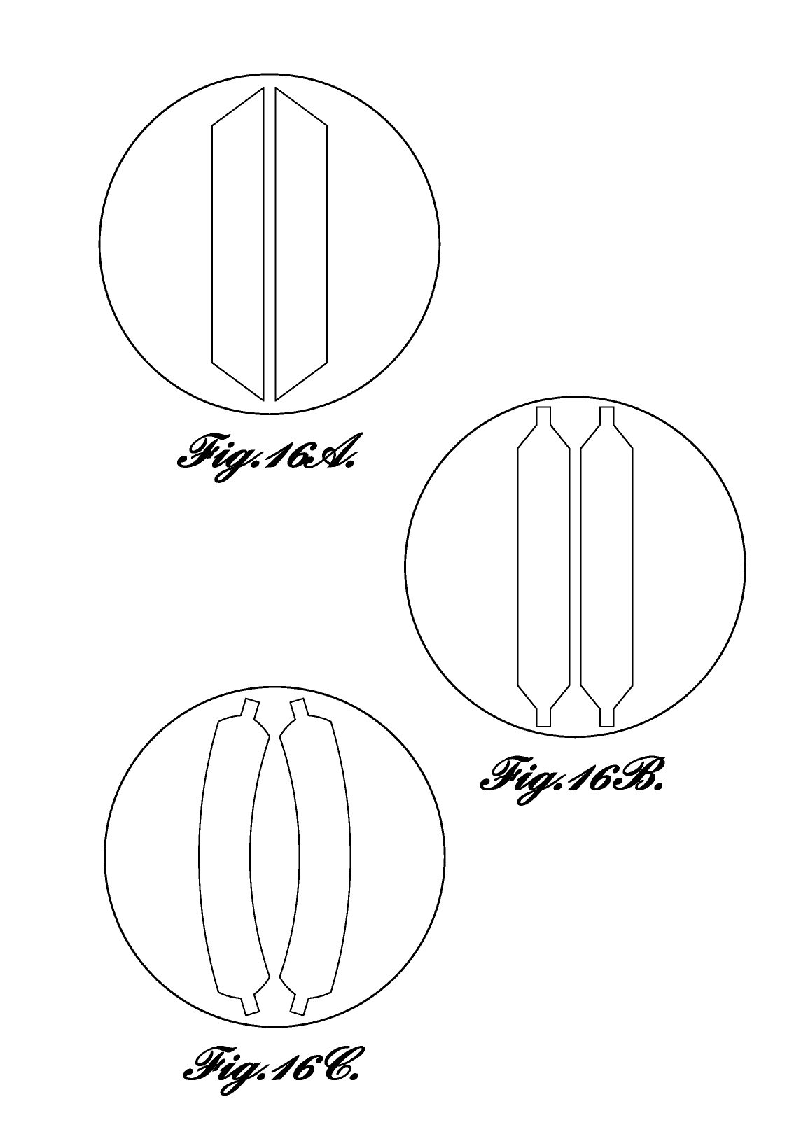

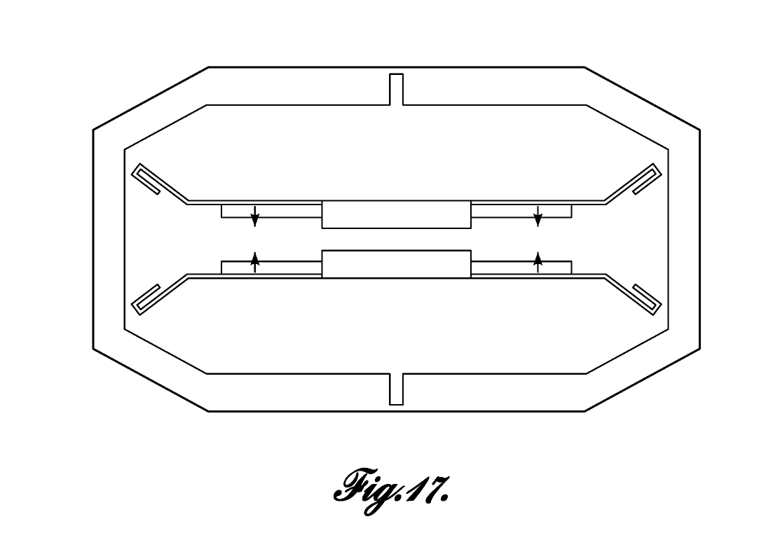

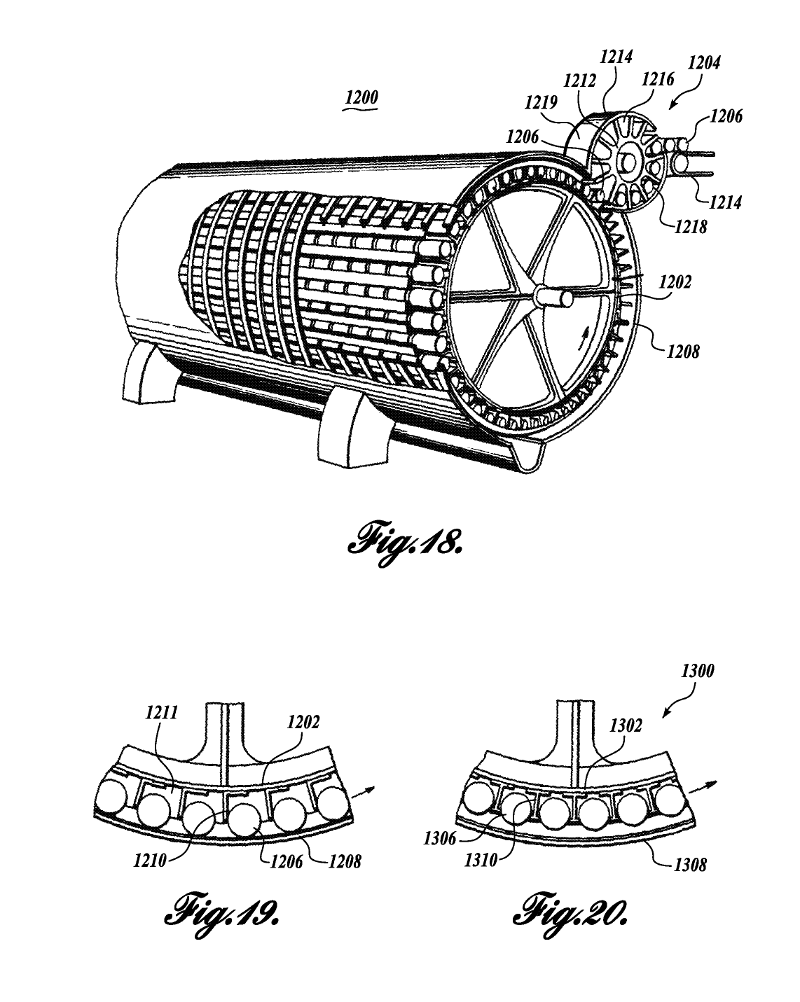

Now referring to FIGS. 24-27, other carrier embodiments are shown for carrying other types of containers besides pouches. In that regard, FIG. 24 is a bottle carrier 1400, FIG. 25 is a tub carrier 1500, and FIG. 26 is a fiberboard and plastic laminate container carrier 1600, all in accordance with embodiments of the present disclosure. In accordance with embodiments of the present disclosure, the carriers 1400, 1500, and 1600 of respective FIGS. 24-26 may include suitable retention systems (see e.g., the retention system shown in FIG. 28).

Referring to FIG. 24, carrier 1400 includes a body portion 1412 having first and second ends, and an outer shell portion 1406. The body portion 1412 includes a plurality of sleeves for receiving containers, such as bottles. The shell and body portion 1406 and 1412 may be perforated to allow for effective heat transfer. In the illustrated embodiment, the shell portion 1406 is substantially cylindrical and the first and second ends are substantially circular to allow the carrier 1400 to roll. Referring now to FIGS. 25 and 26, the respective carriers 1500 and 1600 are substantially similar to the carrier 1400 of FIG. 24, but include different sized or shaped sleeves for receiving, for example, tubs and laminate containers. Any of the carriers 1400, 1500, and 1600 of respective FIGS. 24-26 may also be configured for receiving pouches.

As seen in FIG. 27, the body portions 1412, 1512, and 1612 of the respective bottle, tub, and laminate container carriers 1400, 1500, and 1600 may each be interchangeable within a single outer shell portion 1406. Such interchangeability of parts allows for various types of containers to be processed using the carrier system, as well as replacement of worn parts without requiring replacement of the entire carrier.

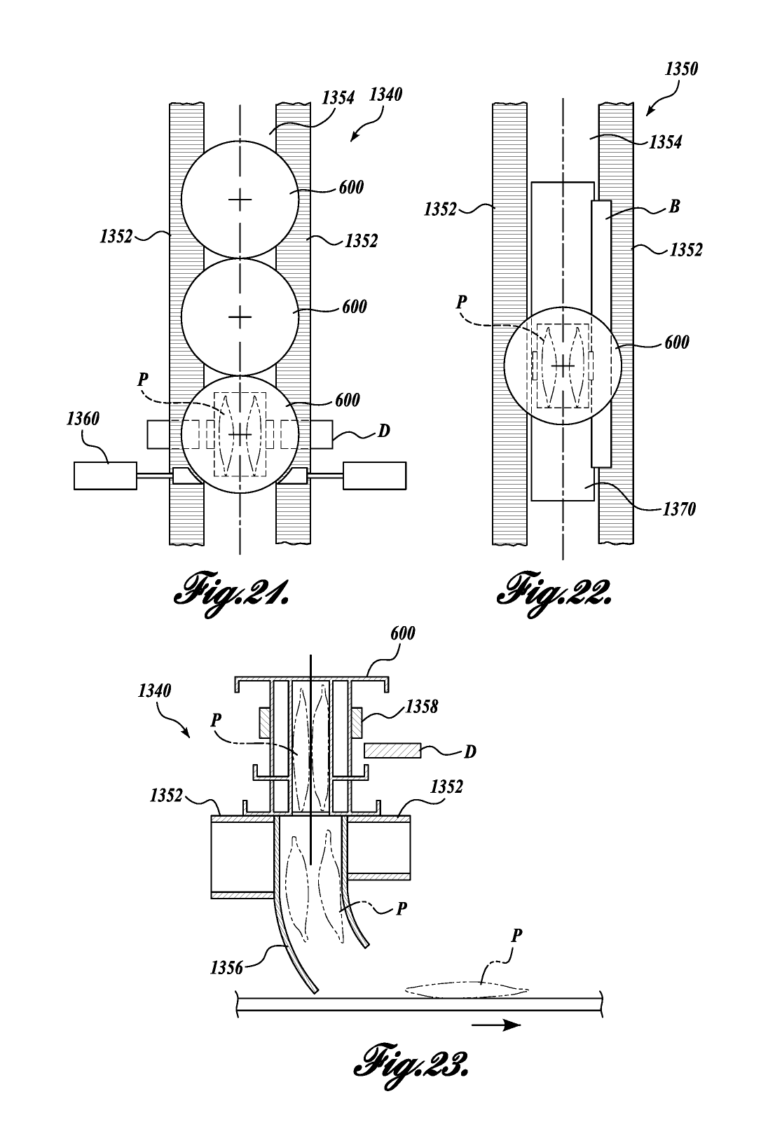

Referring now to FIGS. 28 and 29, alternate embodiments for carrier assemblies are provided. The carriers 1700 and 1800 are substantially cylindrical in shape, like the previously described embodiments; however, they have differences regarding the positioning and orientation of the sleeves 1710 and 1810 for carrying pouches P. For example, as shown in FIG. 28, the sleeves 1710 are positioned to extend radially from the center longitudinal axis of the container 1700. An optional circular-shaped capping portion 1714 having a plurality of holes 1712 can be fixed to the top surface 1716 of the container 1700. After pouches P have been received in the sleeves 1710 through aligned holes 1712 in the capping portion 1714, the capping portion 1714 may be turned relative to the container 1700 and sleeves 1710 to misalign the holes 1712 in the capping portion 1714 with the sleeves 1710. Such misalignment helps maintain the pouches P in the sleeves 1710 during processing.

Referring now to FIG. 29, the sleeves 1810 are also positioned to extend radially from the center longitudinal axis of the container 1800. However, different from the embodiment shown in FIG. 28, the sleeves 1810 are accessed from the outer cylindrical surface 1816 of the container 1800. An optional cylindrical capping portion 1814 having a plurality of holes 1812 can be fixed to the outer cylindrical surface 1816 of the container 1800. After pouches P have been received in the sleeves 1810 through aligned holes 1812 in the capping portion 1814, the capping portion 1714 may be turned relative to the container 1800 and sleeves 1810 to misalign the holes 1812 in the capping portion 1814 with the sleeves 1810. Such misalignment helps maintain the pouches P in the sleeves 1810 during processing.

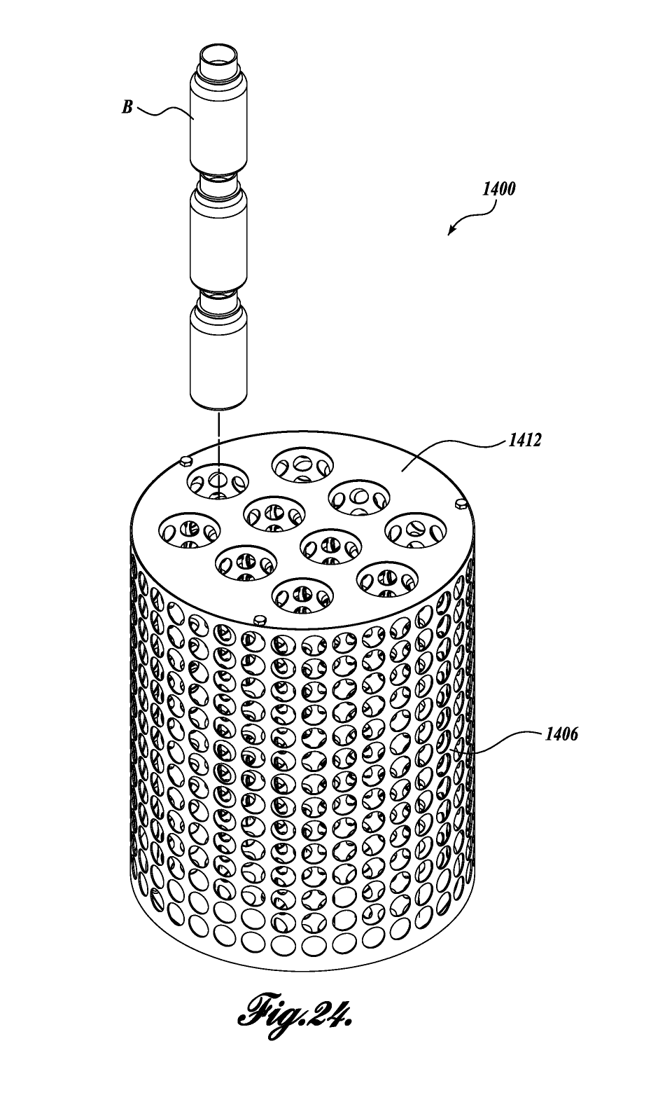

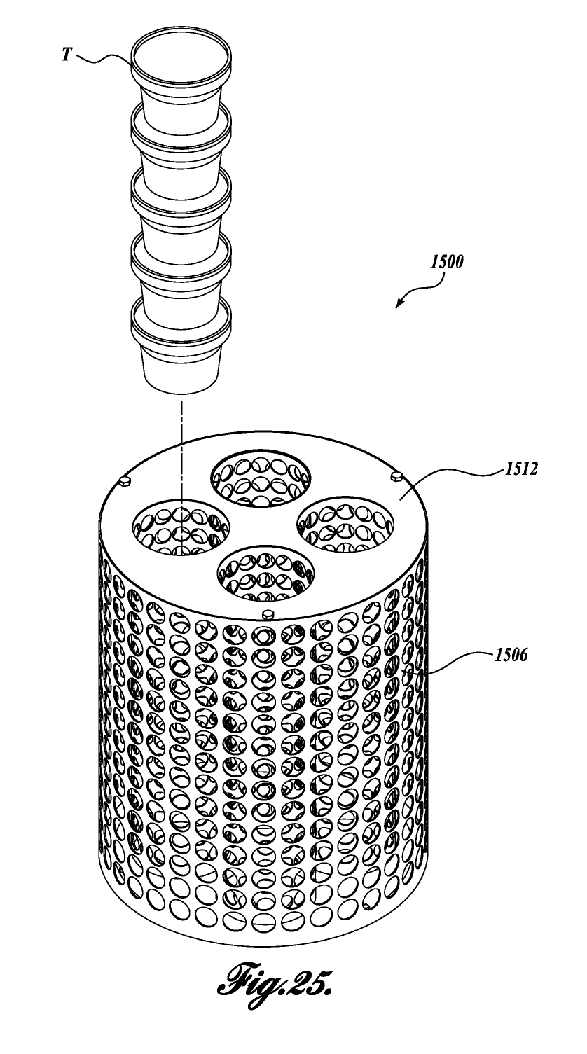

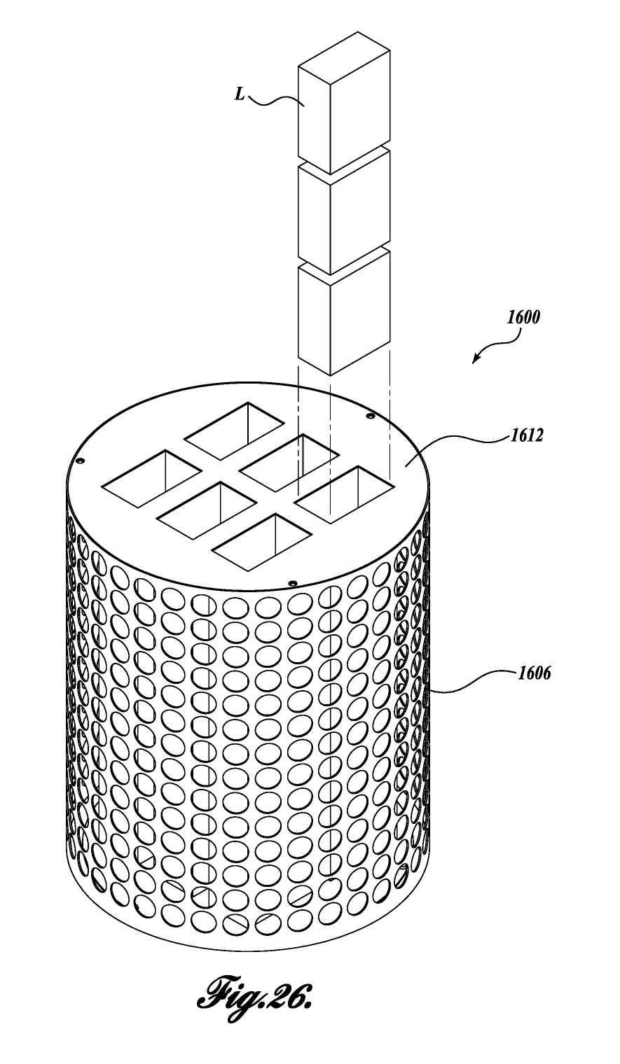

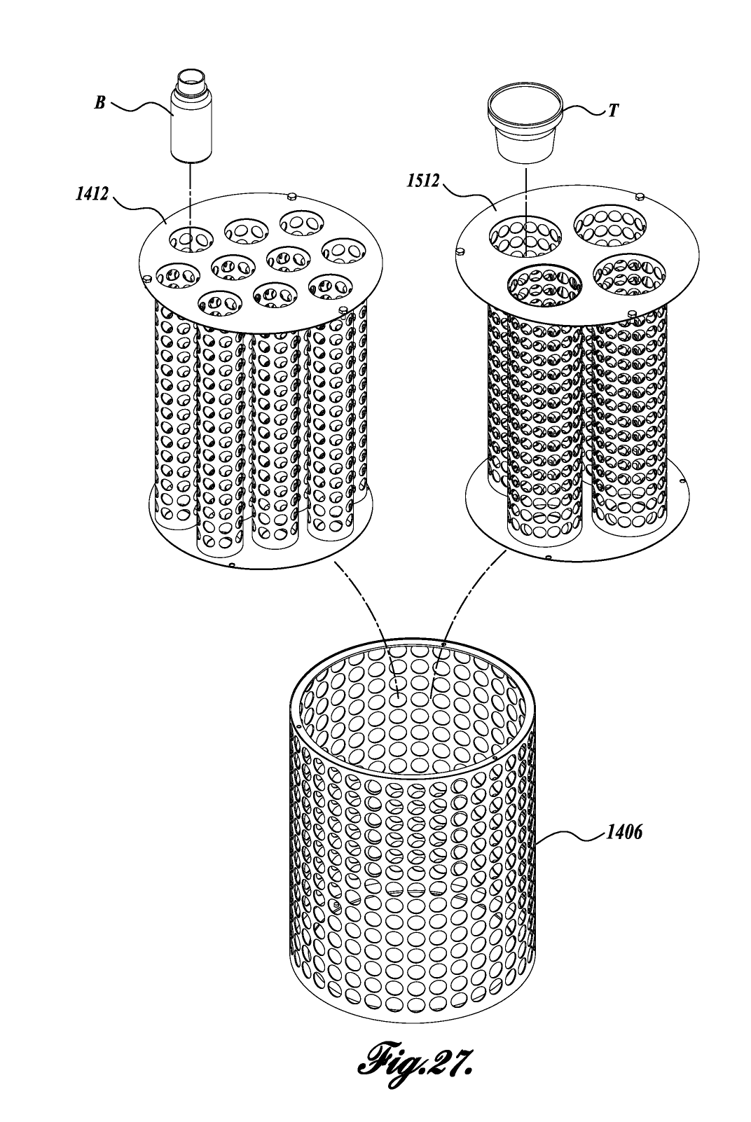

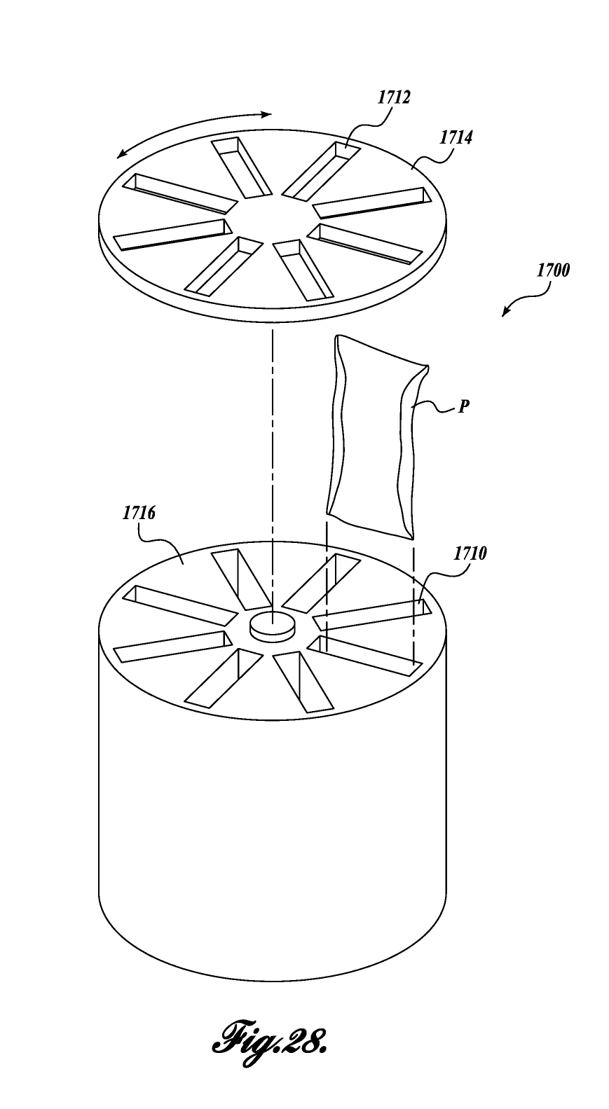

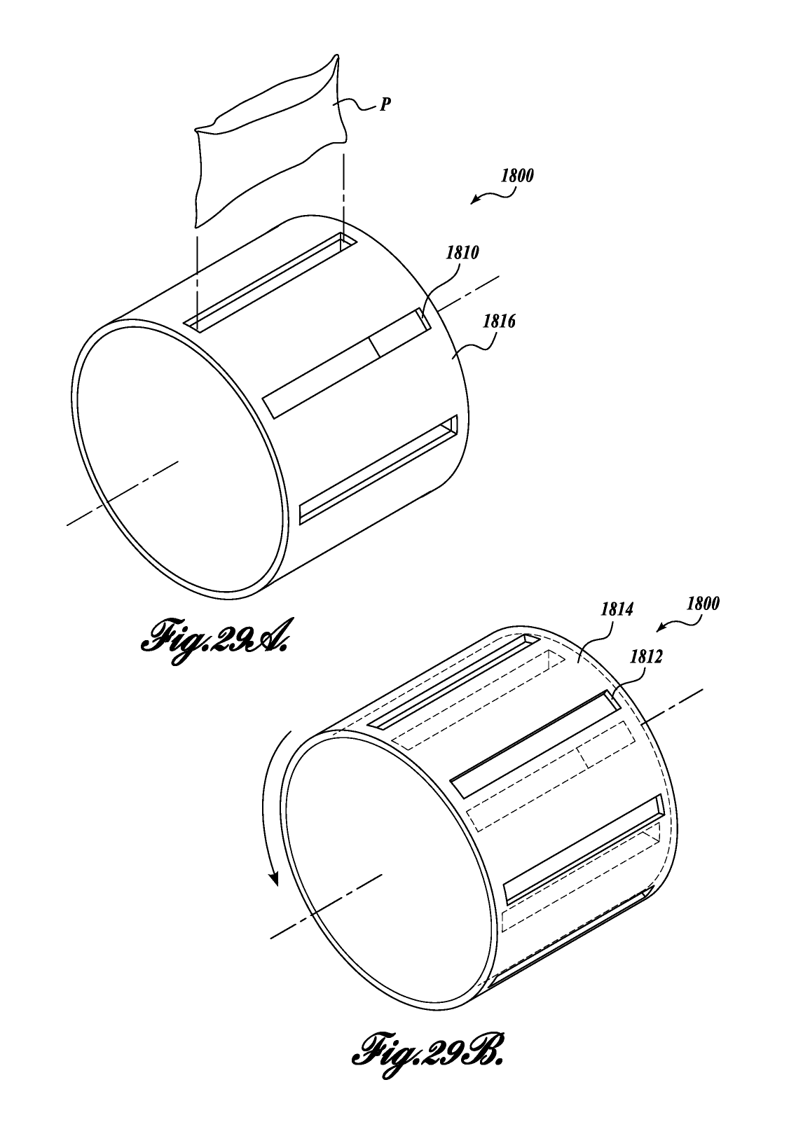

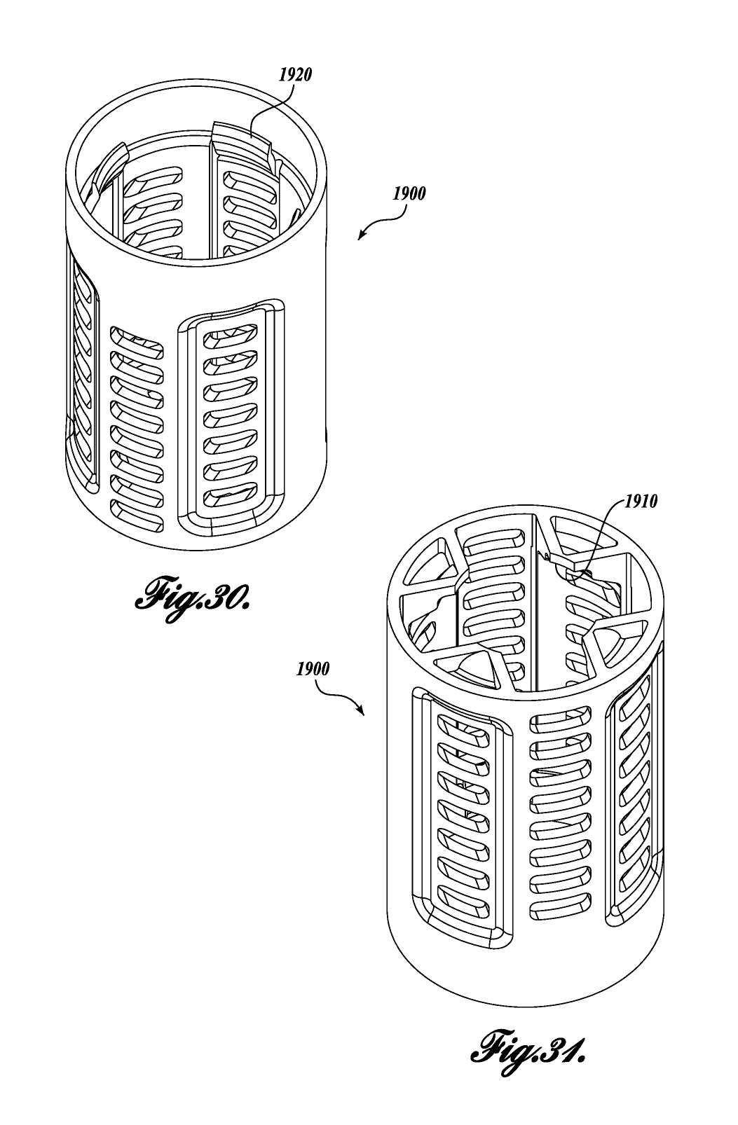

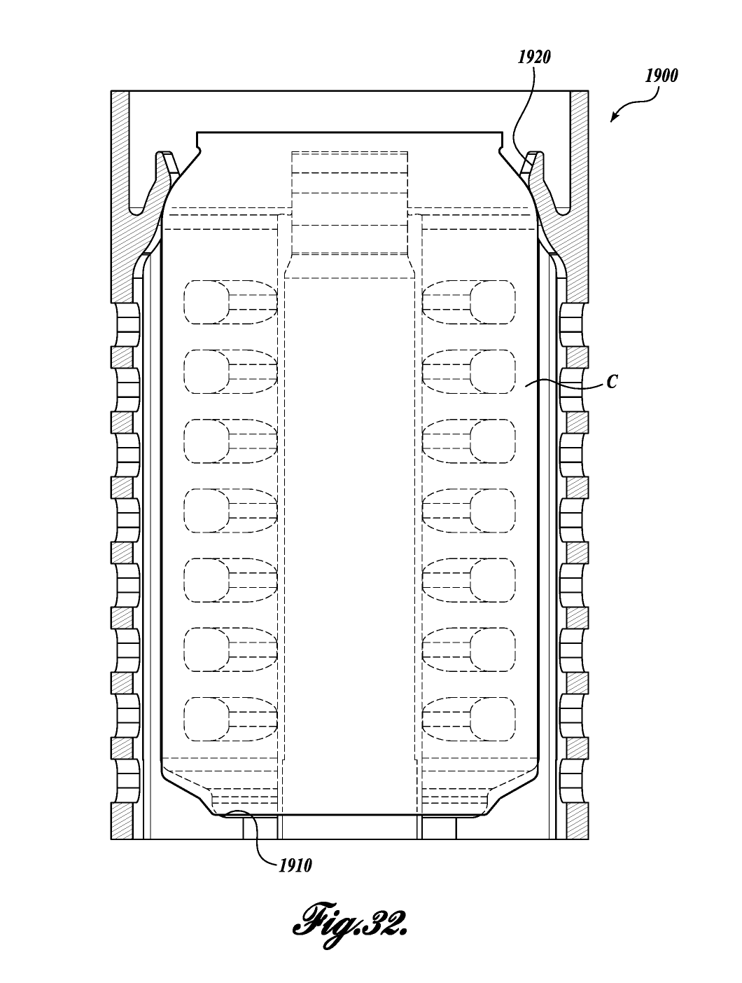

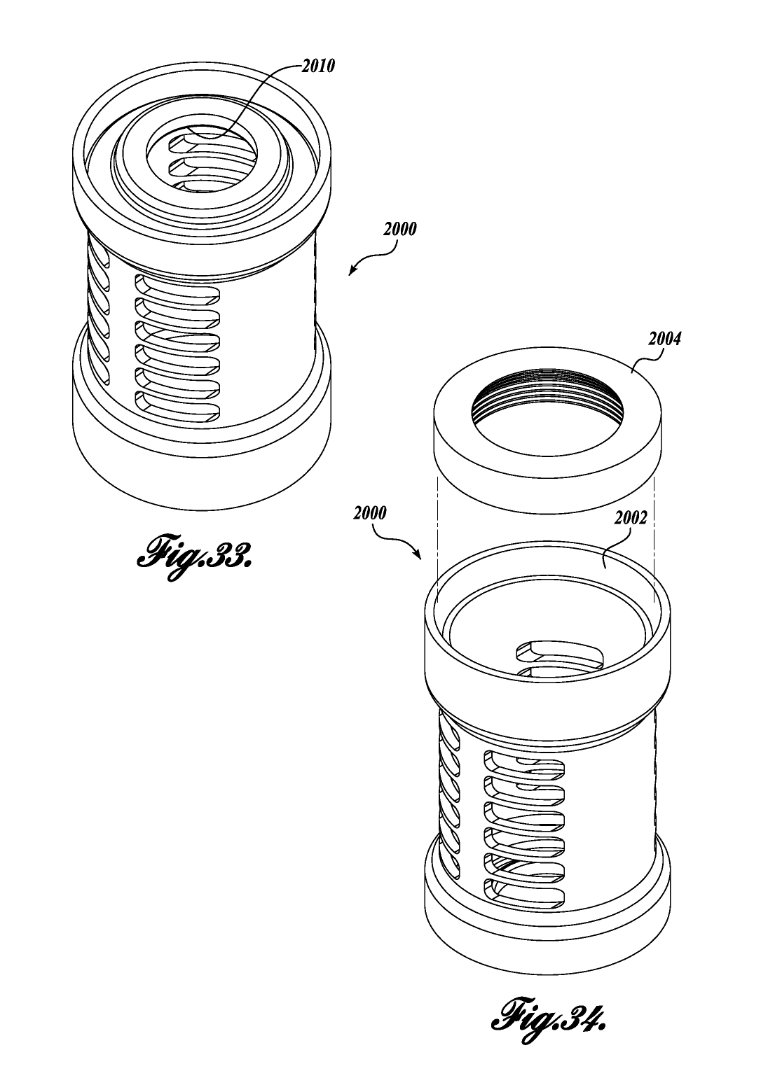

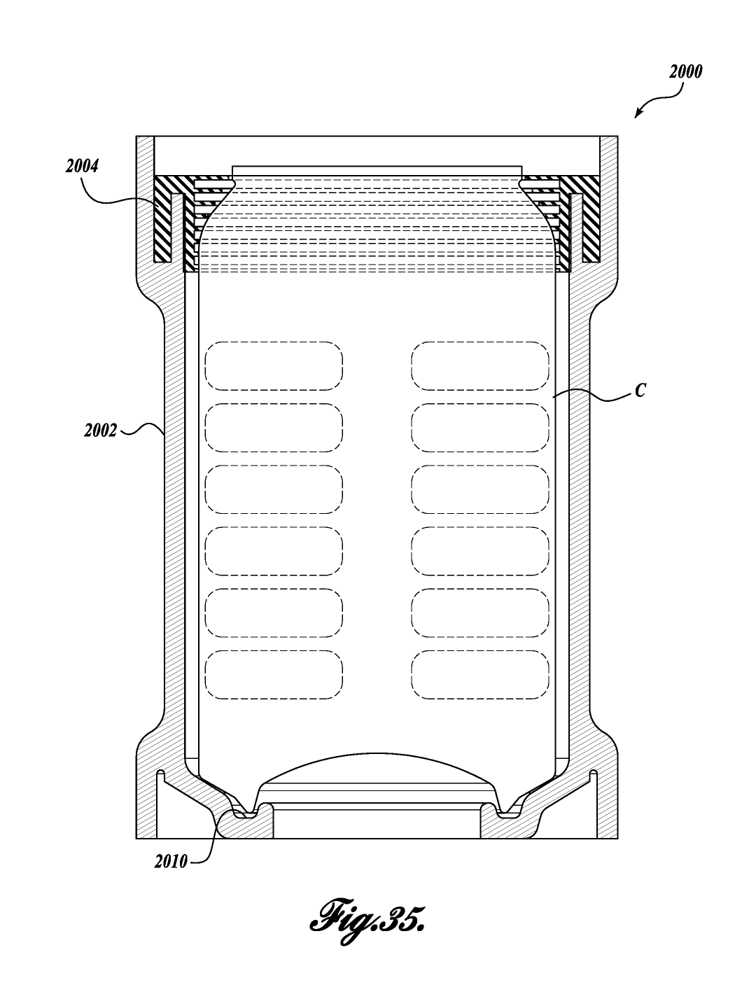

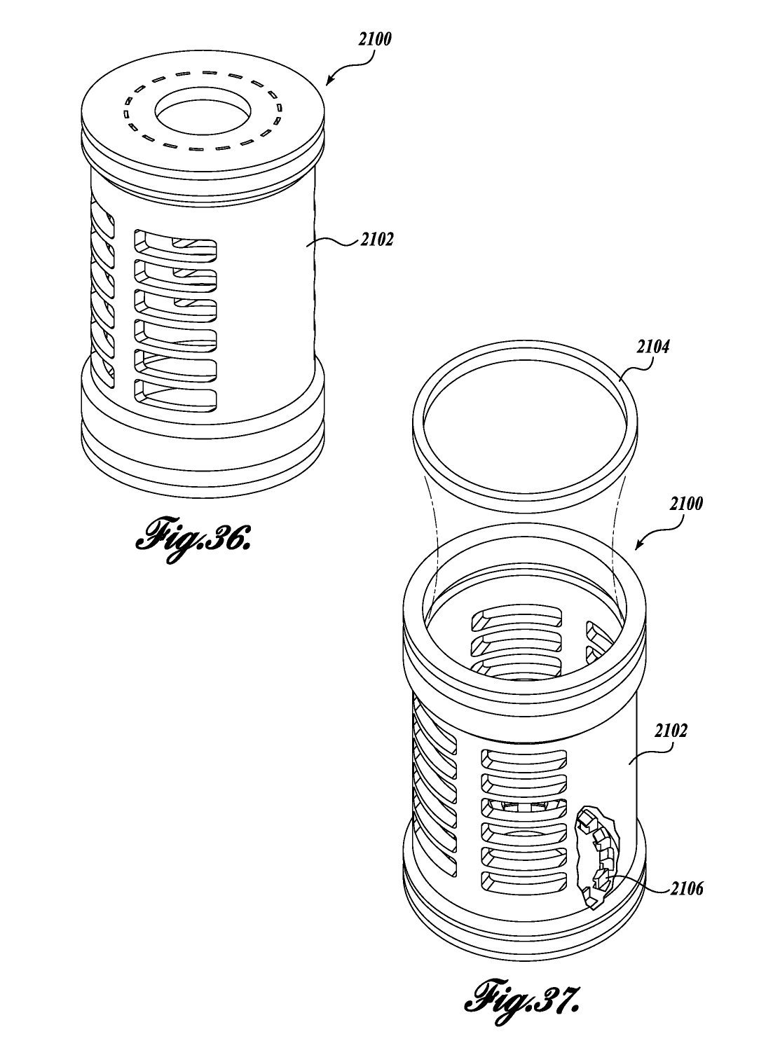

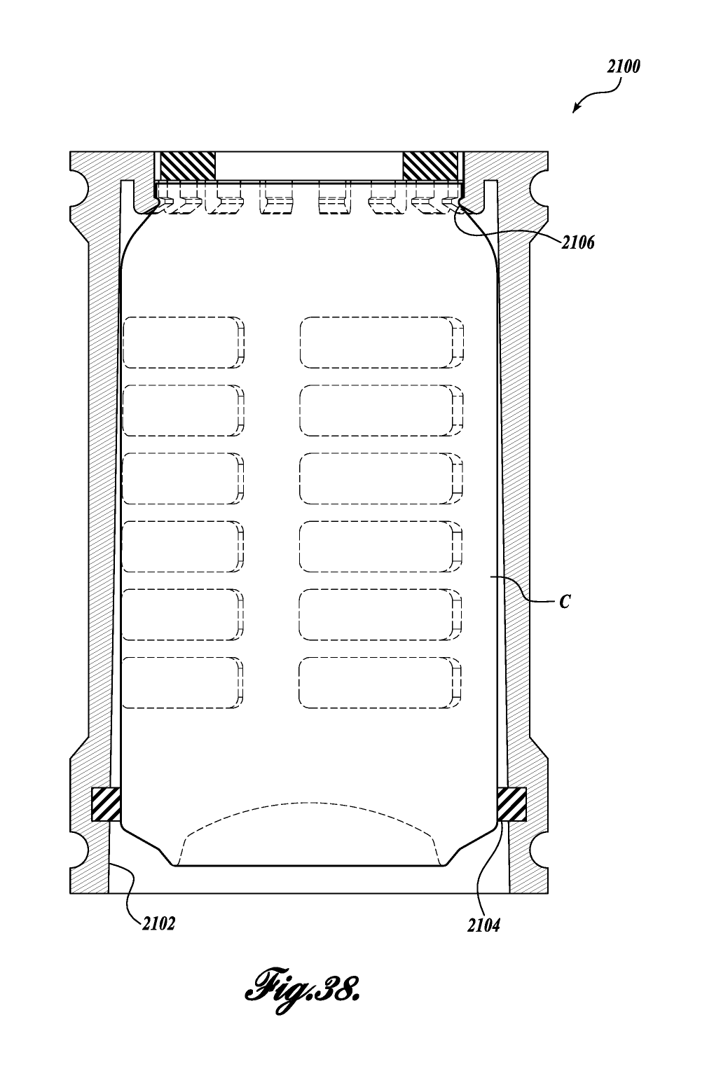

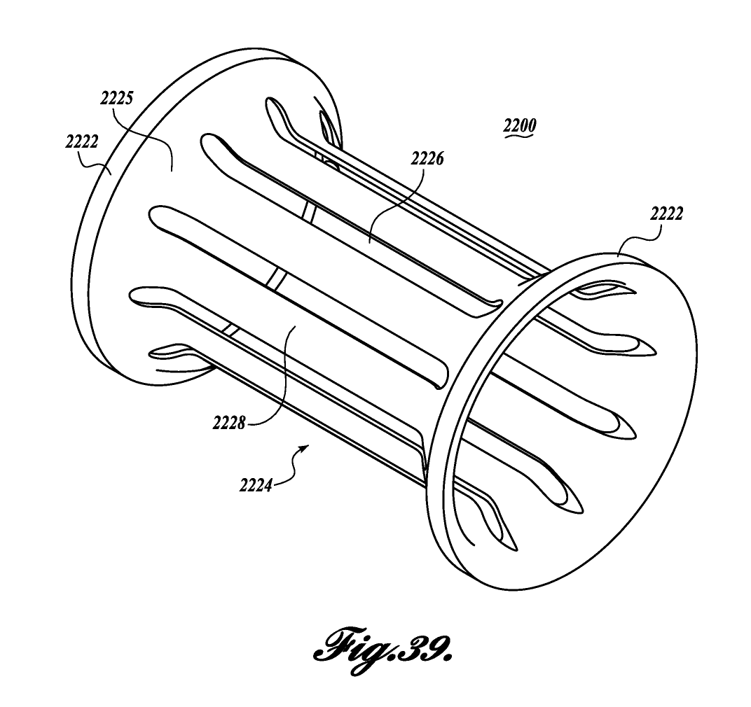

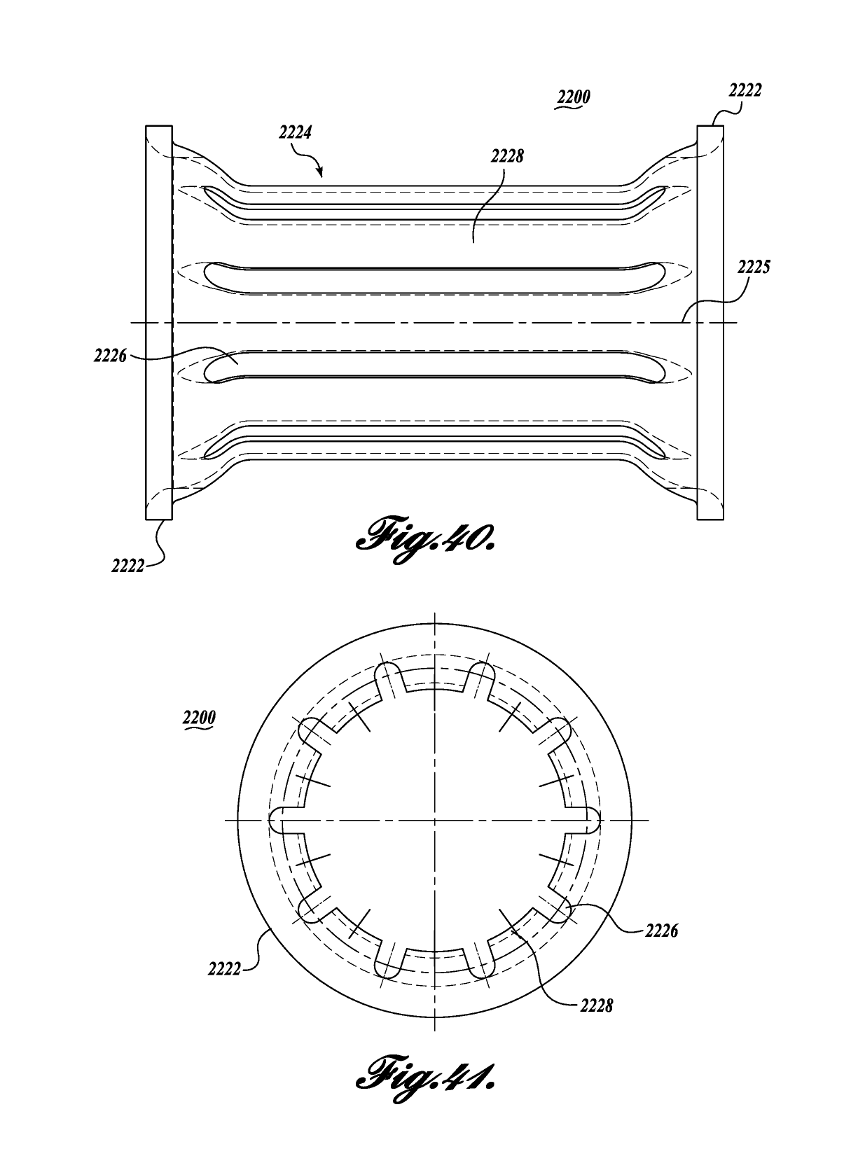

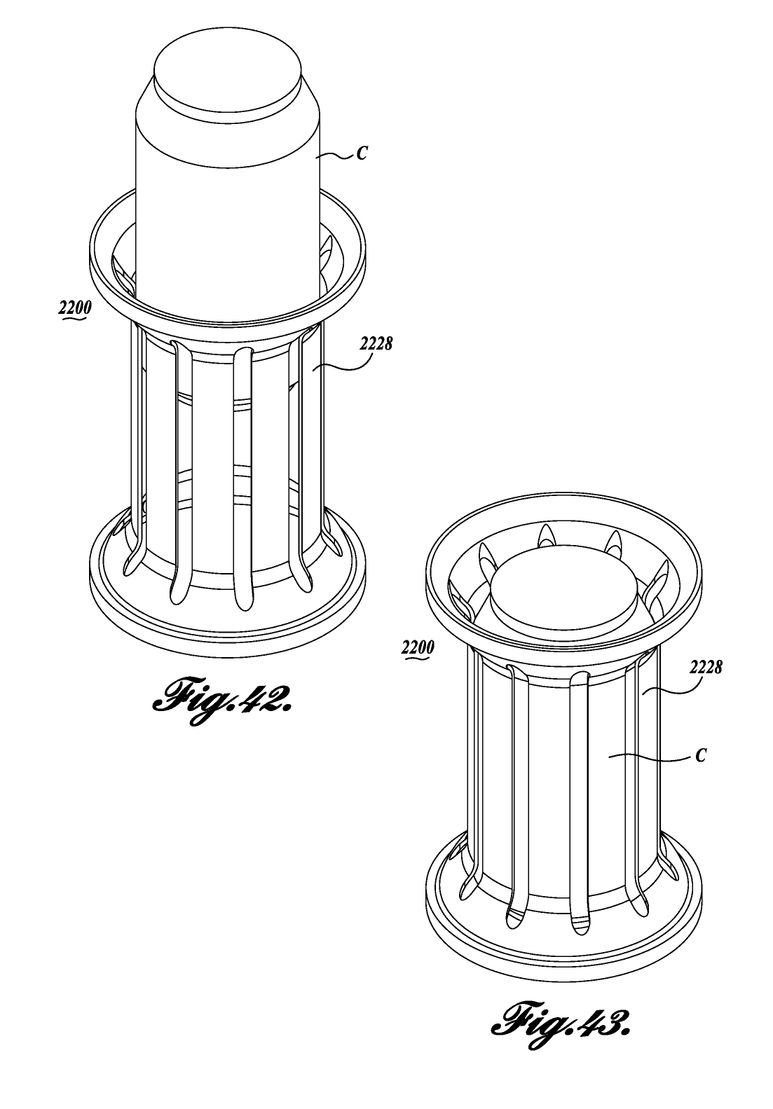

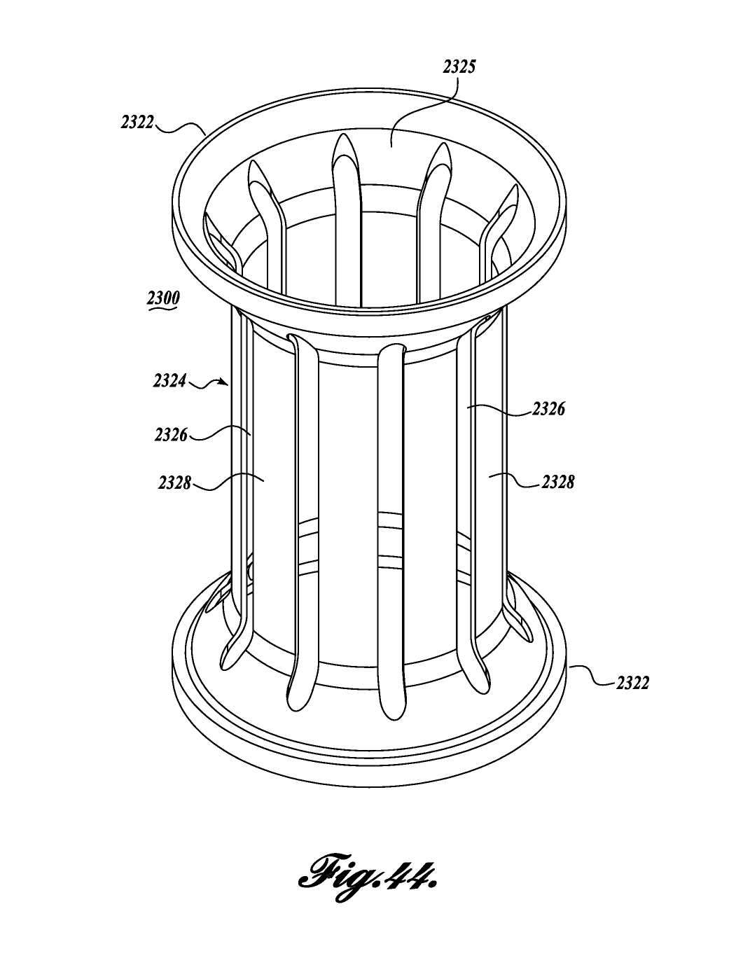

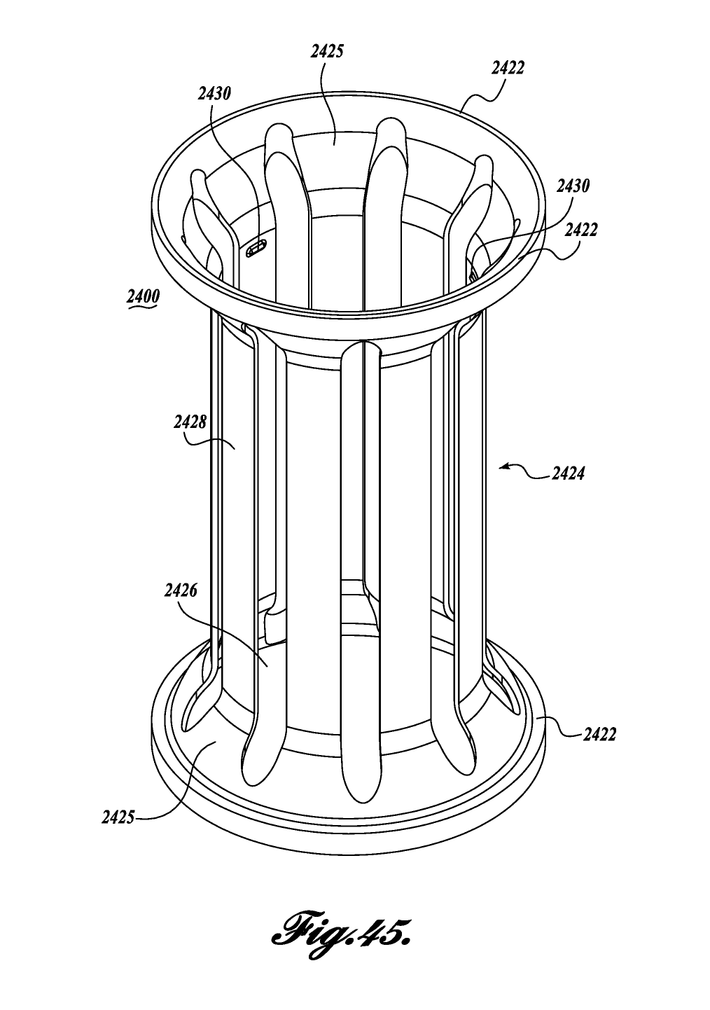

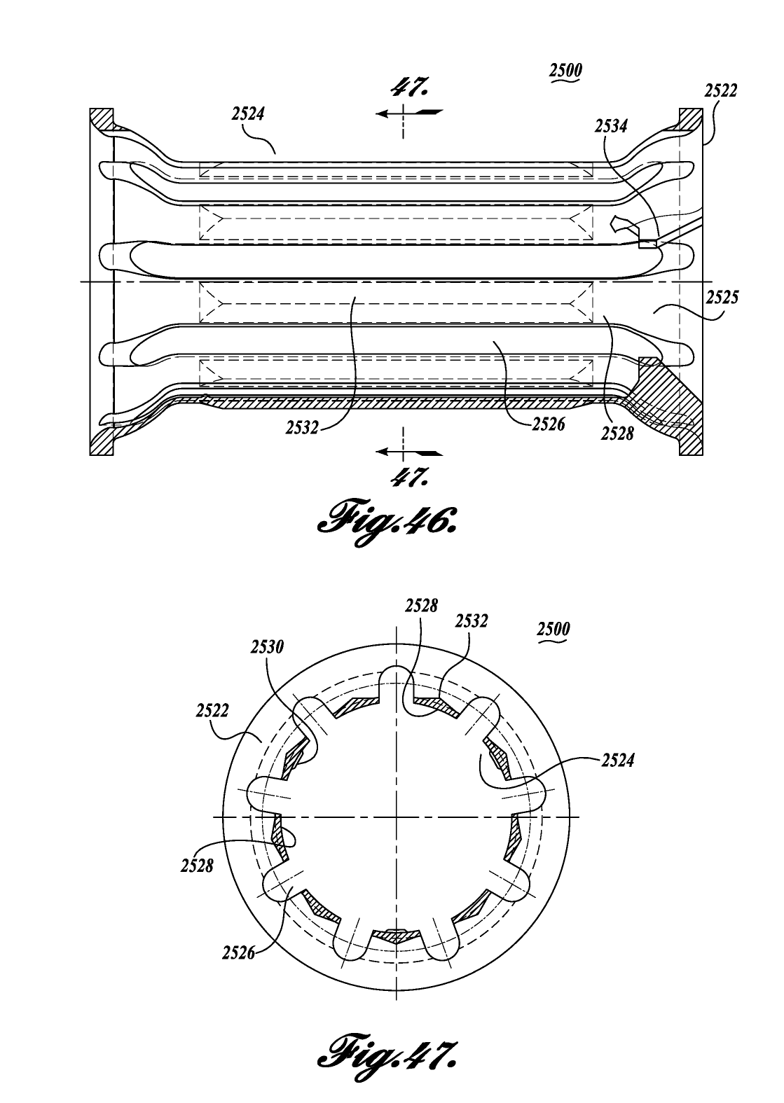

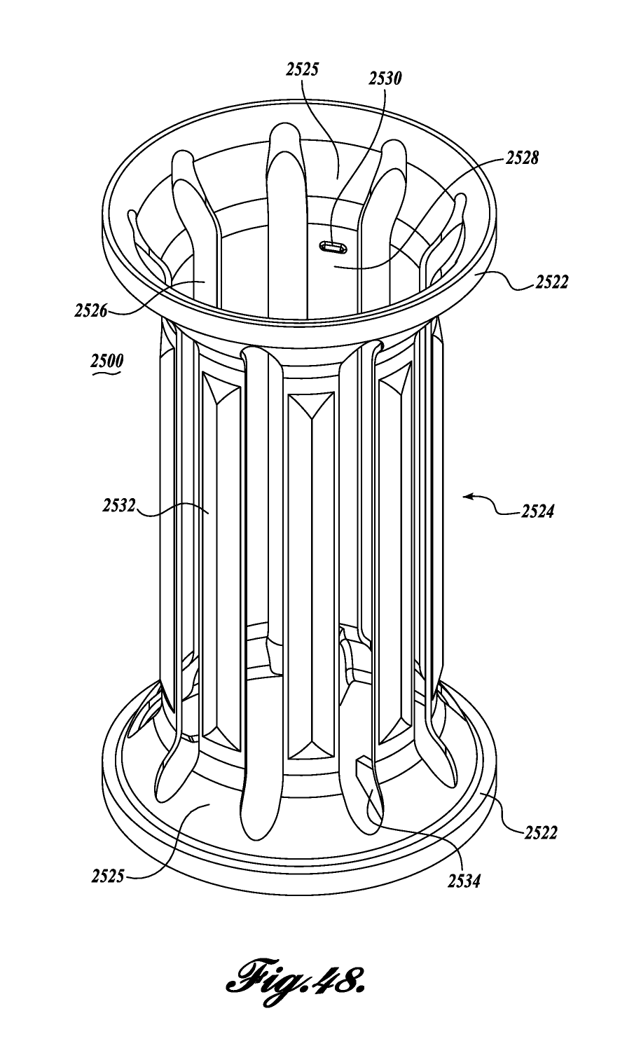

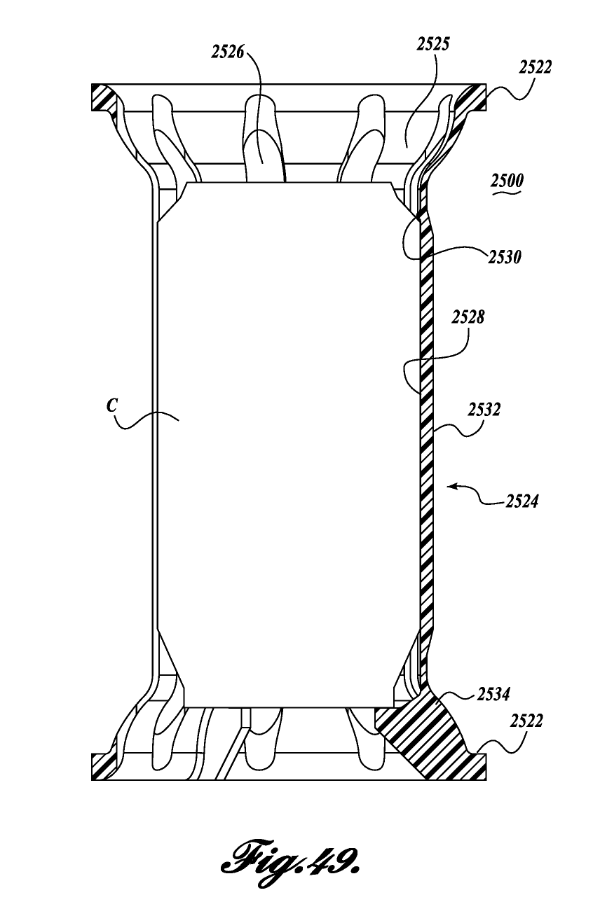

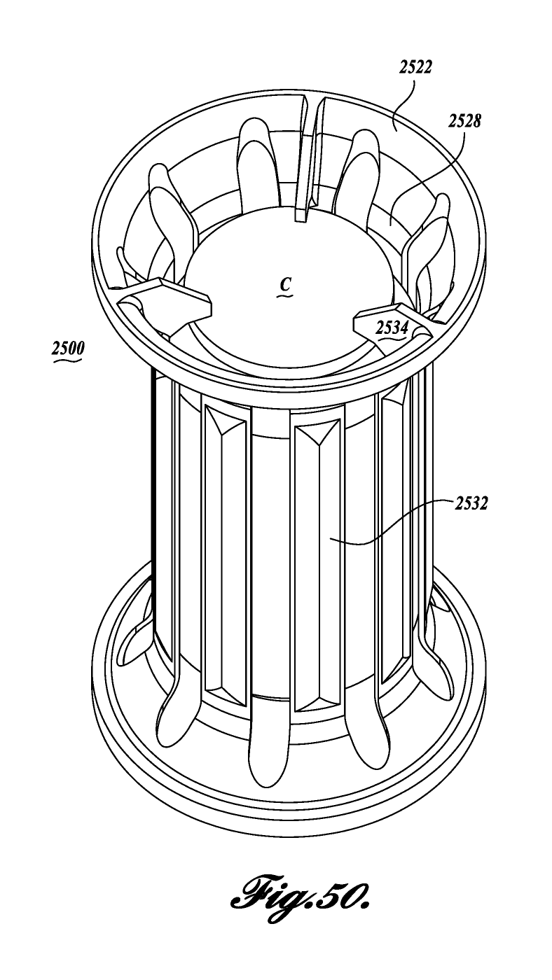

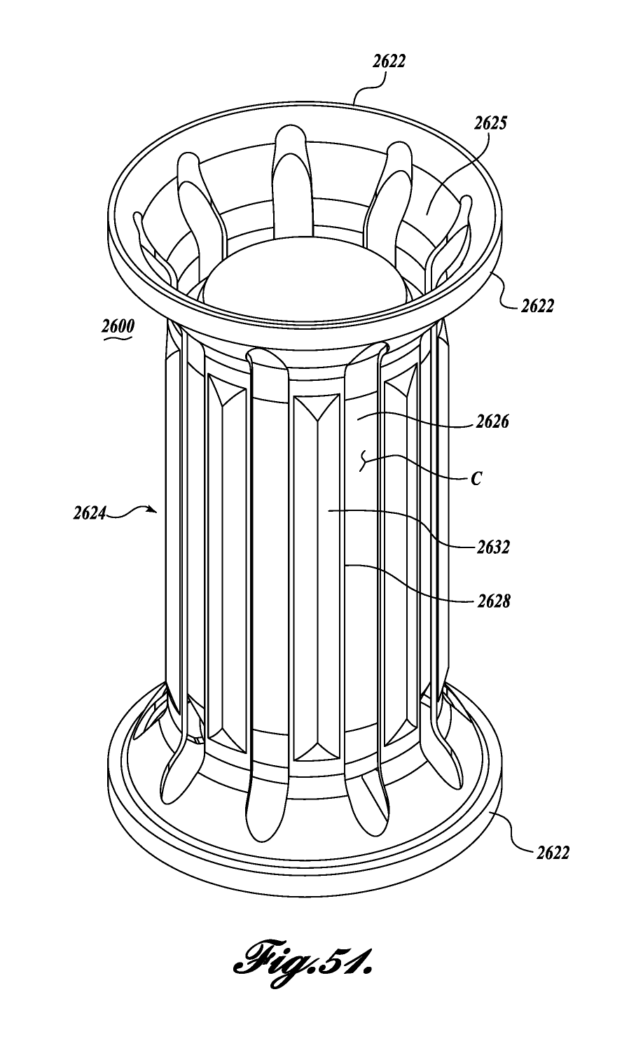

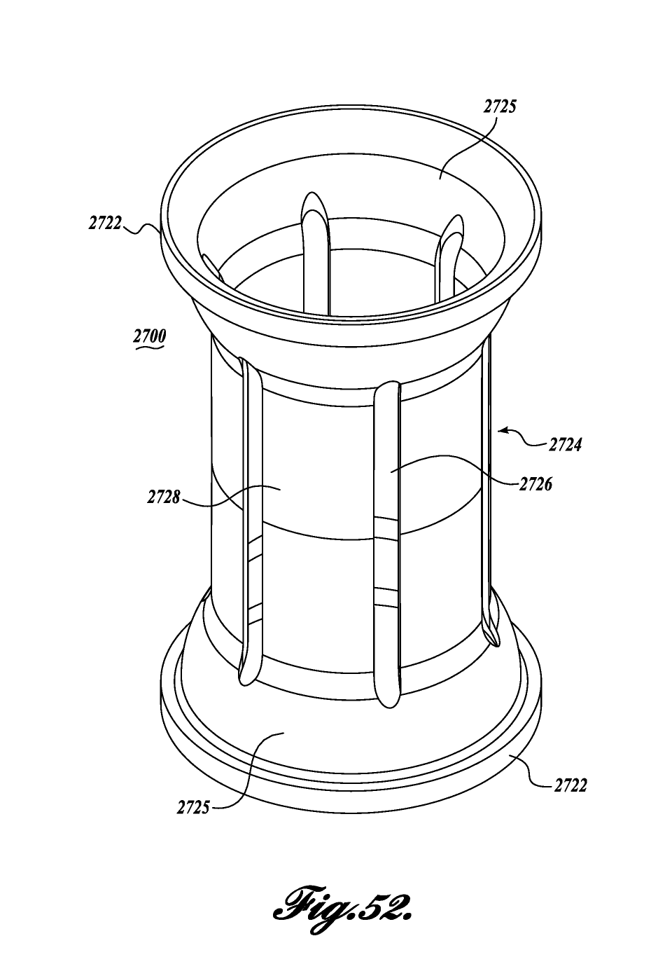

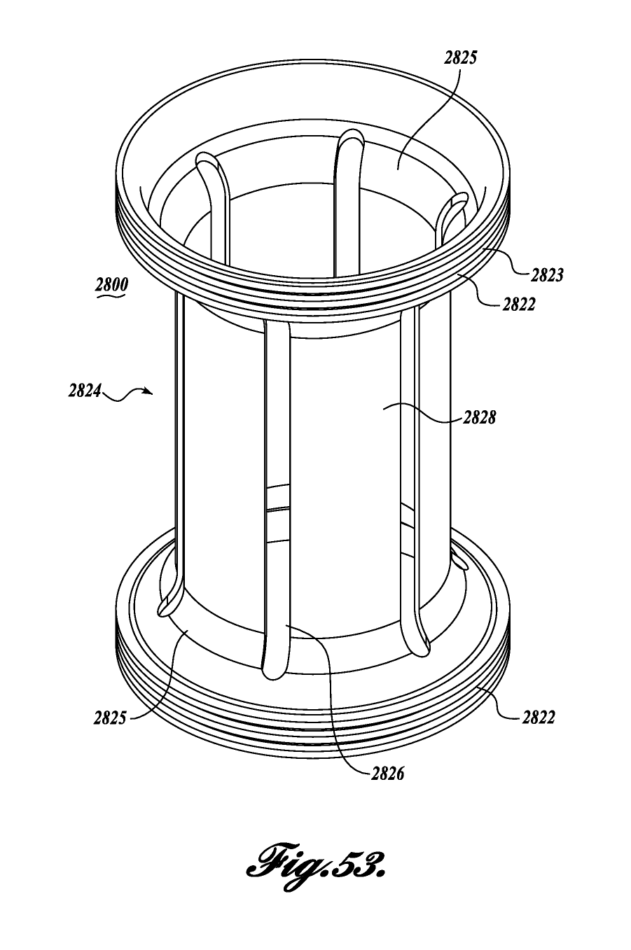

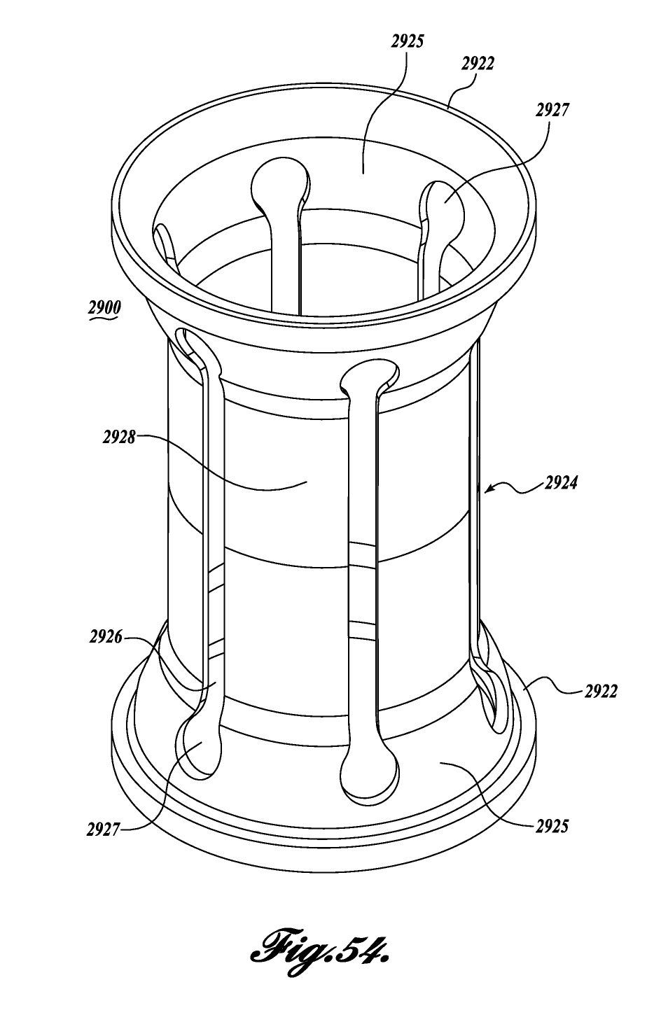

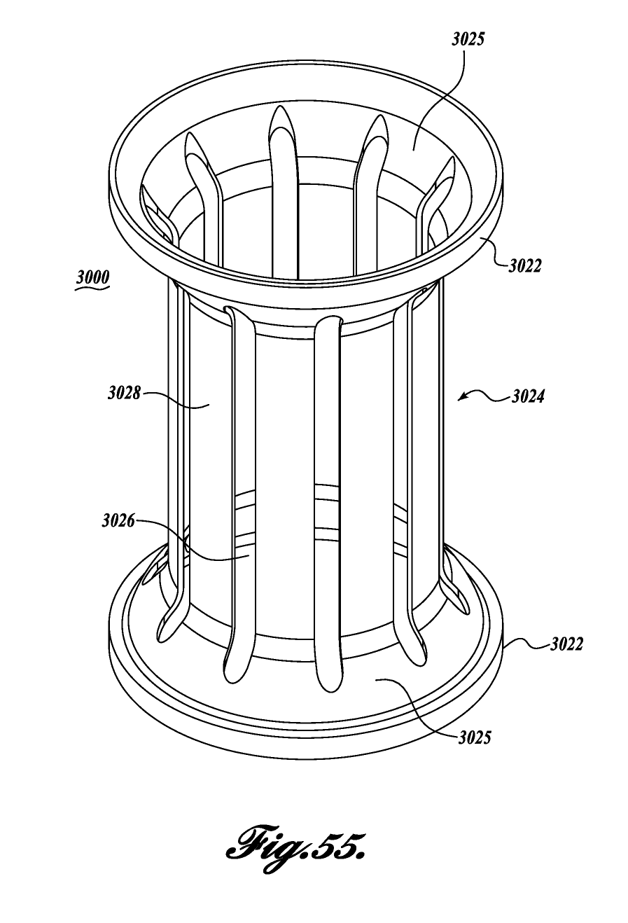

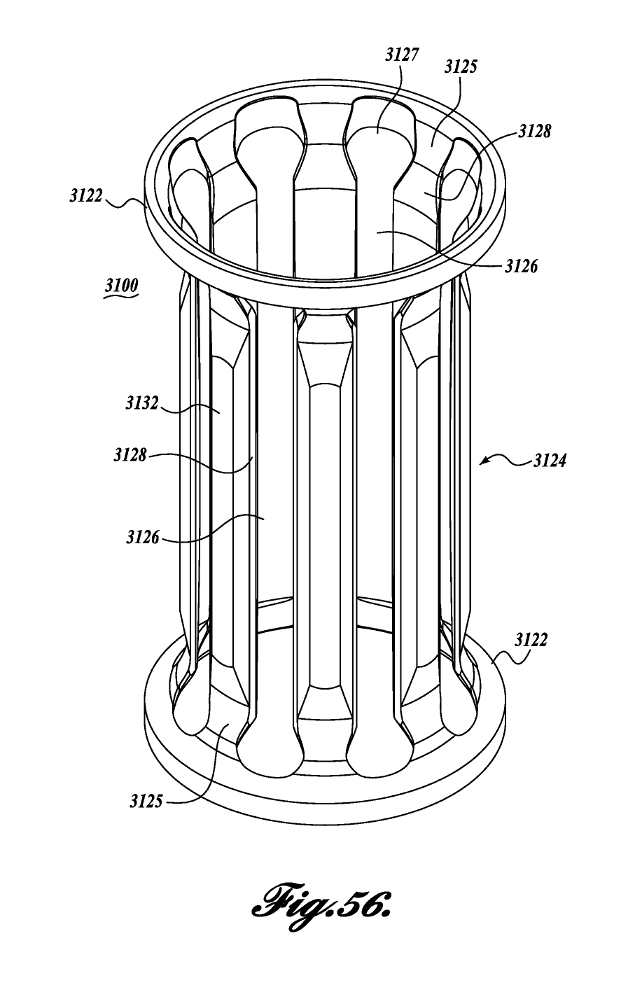

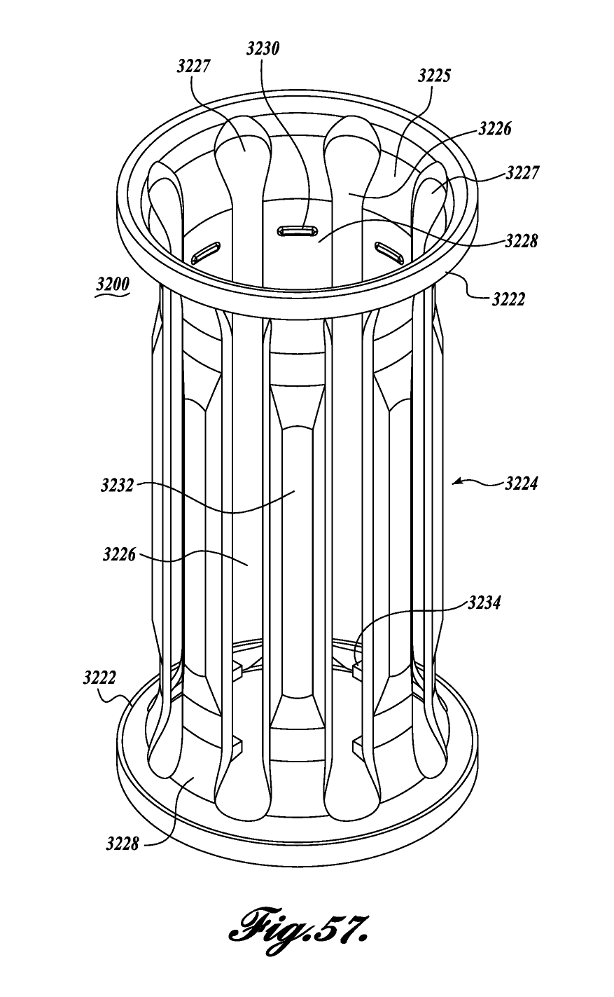

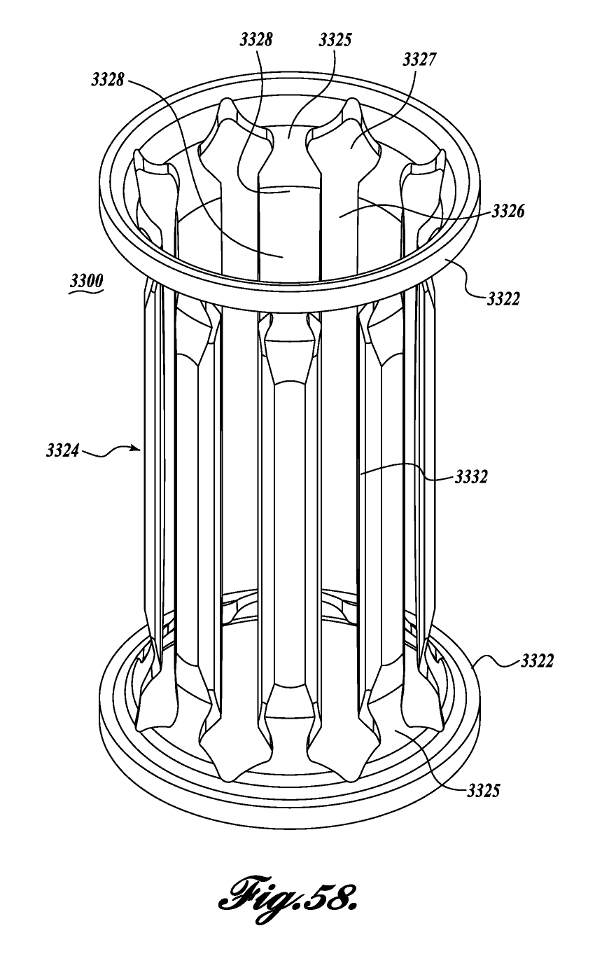

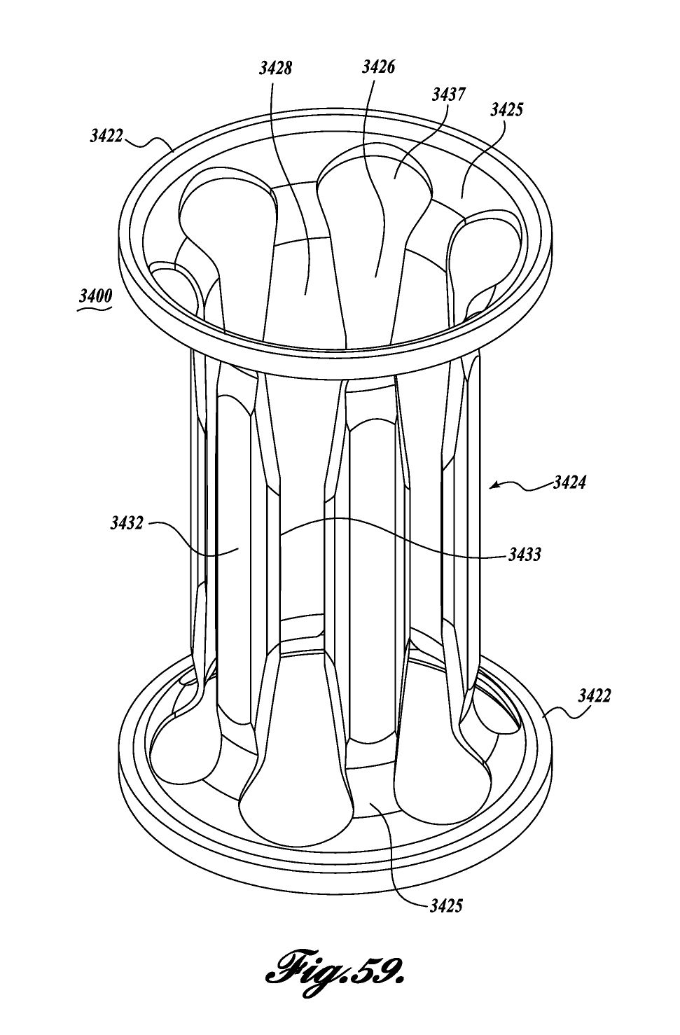

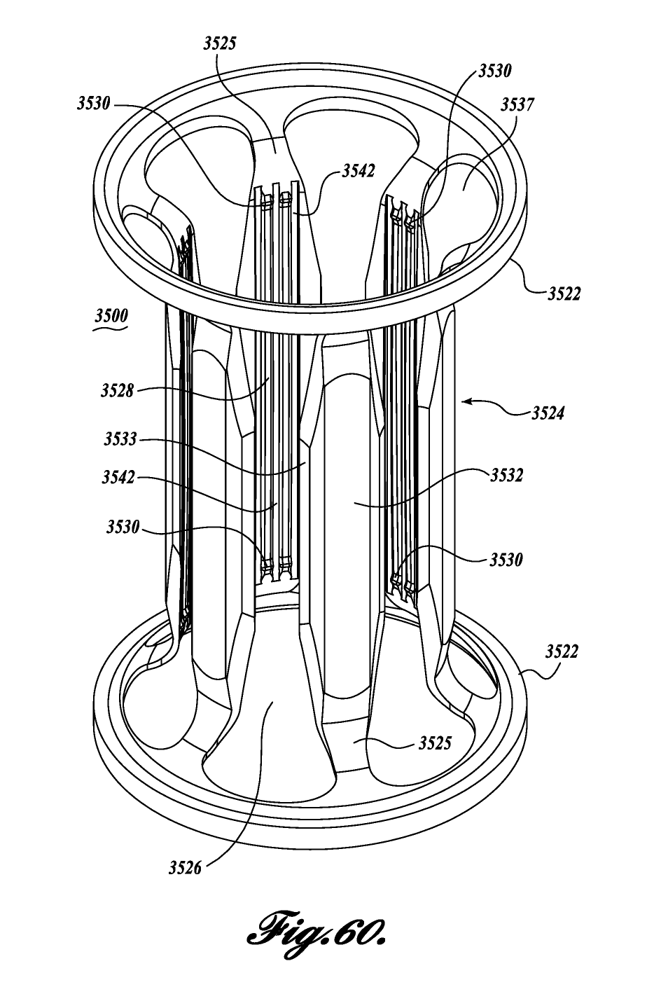

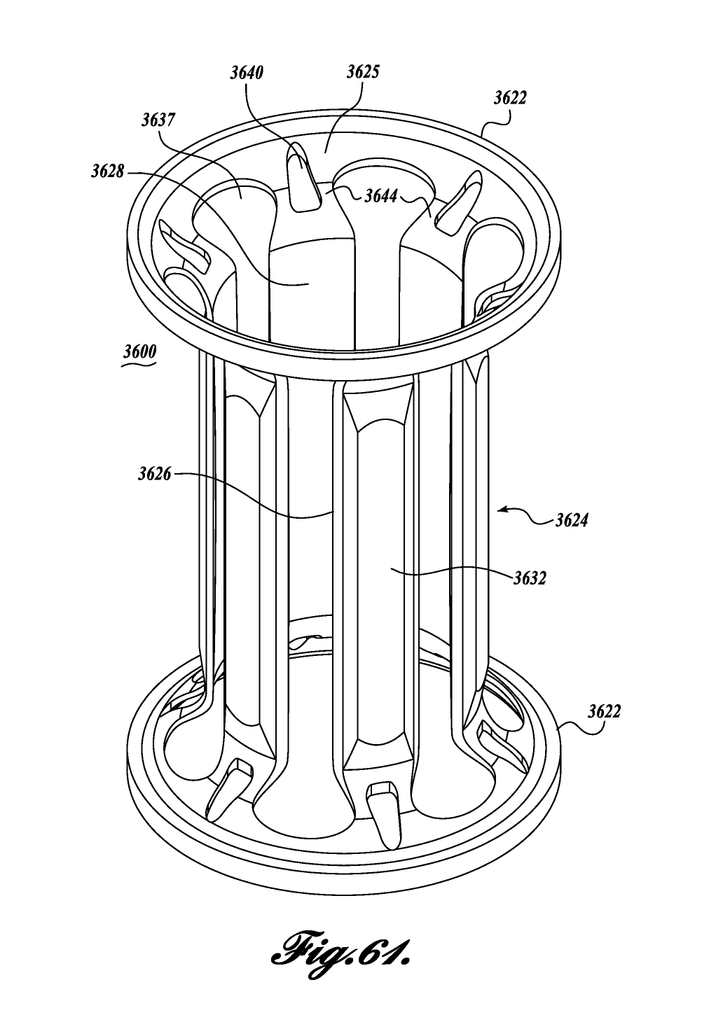

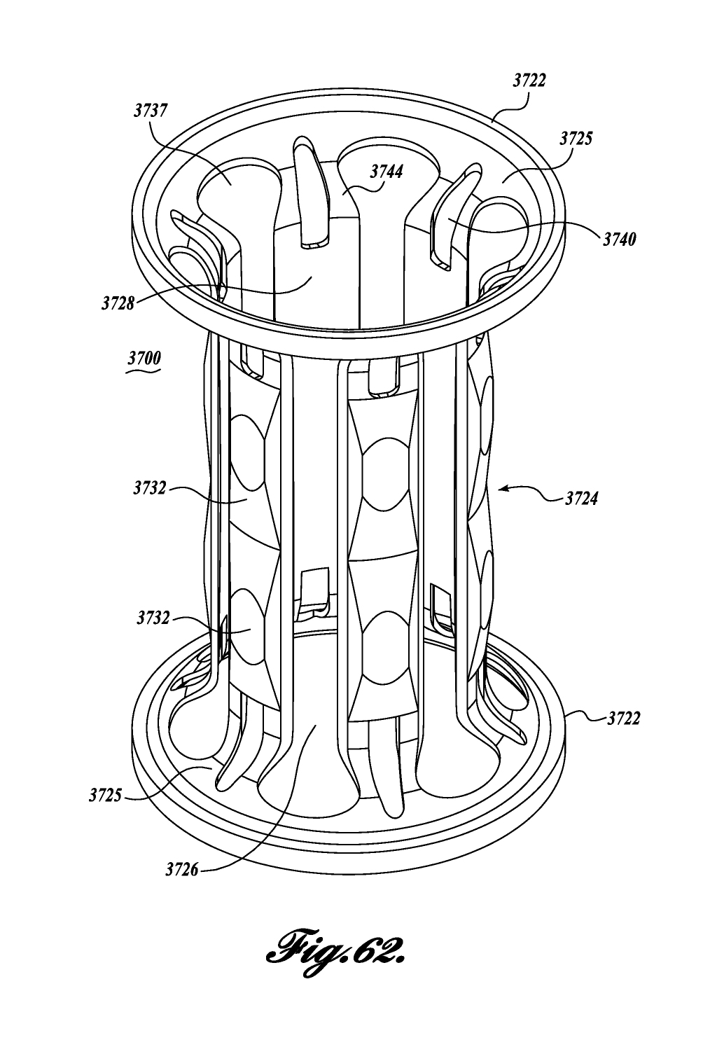

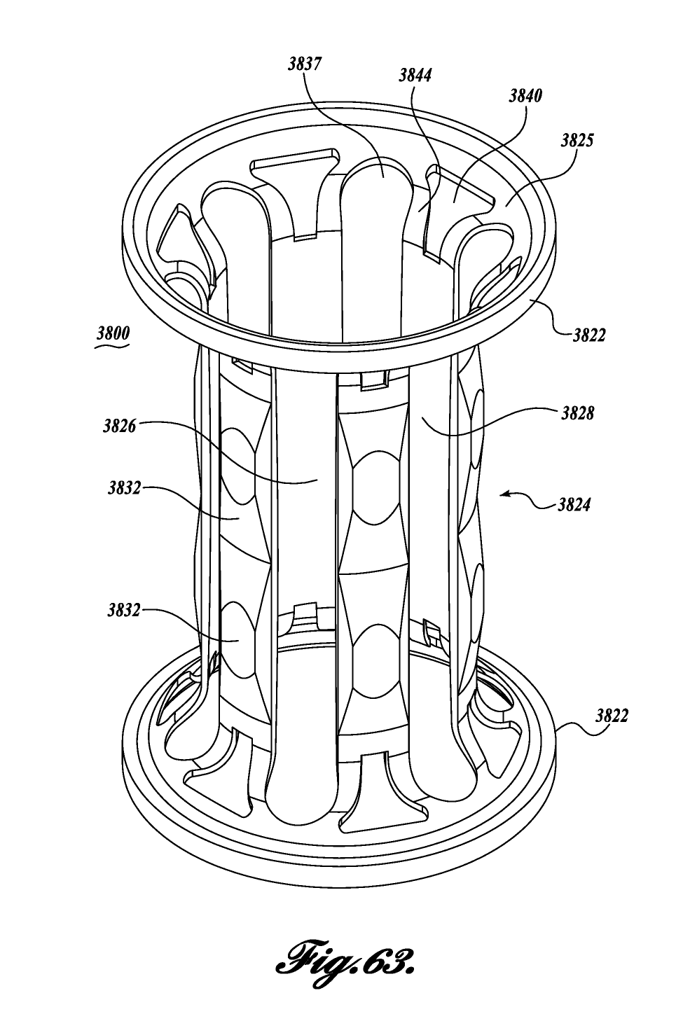

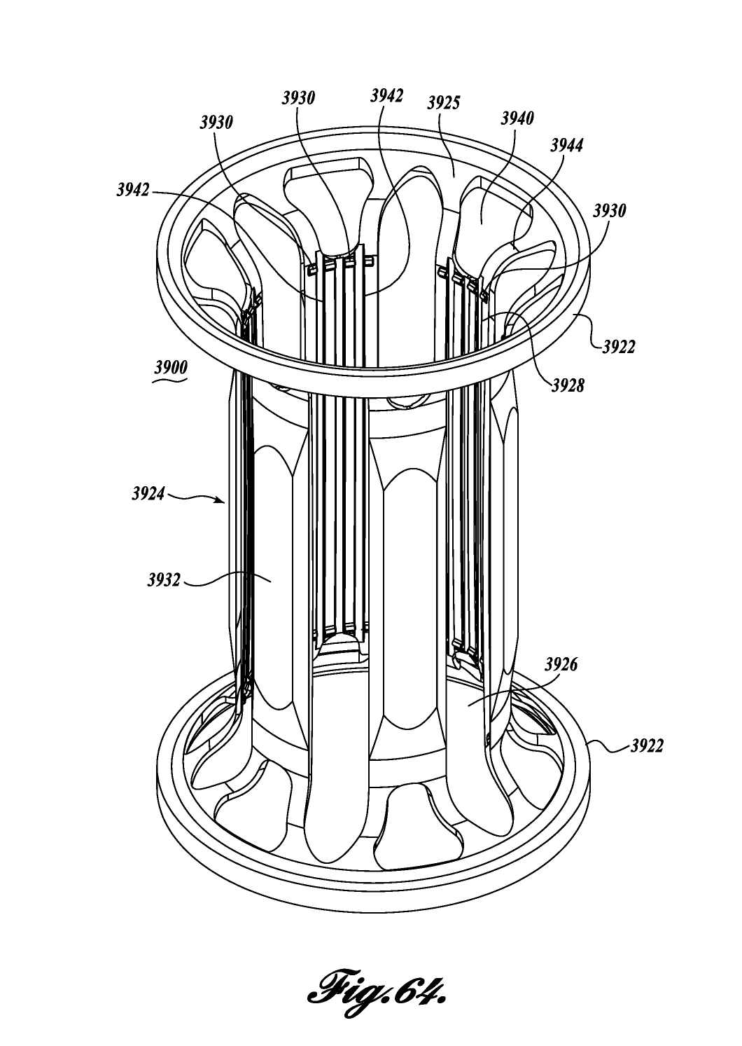

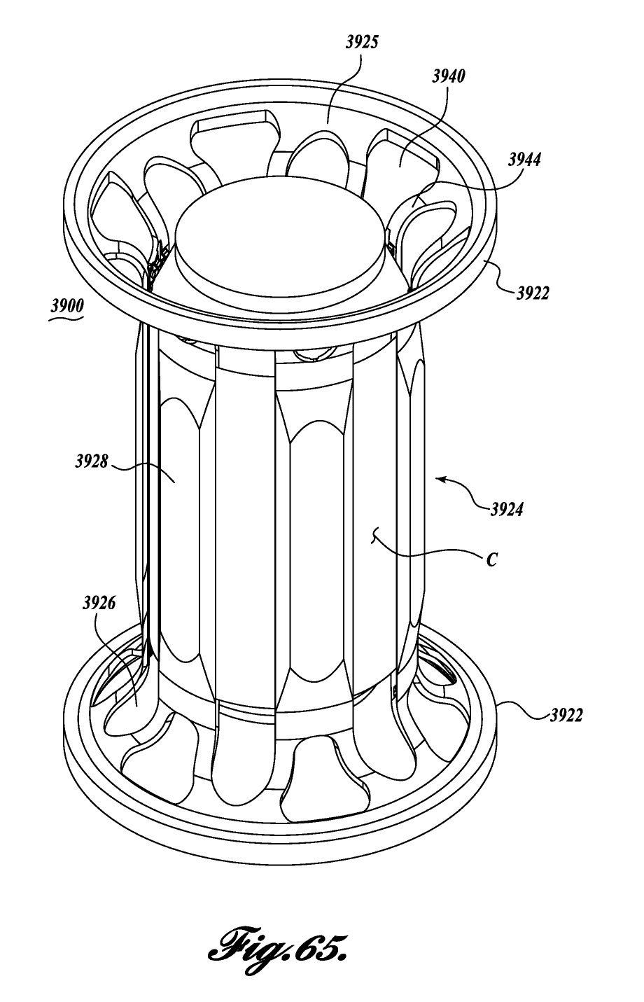

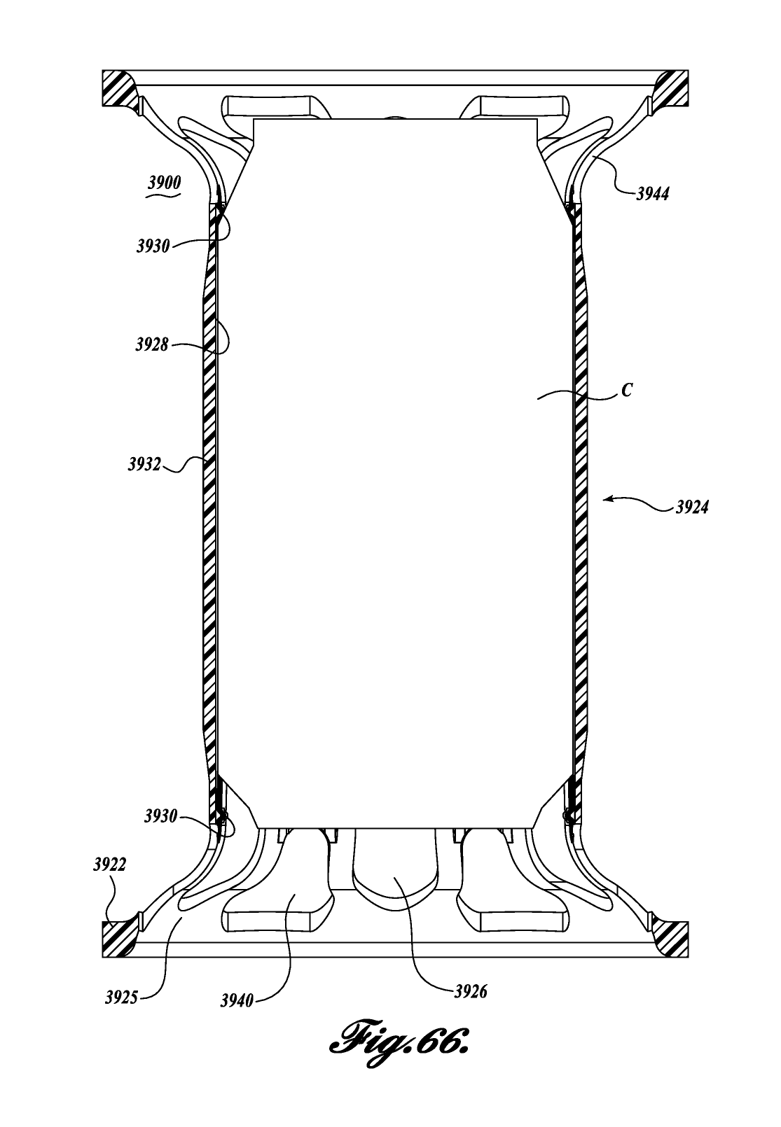

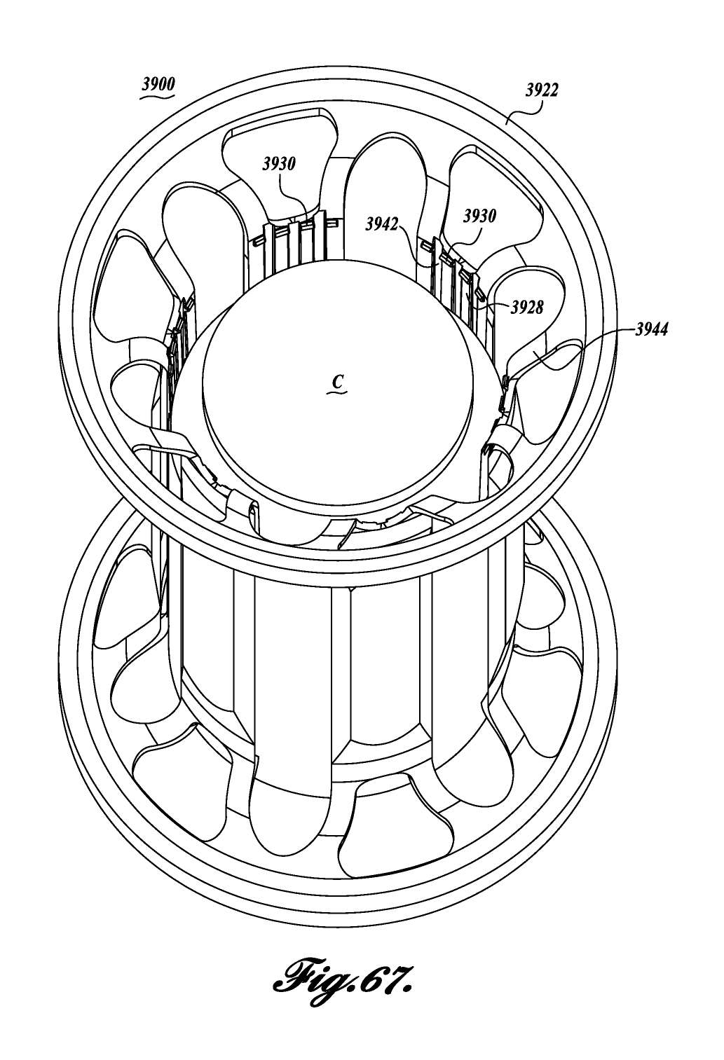

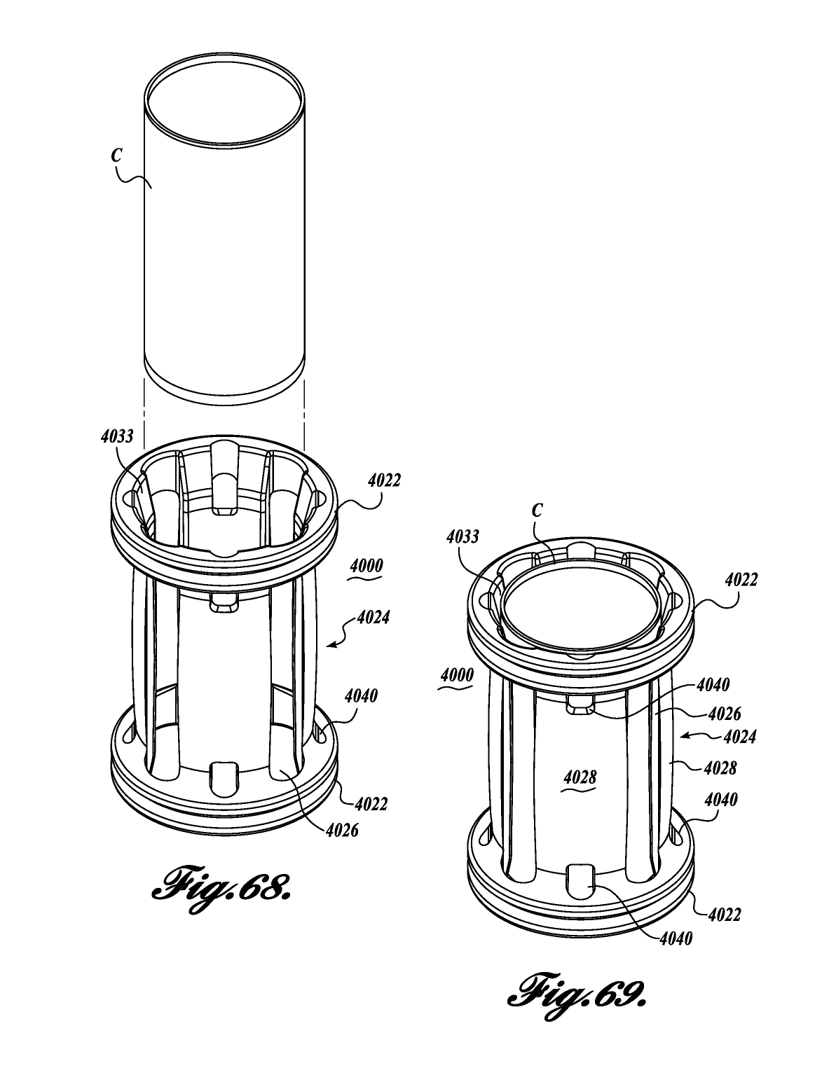

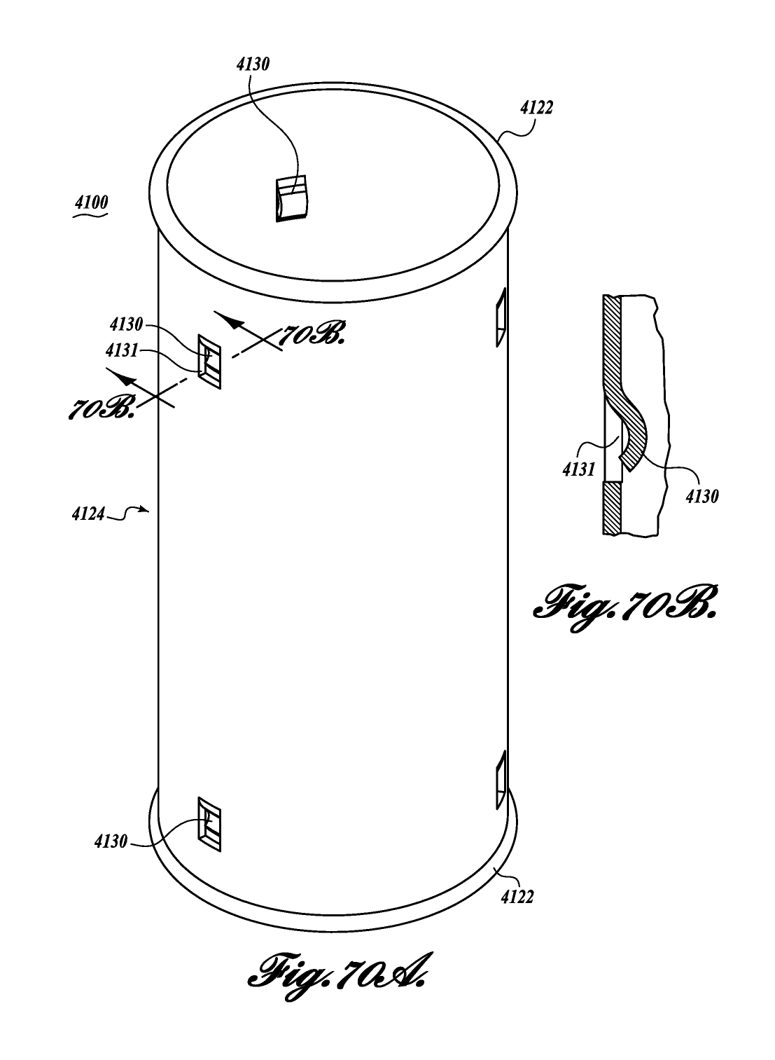

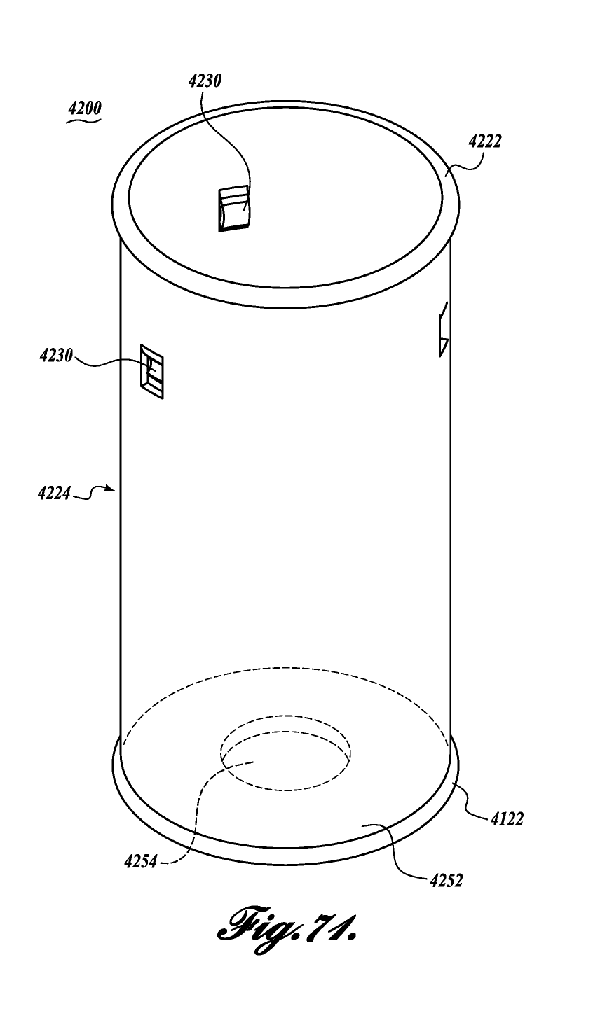

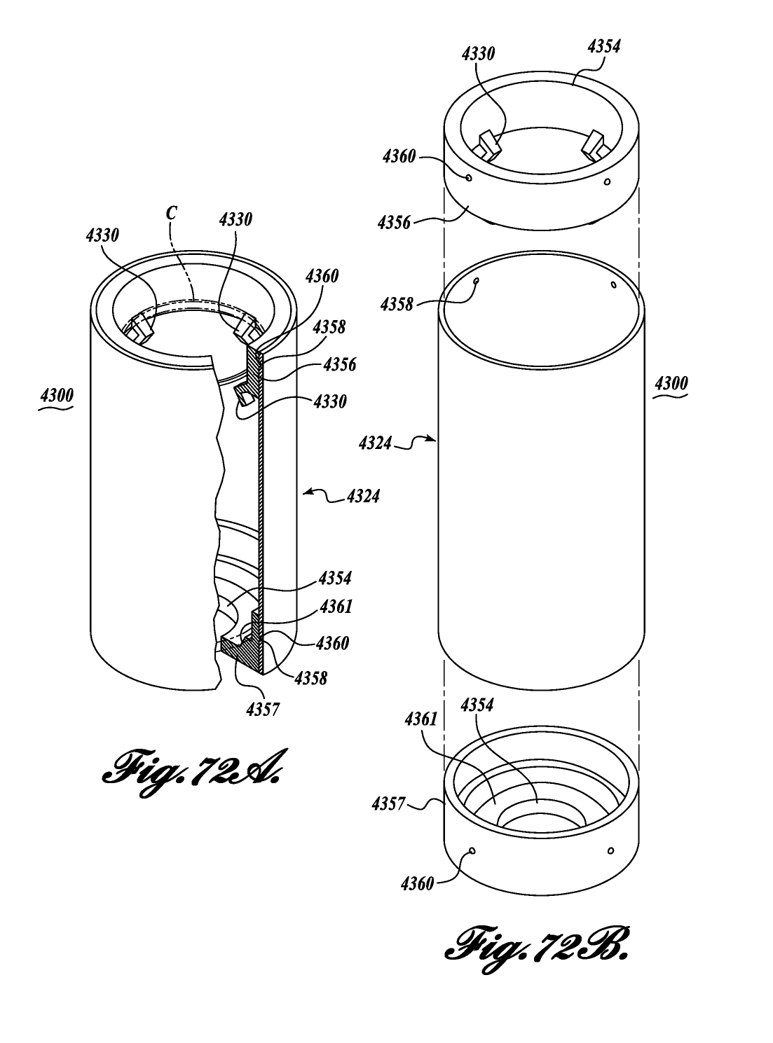

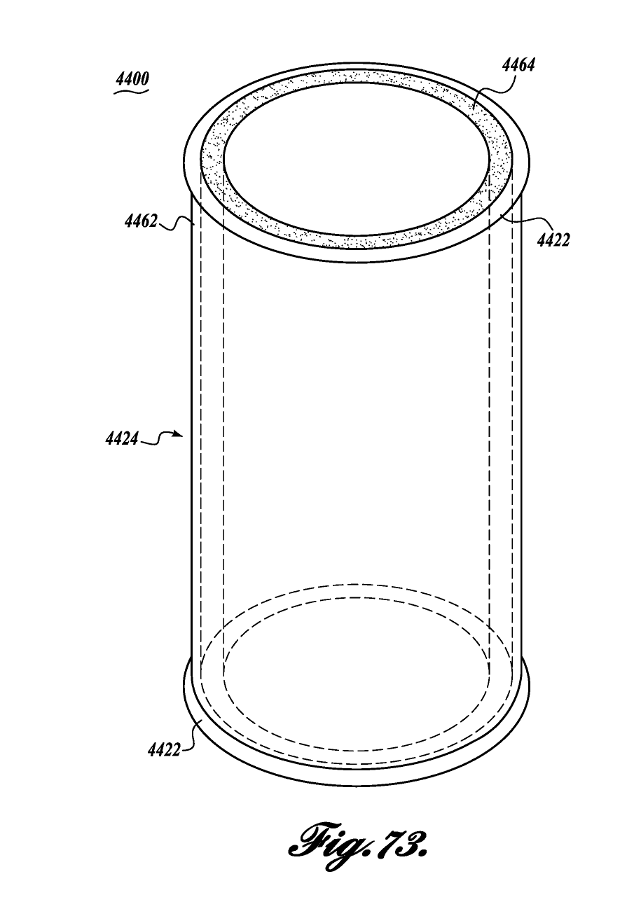

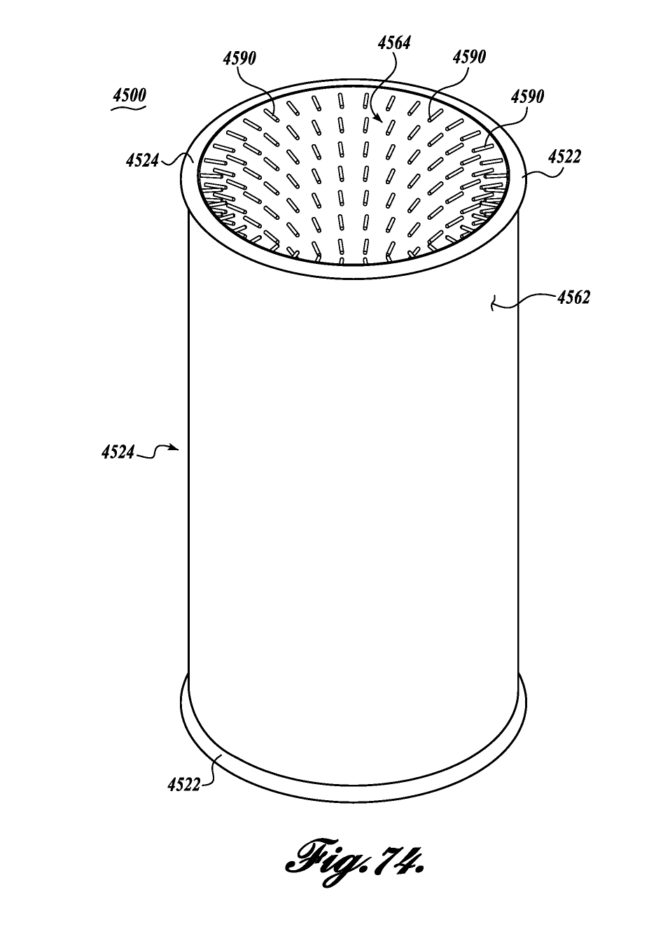

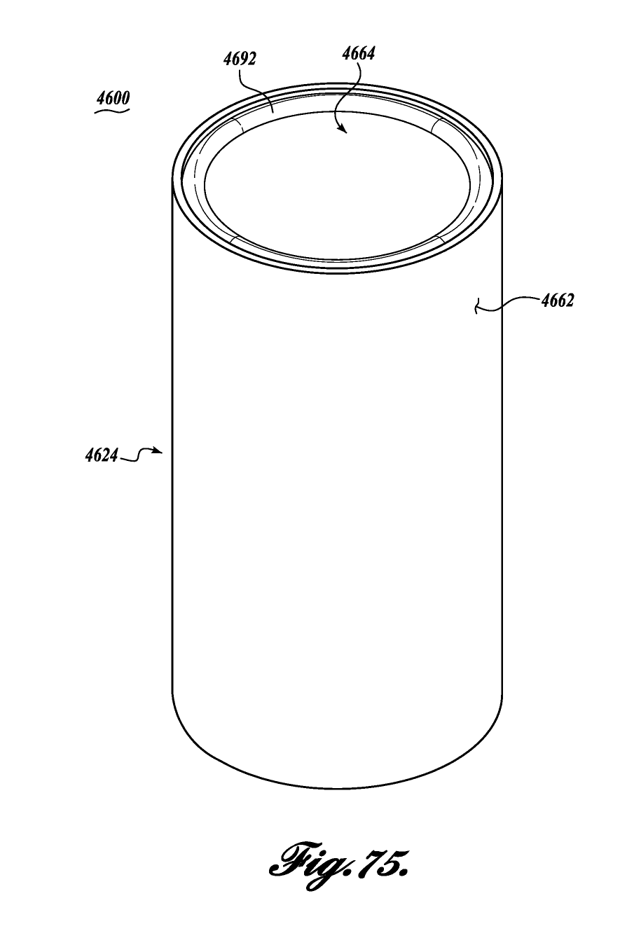

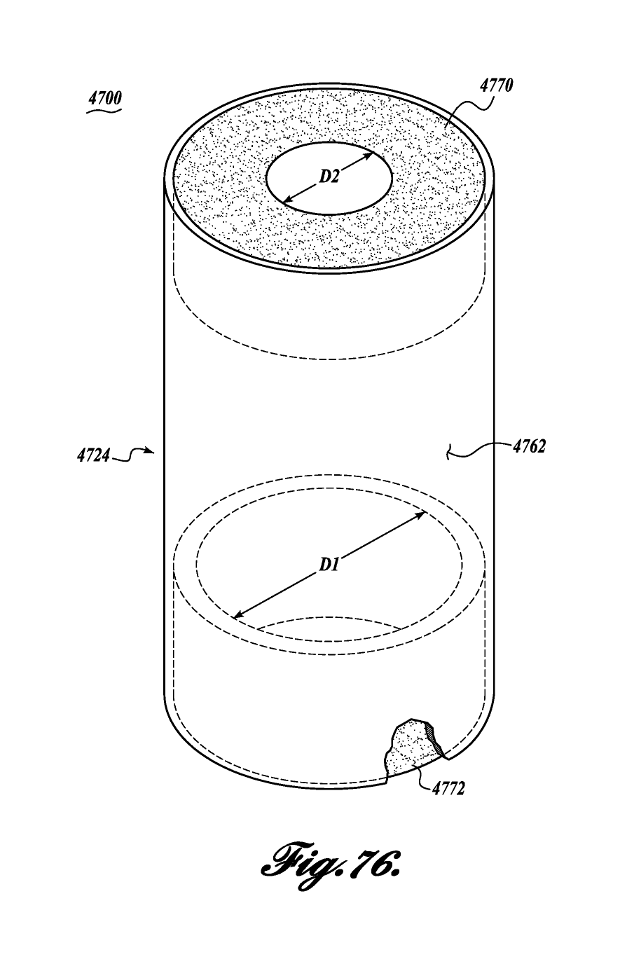

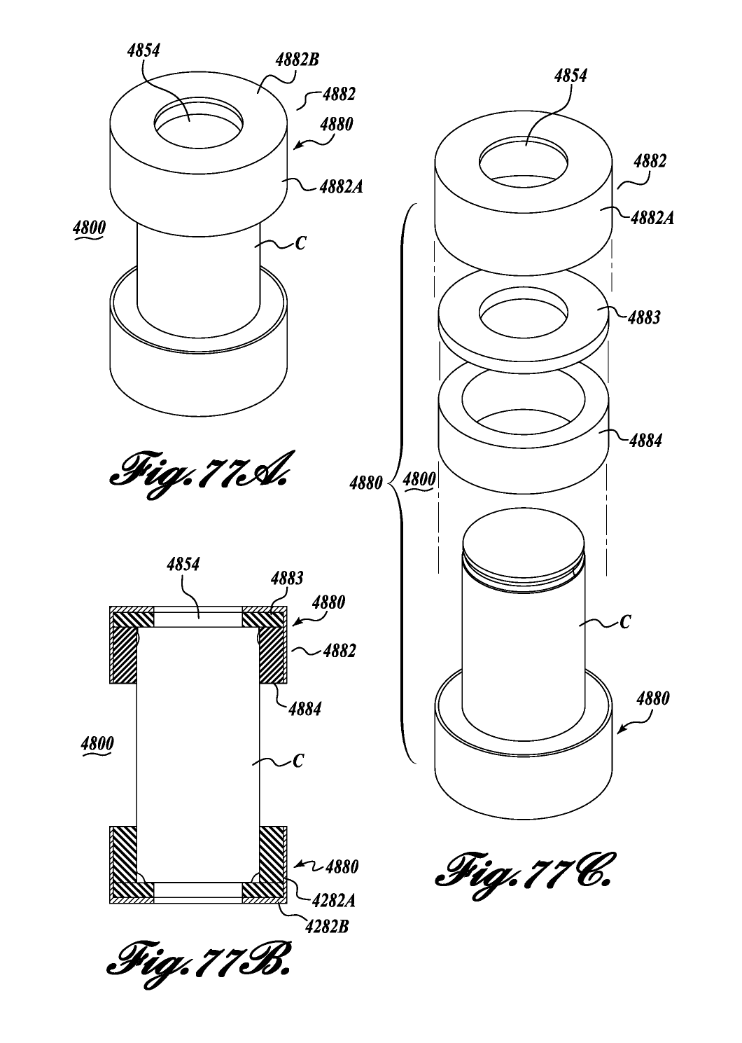

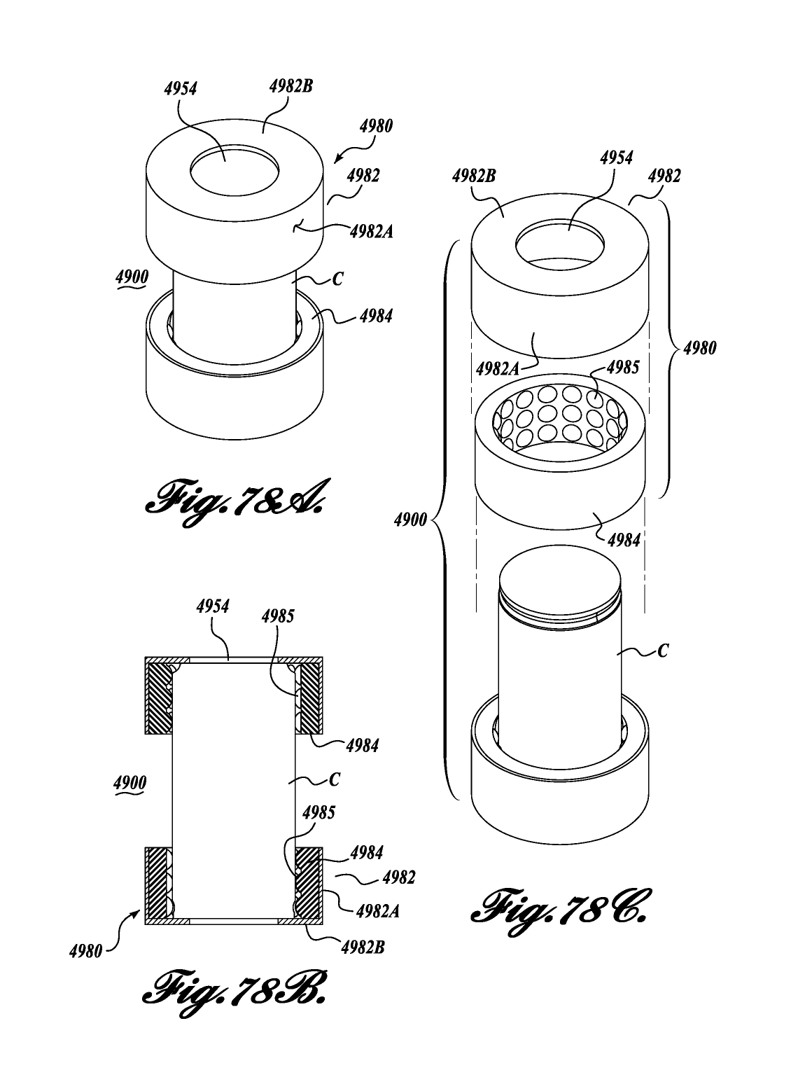

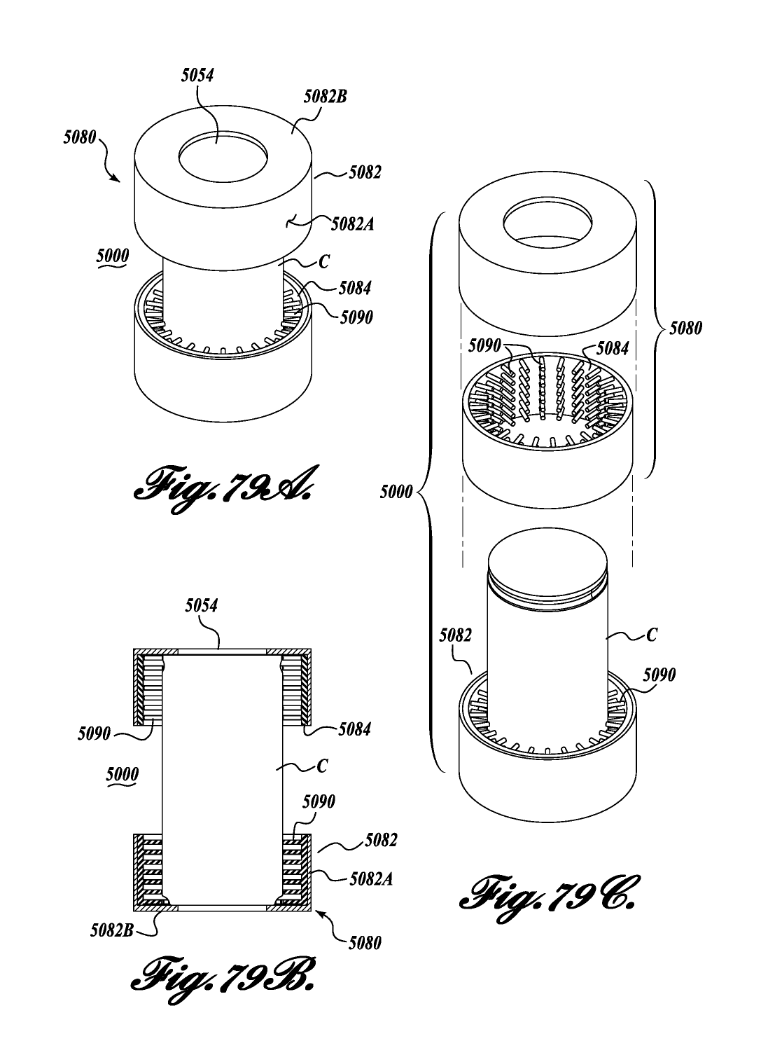

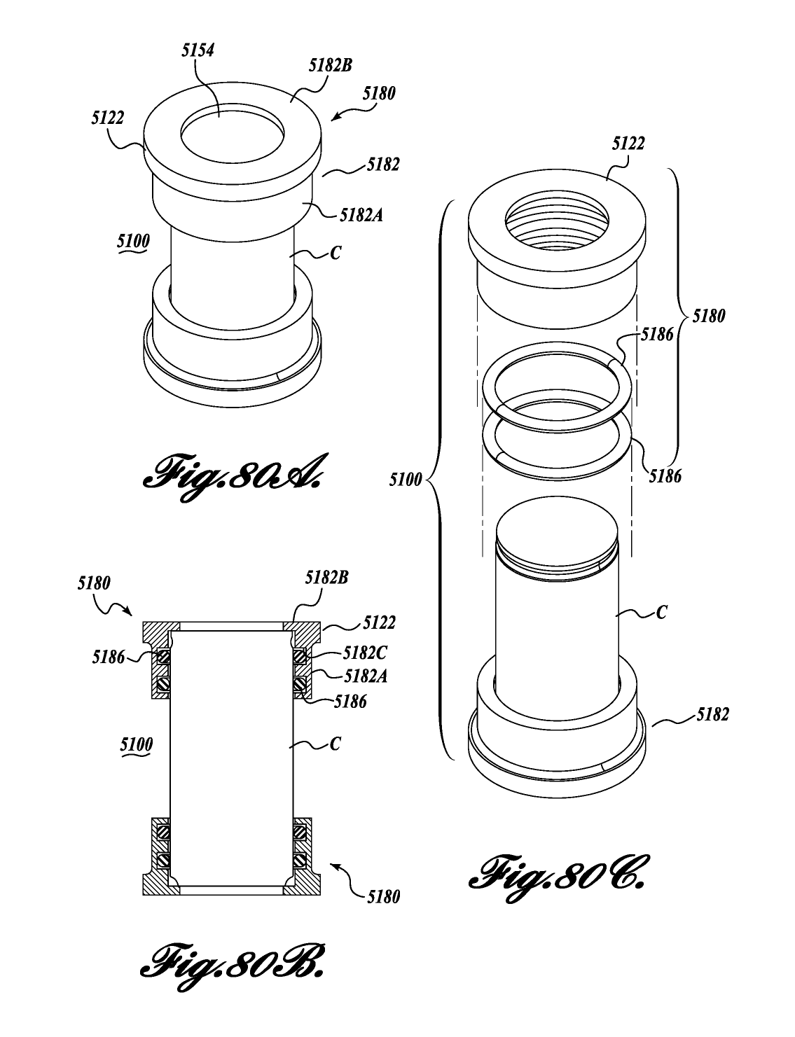

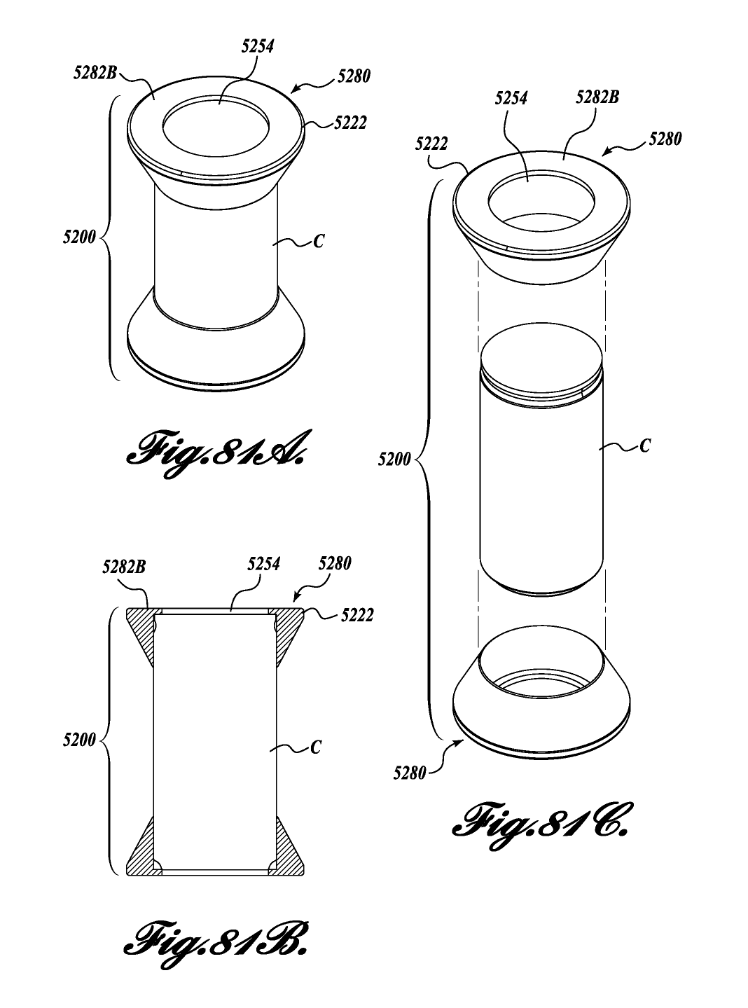

Now referring to FIGS. 30-38, various carrier embodiments are shown for carrying fragile or other irregularly-shaped containers, such as thin-walled cans, shaped cans, and shaped plastic bottles. Thin-walled cans are particularly fragile containers that can be damaged during continuous processes. The advantages of such carriers are as follows: (1) to prevent damage to the graphics on the cans; (2) to hold the can so that it will not be released from the carrier, and also to restrict movement to prevent rubbing; (3) to maximize the heat penetration through the carrier so that it reaches the contents inside the can; and (4) to handle irregular-shaped containers as though they are cylinders.

Referring to FIGS. 30-32, a carrier 1900 in accordance with one embodiment of the present disclosure is shown. The carrier 1900 is designed from molded plastic, such as polypropylene or peek. The carrier 1900 may be made from any suitable process, for example, injection molding process. Holes along the exterior surface of the carrier 1900 may be used to meeting molding parameters or reduce material usage, as well as provide access for processing fluids.

The design generally includes a shaped area 1910 in the bottom of the carrier 1900 for mating with the contoured bottom end of a typical two-piece can, and plastic tabs 1920 at the upper end for mating with the seam chines or other geometry on the top end of a typical can. The points of contact are therefore on the top and bottom ends of the can, to prevent rubbing, denting, or other damage to the seam chines and the outer cylindrical wall of the can. This carrier 1900 can also be used with a two-piece can, can loaded top-end first; with three-piece cans having either end being captured by plastic tabs; or with plastic bottles having tabs designed to capture an appropriate feature on the bottle.

Referring to FIGS. 33-35, a carrier 2000 in accordance with another embodiment of the present disclosure is shown. The carrier 2000 includes a main receiving portion 2002 and an insert portion 2004 for mating with the main receiving portion 2002. The insert portion 2004 may be formed from a compliant material, such as rubber or silicon. Like the carrier 1900 shown in FIGS. 30-32, the design includes a shaped area 2010 in the bottom of the carrier 2000 for mating with the contoured bottom end of a typical two-piece can. After the can has been received in the carrier 2000, the insert portion 2004 engages with the main receiving portion 2002 to hold the can in the carrier 2000.

Referring to FIGS. 36-38, a carrier 2100 in accordance with another embodiment of the present disclosure is shown. The carrier 2100 includes a main receiving portion 2102 and an insert portion 2104 for mating with the main receiving portion 2102. The insert portion 2104 may be formed from rubber or silicon or another suitable material having compressibility and flexibility properties. The bottom or receiving portion 2102 incorporates tabs 2106 that capture a feature on the can, such as the seam chines, to retain the can in the carrier 2100. An appropriate feature on a container is a groove that the tabs 2106 can fit into to restrict the container from falling out and to support the fragile surfaces. For cans, this feature may be the necked-in diameter adjacent the seam chines. For a plastic bottle, this feature may be the tapered upper shoulder that approaches the bottle neck. The tabs 2106 are preferably flexible enough to deflect when the container is inserted into the carrier 2100, but then to flex back into position when the container is removed from the carrier 2100.

Referring initially to FIGS. 39-43, a holder or carrier 2200 is illustrated as including end rims 2222 positioned at the ends of an elongate cylindrical body 2224 constructed as a compression sleeve. The body or sleeve 2224 has an outer diameter that is smaller than the diameter of the rims 2222. As shown in the figures, the body 2224 is of substantially constant diameter along its length except the end connection sections 2225 of the body flare or curve outwardly to join the larger diameter rims 2222. Ten longitudinal slots 2226 are formed in the body 2224 to extend the entire length of the cylindrical portion of the body and extend into the outwardly flared end connection portions 2225 of the body. The ten slots 2226 result in the formation of ten body strips or staves 2228, which press against a container C when the container is inserted into the carrier 2200, see, for example, FIGS. 42 and 43. The slots 2226 are shown as being somewhat narrower than the width of the staves 2228. However, as described in the other embodiments of the present disclosure, the relative widths of the slots and staves can be significantly varied, for example, to achieve different loadings on the container C. While the container C is shown as being in the form of a necked-in can, the container can be of other types of cans, bottles, or other types of containers.

As shown in FIG. 42, the container C is inserted into the carrier 2200 by simply pressing or pushing the can into the carrier. The carrier 2200 typically may be of a length longer than the height of a container C. In this regard, it may be desirable that the body section 2224 of the carrier itself be longer than the length of the container C.

It will be appreciated that by constructing the carrier 2200 with appropriate material, exhibiting sufficient flexibility, the staves 2228 of the body 2224 are capable of pressing against the can's exterior surface to hold the can securely in place within the carrier. To this end, the nominal interior diameter of the body section 2224 carrier is preferably smaller than the outer diameter of the container C to be held. In this regard, the carrier can be constructed from materials such as PEEK and PVDF.

It also will be appreciated that the overall size of the carrier, including the diameter of the rims 2222, is selected so as to be compatible with the rotary sterilizer to be utilized, and also to accommodate cans and bottles of a desired diameter and length. By utilizing the carrier 2200, containers C are held securely in place while being processed in a rotary sterilizer. During the processing, the containers do not contact the surfaces of the rotary sterilizer, and thus the containers are protected from scraping, scratches, or otherwise being damaged.

The carrier 2200 can be designed to apply a substantially uniform load or force on the container C along substantially the entire length of the staves 2228. This can be accomplished in various manners. For example, the thickness of the stave can be varied along its length. Specifically, the staves can be of thicker construction along the cylindrical portion of the carrier than at the location 2225 that the staves intersect the rims. This increases the rigidity of the staves in the thickened sections so as to enable the staves to apply a substantially uniform load against a can along the thickened portion of the staves. In this regard, see for example, FIGS. 46, 47, 48, 49, 50 and 51 and the corresponding descriptions for the embodiments of these figures below.

Alternatively, ridges can be formed along the interior of the staves to press against the can body. Such ridges can stand out from the interior of the staves a greater distance toward the longitudinal center of the carrier body 2224. As such, a substantially uniform pressure is imposed on the can substantially the entire length of the staves. Also, the ridges allow more access of the thermal process fluid to the can surface. In this regard, see FIGS. 59 and 60 and the corresponding descriptions of the embodiments of the carriers shown in the figures set forth below.

While the amount of the load applied to the cans can vary, it is desirable that the force applied to the can by the body be sufficient to substantially prevent the can from becoming unloaded from any staves during the sterilization process, including during the rolling of the carrier along the exterior shell of the rotary sterilizer and during transfer of the cans onto and out of the sterilizer. Thus, the required load applied to the can will vary with respect to the size of the can, including the diameter and length of the can.

For ease of loading and unloading, it is desirable that the carriers 2200 be symmetrical end-to-end. However, it is possible to construct a carrier so as to prevent a can from disengaging from at least one end of the carrier. In this regard, see for example FIGS. 46-50 illustrating a carrier 2500 constructed with tangs or tabs 2534 that project inwardly from rim 2522 to bear against the adjacent end of a container C. With respect to carrier 2500, the container C is inserted within the carrier from the opposite end. Thus, the can must be removed by pushing against the end of the can adjacent the tabs 2534. Also, projections or bumps 2530 can be located on the staves at the opposite end of the current carrier body 2524 to extend inwardly into the open center of the body. These projections or bumps can help prevent the container C from sliding out of the carrier 2500.

As discussed above, container C can be inserted into and removed from carrier 2200 by simply pressing the cans into the carriers, it is also possible to design the carriers so that an axial load may be applied against the underside of the rims 2222, thereby to pull the rims away from each other, longitudinally of the carrier 2200, which in turn will cause the staves 2228 to straighten relative to their attachment locations to the rims, and thus increase the internal diameter of the carrier so that the container can be easily slid into or out of the carrier. This method of loading and unloading the carrier may be needed if the sliding of the can into the carrier would cause the staves to scratch or otherwise damage the finish, including lithography, applied to the can exterior.

FIG. 44 illustrates a carrier 2300 that is similar to carrier 2200, except carrier 2300 includes eight staves 2328, and thus eight slots 2326. The components of carrier 2300 that are the same or similar to the components of carrier 2200 are given the same relative part numbers, but as a 2300 series number. This same numbering convention applies to the additional embodiments of the present disclosure shown in the remaining figures and as described below. Such components will not be repeated so as not to be unduly repetitive in describing the various embodiments of the carriers of the present disclosure. In FIG. 44, the slots 2326 are somewhat narrower in width than the staves 2328. Depending on the width of the slots 2326, the loading applied to container C by carrier 2300 can be greater or less than the loading applied to container C by carrier 2200.

FIG. 45 depicts carrier 2400, which is similar in construction to carrier 2300 shown in FIG. 46 except that carrier 2400 includes nine staves 2428, thus nine slots 2426. Moreover, the slots 2426 extend further endwise relative to the carrier 2400 so as to actually intersect with the inside diameters of the rims 2422. In addition, projections or bumps 2430 extend inwardly from the inside surfaces of selected staves 2428. These projections serve as abutments to stop the longitudinal movement of containers C held by the carrier 2400.

A further embodiment to the present disclosure is illustrated in FIGS. 46-50. The carrier 2500 illustrated in these figures is similar in construction to the carrier 2200 shown in FIGS. 39-43 with the exception that carrier 2500 includes nine slots 526 and thus nine staves 2528 formed about the perimeter of the body/sleeve 2524. Carrier 2200 shown in FIGS. 39-43 includes 10 slots 2226, and thus 10 staves 2228. The components of carrier 2500 shown in FIGS. 46-50 that are the same or similar to the components of carrier 2200 are given the same relative part number but as a 2500 series number.

In carrier 2500 the staves 2528 are thickened in their exterior along sections 2532, thereby adding rigidity to the staves. The thickened portions 2532 of the staves extend along the cylindrical portion of the carrier body 2524. The added thickness causes each of the staves in cross-section to be generally rectangular in shape with the apex of the cross-section extending radially outwardly relative to the longitudinal center of the carrier 2500. The added material or thickness to the staves 2528 along exterior sections 2532 causes these portions of the staves to be more rigid than without such increased thickness. As a consequence, a relatively large and relatively uniform load can be applied to the exterior of a container being held.

Moreover, projections or bumps 2530 extend inwardly from the end portions of selected staves 2528. As noted above, such projections can serve to keep the container from sliding out of the carrier during processing. In addition, the carrier 2500 includes tabs 2534 that extend radially inwardly from the ends of the staves 2528 opposite to the location of the projections 2530. The tabs 2534 extend diagonally from the staves and then curve or otherwise extend transversely inwardly relative to the length of the staves so as to form shoulders or abutments to bear against the end portions of cans carried by the carrier 2200.

A further embodiment of the present disclosure is illustrated in FIG. 51 wherein the carrier 2600 is similar in construction to the carrier 2400 shown in FIG. 49 in that nine staves 2628, and thus nine slots 2626, are utilized. However, unlike carrier 2400, and in the manner of carrier 2500, the exterior of the staves 2628 are thickened along exterior sections 2632. Such exterior sections also are, for example, triangular in cross-section in a manner similar to the thickened portions 2532 of staves 2528. Of course, other shapes having a given or varying thickness may be utilized to increase rigidity to the carrier sections. A container C is shown as being held by the carrier 2600 in FIG. 51.

FIGS. 52 and 53 depict carriers 2700 and 2800 that are similar in construction to each other. Carriers 2700 and 2800 differ from the carriers described above in that such carriers utilize six staves 2728 and 2828 that are relatively wider than the slots 2726 and 2826 that separate the staves from each other. One difference between carrier 2800 versus carrier 2700 is that shallow grooves 2823 extend around the circumference of the rims 2822 of the carrier 2800. Depending on the type of material used and the thickness of the material used to form the staves 2728 and 2828, the carriers 2700 and 2800 can apply a larger holding load against container C than if a larger number of grooves were to be used or if the grooves 2726 and 2826 were wider relative to the staves 2728 and 2828. As noted above, the carriers 2700 and 2800, as well as the other carriers disclosed herein, can be composed of a range of materials, including PEEK and PVDF.

FIG. 54 depicts a carrier 2900 that is similar in construction to carrier 2700 shown in FIG. 52 with the exception that circular enlargements 2927 are formed at the ends of the slots 2926. This somewhat reduces the width of the staves 2928 at the location of the curved end portions 2925 of the staves 2928 which intersect the rims 2922.

FIG. 55 depicts a carrier 3000 that is similar in construction to carriers 2700 and 2800 in that the staves 2928 are relatively wider than the corresponding grooves 2926. However, carrier 3000 utilizes eight staves rather than six staves utilized in carriers 2700 and 2800. Another difference between carrier 3000 and carriers 2700 and 2800 is that the slots 2926 extend further along the carrier body 2924 than in carriers 2700 and 2800.

FIG. 56 depicts carrier 3100 that is similar in construction to carrier 2600 shown in FIG. 51 but with enlargements 3127, generally circular in shape, formed at the ends of the slots 3126. The enlargements 3127 reduce the width of the staves 3128 at the curved intersection 3025 of the staves and the rims 3122.

FIG. 57 depicts a carrier 3200 that is similar in construction to carrier 2500 shown in FIGS. 48, 49, and 50 with the exception that projections 3230 are formed at the end portions of each of the staves 3228. Moreover, circular enlargements 3227 are formed at the end of each of the slots 3226 in a manner similar to that employed with carrier 3100 shown in FIG. 56. As in carrier 2500 shown in FIGS. 48, 49 and 50, tabs 3234 extend inwardly from selected staves 3228 at the ends of the staves opposite the projections 3230.

FIG. 58 depicts carrier 3300 which is similar in construction to carrier 3100 shown in FIG. 56, but with diamond-shaped enlargements 3327 formed at the end of slots 3326. These diamond-shaped enlargements significantly narrow the width of the staves 3328 at the location 1325 that the staves intersect rim portions 3322. As in carrier 3100, the staves 3328 have thickened exterior sections 3332 extending along the exterior sides 3332 of the staves 3328.

FIGS. 59, 60, 61, and 62 depict carriers 3400, 3500, 3600, and 3700 that each utilize six slots 3426, 3526, 3626, and 3726, respectively. As a consequence, each of the carriers 3400, 3500, 3600, and 3700 also utilize six staves 3428, 3528, 3628, and 3728, respectively. Moreover, the exterior of each of the staves is thickened at sections 3432, 3532, 3632, and 3732. Rather than causing the staves to be triangular in cross-section due to the increased exterior thickness as in carriers 2500 and 2600, above, the thickened sections of the staves of carriers 3400, 3500, 3600, and 3700 in cross-section resemble a truncated triangle. Moreover, rather than being of substantially uniform thickness as shown in stave sections 3432, 3532, and 3632, the thickened stave section 3732 of carrier 3700 is actually thinned at about the longitudinal midpoint of the staves 3728. This provides the midpoint of the staves with somewhat more flexibility than the remainder of the stave.

Also in carriers 3400 and 3500, the staves 3428 and 3528 are thickened in the inward side of the staves, thereby to define inwardly created ribs 3433 and 3533, respectively.

In each of the carriers 3400, 3500, 3600, and 3700, circular enlargements 3437, 3537, 3637, and 3737 are formed at the ends of the respective slots 3426, 3526, 3626, and 3726. In carriers 3400 and 3500, the ends of slots 3426 and 3526 flare out to tangentially intersect with the enlargements 3437 and 3537. Further, in carriers 3600 and 3700, additional slots 3640 and 3740 are formed at the intersections 3625 and 3725 of the staves 3628 and 3728 with corresponding rim portions 3622 and 3722. These slots 3640 and 3740 result in the ends of the staves 3628 and 3728 intersecting the rim portions 3622 and 3722 via relatively narrow strips 3644 and 3744. The purpose of such slots is to control the stresses introduced into strips 3644, 3744, and thus also the stress in the connector sections 3625 and 3725 during loading and unloading of container C. In carrier 3600, the slots 3640 are generally in the shape of an elongated triangle, with the apex of the triangle adjacent the rim 3622 and the base of the triangle located toward the stave 3628. The slots 3740 of carrier 3700 are similar in shape to the slots 3640 but extend a further distance along the stave 3728 than in the carrier 3600.

The carrier 3500 further differs from carriers 3400, 3600 and 3700 in that pairs of projections 3530 are formed at the ends of the interiors of the staves 3528. In addition, shallow longitudinal grooves 3542 extend along the interior of the staves 3528. These grooves can assist in reducing a potential “hydroplaning” type effect between the interior surface of the sleeves and the exterior of container C, causing container C to slip out of the carrier 3500.

FIG. 63 depicts a carrier 3800 that is similar in construction to carrier 3700 shown in FIG. 62, with the exception that in FIG. 63, the slots 3840 at the end portion of the staves 3828 are triangular in shape rather than an elongate shape in the form of the slots 3740 shown in FIG. 62. The slots 3840 are oriented so that the base of the triangular shape extends adjacent the inside circumference of the rim 3822, and the apex of the slot extends toward the longitudinal center of the stave 3828. The two sides of the triangular slot 3840 are curved to match the curvature of the adjacent portions of the longitudinal slots 3826 of the carrier 3800. The exterior of the staves 3828 are increased in thickness at locations 3832 in the manner of thickened location 3732 shown in FIG. 62.

A further embodiment of the present disclosure is shown in FIGS. 64, 65, 66, and 67 wherein the depicted carrier 3900 resembles aspects of carrier 3800 shown in FIG. 63 and carrier 3500 shown in FIG. 60. As in carrier 3800, the carrier 3900 includes six staves 3928 that are separated by longitudinal slots 3926, but with the staves 3928 perhaps somewhat wider than the staves 3828. Triangular openings 3940 are formed at the end sections 3925 of the carrier at the location that the staves 3928 intersect the rims 3922. Also, in carrier 3900, the ends of the longitudinal slots 3926 are not quite as rounded as the slots 3826 shown in FIG. 63.

In a manner similar to carrier 3500, the carrier 3900 includes a plurality of projections 3930 formed at the ends of the staves 3928 at the inside surfaces thereof. Also in a manner similar to holder carrier 3500, shallow grooves 3942 extend longitudinally along the inside surface of the staves 3928. Also similarly to carrier 3500, the exterior of the staves 3928 in carrier 3900 are thickened relatively uniformly in the outward direction of the staves, thereby adding rigidity to the longitudinal sections of the staves.

FIG. 65 shows a container C held by the holder 3900. FIG. 66 is a cross-sectional view of container C showing the container C held in place. Also shown is the thickness profile of the staves 3928. FIG. 67 depicts a container C held in place within holder 3900.

FIGS. 68 and 69 depict a further embodiment of the present disclosure, wherein a carrier 4000 is illustrated as having rim portions 4022 that extend a further distance along the longitudinal direction of the carrier relative to most of the carriers described and illustrated above. Also, carrier 4000 uses four relatively wide staves 4028, separated by relatively narrow slots 4026. Also as shown in FIGS. 68 and 69, the staves 4028 extend along the interior of the rim portions 4022. Further, the staves 4028 are tapered or beveled in the outward direction along their lengths within the carrier rims 4022. This taper creates a “lead in” for container C when the container is placed within the carrier 4000. Moreover, the interior of the staves 4028 can include inwardly directed ridges or rims that bear against the exterior of container C. In addition, rectangularly shaped slots 4040 are formed at the intersection of the staves 4028 and rim 4022. The slots 4040 are positioned laterally, substantially in the middle of the width of the staves 4028. This construction can assist in reducing the resistance to the sliding of the container C within the interior of the carrier 4000 when the container C is being introduced into the carrier. As can be appreciated, the carrier 4000 is of rugged construction, while still permitting the container C to be conveniently loaded into the carrier and removed from the carrier without damage to the lithographed exterior of the container.

FIG. 70 depicts a further embodiment of the present disclosure, wherein a carrier 4100 is illustrated as being substantially cylindrical in shape, and having open end portions. Chines 4122 can be formed at the ends of a cylindrical body portion 4124, or the body portion can be formed without chines. The body can be formed from an elastic or resilient or compressible material for receiving a container, such as container C, and then holding the container within the carrier during processing of the container contents.

Features such as curved protrusions or ramps 4130 can be formed at the end portions of the body 4124 to hold the container C within the carrier 4100, see FIG. 70B. The protrusion/ramp can be created by making a generally U-shaped cut though the container and then forming the cut portion of the can into a curved protrusion that extends into the interior of the body 4124. As a result a slot 4131 is created in the body 4124 from the U-shaped cut. The protrusions or ramps are constructed to flex outward into and perhaps at least partially beyond the slot 4131 when a container is inserted into or removed from the carrier 4100. When a container is inserted into or removed from the carrier 4100, the protrusions/ramps slide against the surface of the container, causing the protrusions/ramps to flex outwardly. Once the can passes the protrusion/ramp, the protrusion/ramp can assume its original position as shown in FIG. 70B. It will be appreciated that the curved protrusions/ramps serve to retain the container C within the carrier 4100.

Although not shown, slots can be formed in the body 4024 to facilitate heat transfer between the medium in the rotary sterilizer and the contents of the container C. Such slots can take many different forms. For example, the slots may extend along the length of the body in a manner similar to, for example, slots 2226, shown in FIGS. 39-43. Rather than using slots, round, rectangular, or other shaped openings or holes may be formed in the body 4124 for the same thermotransfer purpose.

FIG. 71 depicts a further embodiment of the present disclosure wherein the carrier 4200 is illustrated as being constructed similarly to carrier 4100, with the exception that one end of the body 4224 is closed off by an end wall 4252. A central hole 4254 is formed in the end wall 4252, which may be used to enable a plunger or other type of device to eject a container C from the holder 4200. Of course, other/additional holes may be formed in the end wall 4252 to facilitate heat transfer to or from the contents of the container C. Also, as in carrier 4100, protrusions or ramps, or other features 4230 can be formed in the body 4224 to retain the container C within the carrier 4200. The carrier 4200 can be constructed from one or more materials, either the same or similar to the materials used to construct carrier 4100.

FIG. 72 depicts a further embodiment of the present disclosure, wherein carrier 4300 is illustrated as having a body portion 4324 in the form of a sleeve for receiving a container C therein, as well as end plugs 4356 and 4357 for capturing the container within the body 4324. The end plug 4357 is sized to fit snugly within one end of the body portion 4324. In this regard, the end plug 4357 includes a cylindrical wall section as well as a transverse end section. The transverse end section is formed with shoulder 4361 for receiving one end of container C. The shoulder 4361 is curved or countered to match the shape or contour of the received end of the container C. A centered opening 4354 is formed in the end plug 4357 to facilitate heat transfer with the contents of container C. It will be appreciated that the end plug 4357 can be constructed of various configurations so as to receive containers C of different shapes, contours and sizes while the diameter of the body 4324 can remain the same.

With respect to end plug 4356, such end plug also is snugly receivable within the body 4324 opposite to the location of the end plug 4357. The end plug 4356 includes a cylindrical section that closely fits within the inside diameter of the body 4324. A plurality of formed tabs 4330 project from the cylindrical section into the body 4324. The tabs are curved or rounded to present a generally convex protrusion or detent toward the interior of the body 4324. The tabs 4330 are configured to flex outwardly when a container, such as container C, is either inserted into the carrier by simply pressing the container into the sleeve 4324 or when removing the container C by pushing the container outwardly past the interior of the plug 4356. In this manner, the tabs 4330 operate in a manner similar to other tabs described above, including tabs 4230, 4130, and 1920 described above to retain the container C within the carrier 4300. As will be appreciated, the center of the plug 4356 is substantially open to allow circulation of heating fluid through the carrier 4300.

It also will be appreciated that, as with end plug 4357, the end plug 4356 can be replaced with an end plug of a different configuration so as to accommodate containers C of different sizes and shapes, including different lengths and different diameters, while utilizing the same exterior sleeve 4324, thereby resulting in economies of manufacture and also reducing the number of container sleeves needed while still being able to process containers of different shapes and sizes.

Openings or holes can be formed in the body portion 4324 to facilitate heat transfer with the contents of the container C. The body 4324 can be formed from a durable material, such as a high strength plastic or metal. The end plugs 4356 and 4357 can be constructed from a softer, more flexible, material such as a high strength plastic or metal. Also, to retain the end plugs 4356 and 4357 within the body 4324, indentations 4358 can be formed in the exterior of the body 4324 and corresponding protrusions, bumps, or similar features 4360 can be formed to project outwardly from the cylindrical sections of the end plugs to mate with the indentations 4358. Of course, numerous systems can be used to retain the plugs 4356 and 4357 within the body 4324. For example, a shallow groove may be formed around the interior of the end plugs 4356 and 4357, and corresponding ridges, series of bumps, or other protrusions may be formed on the exterior of the body 4324 to engage with the groove formed in the plugs 4356 and 4357.

As with carriers 4100 and 4200, slots can be formed in the body 4324 to facilitate heat transfer with the contents of the container C. Also, rather than utilizing slots, holes, perforations, or other through openings can be formed in the body 4324 to facilitate heat transfer with the contents of the container C.

FIG. 73 depicts a further embodiment of the present disclosure wherein a carrier 4400 is illustrated as having a body 4424 composed of an outer shell 4462 and an inner liner 4464. The outer shell can be composed of a durable, high-strength material, such as a metal or high strength plastic. The inner liner 4464 can be composed of a resilient, deformable, compressible material, for example, a softer plastic material, a foam material, etc. The outer cylinder portion 4462 can include chines 4422 at its ends to facilitate the rolling of the carrier 4400 within the rotary sterilizer, as described above. As in the other carriers described above, slots or other openings can be formed in the body portion 4424 for facilitating heat transfer with the contents of the container C held by the carrier 4400.

FIG. 74 depicts a further embodiment of the present disclosure, wherein a carrier 4500 is shown as having a body 4524 composed of an outer shell 4562 and an inner liner 4564. As in the outer shell 4462 of carrier 4400, the outer shell 4562 can be composed of a durable, high-strength material such as a metal or high-strength plastic. Further, the inner liner 4564, such as inner liner 4464 shown in FIG. 73, can be composed of a resilient, deformable, compressible material, for example, bristles 4590 that extend inwardly toward the longitudinal center of the carrier 4500. The bristles can be of sufficient stiffness and quantity and length to securely hold the container C within the carrier 4500, but not damage the lithograph exterior of the container. Thus, bristles 4590 can be composed of either natural material or synthetic material, such as a plastic material.

As can be appreciated, the load placed on the container C by the bristles 4590 can be a function of the density or number of bristles as well as the length of the bristles, the stiffness of the bristles, etc. Moreover, the physical characteristics of the bristles can differ along the length of the interior of the liner 4464. For example, the bristles 4590 located near the ends of the inner liner 4564 can be of stiffer construction, of longer length and/or of greater density than in the remainder of the inner liner 4564. In this manner, the bristles 4590 at the ends of the inner liner 4564 can serve to help prevent longitudinal movement of a container C when disposed within the carrier 4500.

Also, as in carrier 4400, the outer cylindrical portion 4562 can include chines 4522 at its ends to facilitate the rolling of the carrier 4500 within the rotary sterilizer as described above. Also, as in the other carriers described above, slots or other openings can be formed in the body portion 4524 for facilitating the heat transfer to or from the contents of the container C held by the carrier 4500.

FIG. 75 depicts a further embodiment of the present disclosure wherein a carrier 4600 is illustrated as having a body 4624 composed of an outer shell 4662. As with outer shells 4462 and 4562, the outer shell 4662 can be composed of a durable, high-strength material, such as a metal or a high-strength plastic. Moreover, as in inner liners 4464 and 4564, the carrier 4600 includes an inner liner 4664. Such inner liner is illustrated as in the form of a cylindrically shaped bladder 4692 disposed within and attached to the outer shell 4662. The bladder 4692 may be filled with a gas, such as air, helium, or other gas. The bladder 4692 is sized so as to compress somewhat when receiving a container C within the carrier 4600. Moreover, when the container C is being processed within a rotary processor, the heat applied to the container C can cause the air or other gas within the bladder 4692 to expand, thereby resulting in the bladder imposing an increased compressive force against the exterior of the container C. As a result, the container C is securely held within the carrier 4600.

FIG. 76 depicts a further embodiment of the present disclosure, wherein carrier 4700 includes a body 4724 composed of an exterior cylinder 4762 and two spaced apart interior liner sections 4770 and 4772, disposed toward the end portions of the cylinder portion 4762. The container C is held by the liner sections 4770 and 4772, whereas the interior of the cylinder 4762, between the liner portions, is open for reception of heating fluid from the sterilizer. In this regard, slots or openings may be provided in the cylinder portion 4762 of the carrier 4700.

As shown in FIG. 76, liner section 4772 is substantially in the form of a circular ring or collar defining an interior diameter D1. On the other hand, the liner section 4770, though also generally in the shape of a ring or collar, has a substantially smaller interior diameter D2. The smaller diameter D2 is designed to receive the reduced size neck portion of a container C, whereas the larger diameter D1 is sized to accept the body portion of the container C. Of course, if the container C is of substantially constant diameter, then the liner section 4770 and 4772 can be sized accordingly. Moreover, the liner sections 4770 and 4772 can be composed of a foam material, for example, of the type used to form liner 4464 in FIG. 73, or bristles such as bristles 4590 used to form the liner 4564 in FIG. 74 or a bladder such as bladder 4692 used to form the inner liner 4664 shown in FIG. 75.

Next, FIGS. 77A, 77B, and 77C depict a further embodiment of the present disclosure, wherein carrier 4800 is illustrated as composed of two end cap assemblies 4880 sized to receive the ends of a container, such as container C. The exteriors of the end cap assemblies 4880 are cylindrical so as to roll within a rotary processor. The interiors of the end cap assemblies may also be cylindrical to match the exterior shape of the container C, or the interiors of the cap assemblies can be of other shapes to match the exterior shapes of the containers being carried. Holes 4854 may be formed in the ends of the end cap assemblies to facilitate removal of the end cap assemblies from the container and also to enhance heat transfer to the contents of the container C.

The end cap assemblies 4880 are formed from an outer housing, shell or skin 4882 of substantially high strength rugged material, such as metal or a high strength plastic. Each housing 4882 includes a cylindrical section 4882A and an end wall 4282B disposed across one end of the cylindrical section.

A circular end insert 4883 and a cylindrical liner insert 4884 are snugly disposed within the housing 4882 to securely receive the container C therein. The end insert 4883 and liner 4884 can be formed from a material that may be compressible, deformable, elastic, etc., so as to receive the container C, and then securely hold the container C in place. As with the other carriers disclosed above, such as in FIGS. 73, 74, and 75, as a non-limiting example, the liner insert 4884 can be composed of foam material, bristles, a bladder, etc. It will be appreciated that carrier 4800 composed of the two end cap assemblies 4880 protect the container C from damage during processing in a rotary processor.

Next, FIGS. 78A, 78B, and 78C depict a further embodiment of the present disclosure wherein carrier 4900 is illustrated as composed of two end cap assemblies 4980 composed of an exterior shell or housing 4982 that is shaped similarly to end cap housings 4882. In this regard, each of the end cap assemblies 4980 has a cylindrical section 4980A and an end wall 4980B with a central hole formed in the end wall. A deformable/compressible cylindrically shaped liner insert 4984 is sized to be closely receivable within the end cap housing 4982. The inside diameter of the insert 4984 is sized to snugly receive the can C. As shown in FIGS. 78A, 78B, and 78C, two end cap assemblies 4980 are utilized to protect and support the can C during processing with a rotational retort system. In this regard, the insert 4984 can be made from numerous materials, for instance, an elastomeric material such as a closed cell foam material. The insert 4984 can be sized to securely receive the container C and maintain engaged with the end of the container C during processing of the container. In this regard, the insert 4984 is constructed with a series of bumps 4985 that extend inwardly from the interior surface of the insert 4984 forward the longitudinal center of the carrier. Such bumps 4985 are compressed when receiving the container C. The bumps 4985 can facilitate securing the container C within the end cap assemblies and also allow for the unobstructed removal of the container C from the end cap assemblies, since the entire interior surface of the insert 4984 is not in direct contact with the exterior of the container C.

FIGS. 79A, 79B, and 79C illustrate a further embodiment of a carrier 5000 in accordance with the present disclosure. The carrier 5000 is similar in construction to the carriers 4800 and 4900, described above. Thus the components of carrier 5000 that correspond with carriers 4800 and 4900 are numbered similarly but within the 5000 series.

One difference between carrier 5000 and carriers 4800 and 4900 is that the interior of the insert 5084 of the end cap assemblies 5080 includes a plurality of inwardly extending fingers or bristles 5090. The bristles 5090 can be similar to bristles 4590, described above, or can be of a different construction consisting of a relatively fewer number of individual deformable fingers. Other than with the utilization of the bristles or fingers 5090, the construction of the end cap assemblies 5080 can be very similar to the construction of end cap assemblies 4880 and 4980, described above.

FIGS. 80A, 80B, and 80C illustrate a further embodiment of the present disclosure wherein a carrier 5100 is constructed from a pair of end cap assemblies 5180 having housings 5182 in the form of a cylindrical section 5182A and an end wall 5182B. A pair of interior grooves 5192C formed therein for receiving a pair of O-rings 5286. The inside diameters of O-rings 5286 are sized to snugly engage over the outside diameter of container C. The end cap assemblies 5180 can be a symmetric construction in cross-section, as shown in FIG. 80B, for containers of the same size and shape at each end. Or, one of the end cap assembly 5180 may have a smaller or different shaped inside diameter than the other end cap assembly, thereby to accommodate a container C that is different in size and/or shape at one end from the other end.

A further embodiment of the present disclosure is illustrated in FIGS. 81A, 81B, and 81C wherein a carrier 5200 is composed of a pair of formed end caps 5280 in the form of a compressible or deformable boot structure constructed from an elastomeric or other suitable material. Each formed end cap 5280 includes a rim portion 5222 and an end wall 5282B having a central hole 5254 formed therein. The inside diameter of each end cap 5280 is shown as cylindrical in shape and sized to snugly receive the end portion of container C therein. It will be appreciated that the interior of the end caps can be sized and shaped to correspond to the sizes and shapes of the container C being held by the carrier 5200.

From the rim portion 5322, the end caps 5380 taper inwardly to define a bevel. This reduced diameter thickness of the end caps 5380 enables the end caps to be expandable to engage over the outside diameter of container C, while still applying sufficient compressive force on the container to securely retain the container in engagement with the end caps 5280.

A further embodiment of the present disclosure is illustrated in FIGS. 82A and 82B, wherein a carrier 5300 is constructed similarly to other carriers, described above, including carriers 2200 and 3000. However, carrier 5300 is composed of two halves 5387 in a clamshell type manner for ease of receiving container C therein and for removal of container C from the carrier 5300. The two halves 5387 of the carrier 5300 can be hinged by any convenient means. For example, the material 5387A of the rim portions 5322 at the intersection of the two halves 5387 can be of reduced thickness to enable the two halves 5387 to be opened at the center section. Also the two halves can be secured in closed position by numerous means, including by a snap fit, for example, by a finger projecting (not shown) from one of the halves 5387 to engage with a notch (not shown) formed in the other half. Alternatively, or in addition, an expandable garter or band (not shown) can be simply placed over the outside diameter of the body portion 5324 or other portion of the carrier 5300. It will be appreciated that other than being constructed from two halves, the carrier 5300 can be very similar to other carriers, described above, including carriers 2200 and 3000.

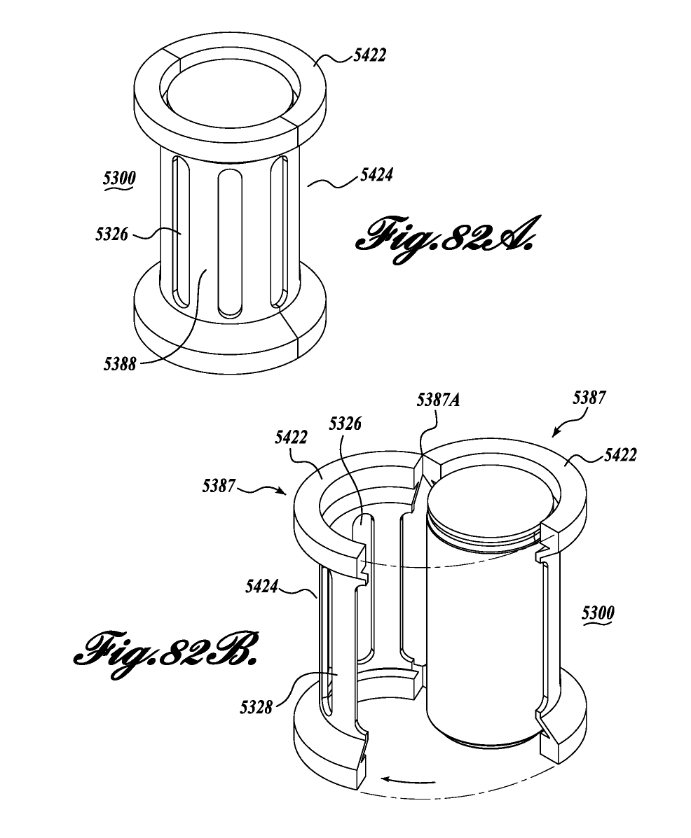

Referring to FIGS. 83A, 83B, and 83C, these depict a further carrier 5400 constructed from two halves 5487. The carrier 5400 is similar in construction to carrier 5300, except that the two carrier halves 5487 engage with each other differently. As shown in FIG. 82C, in each half 5481, the semi-circular rim portions 5422 either terminate in a key 5488A or a slot or keyway 5488B to receive the key 5488A of the opposite half 5487. As shown in FIG. 83C, the two carrier halves 5487 are assembled by conveniently sliding the two halves longitudinally into engagement with each other so that corresponding keys 5488A engage with corresponding keyways 5488B. Appropriate means can be provided for maintaining the two halves in engagement with each other. When assembled, the carrier 5400 is similar to carriers described above, including carriers 2200 and 3000.

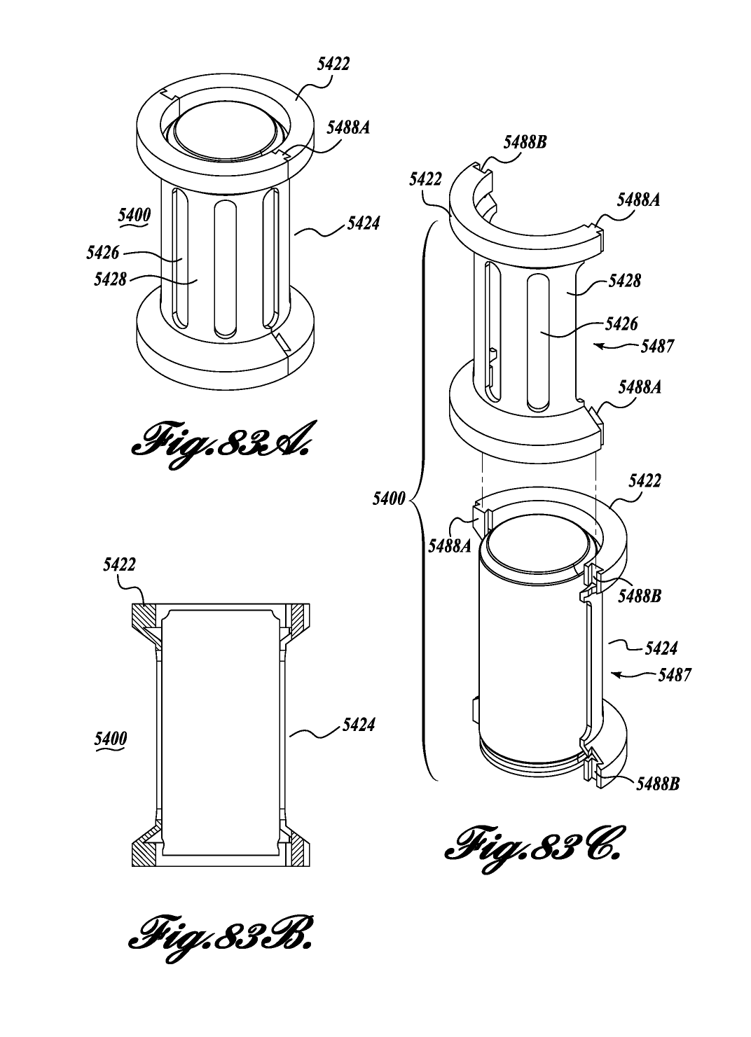

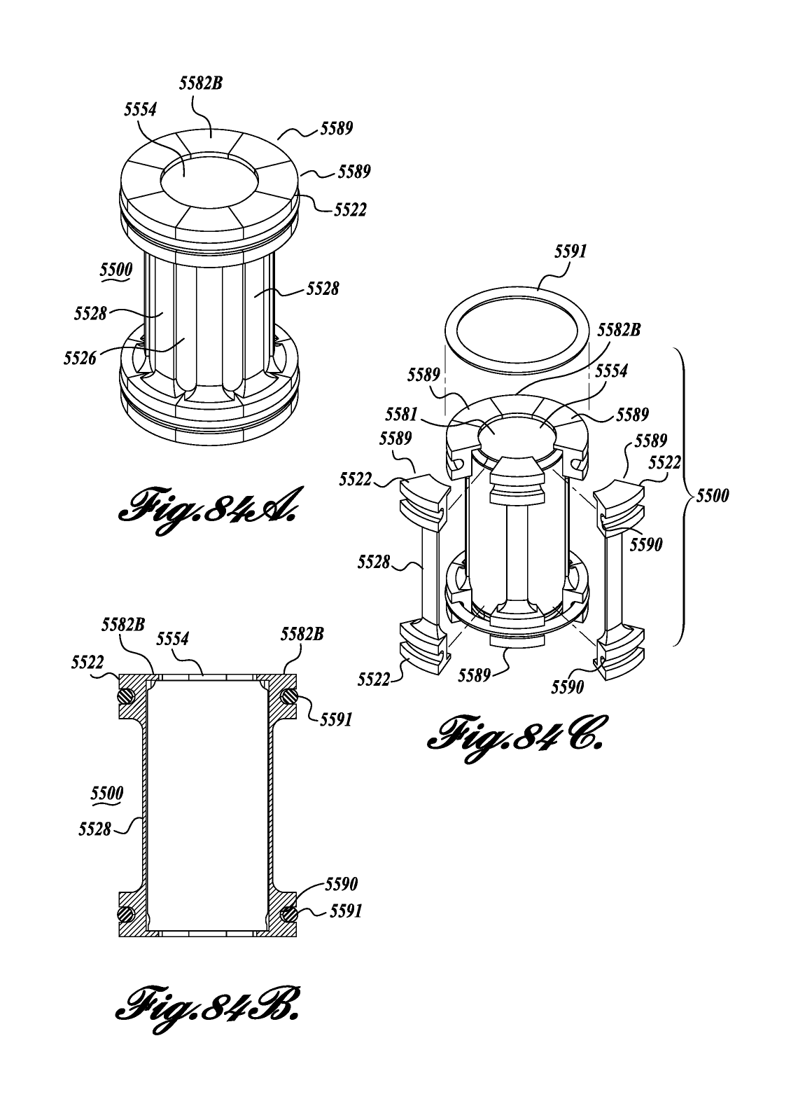

FIGS. 84A, 84B, and 84C illustrate a further embodiment of the present disclosure wherein a carrier 5500 is composed of a plurality of segments 5589. Each segment 5589 includes a rim portion 5522, as well as a stave portion 5528. When the segments 5584 are assembled, the carrier 5500 in construction is similar to carriers described above, including carriers 2200 and 3000, with the exception that at the end 5582B of the rim portion 5522 the inside diameter 5554 of end opening 5554 is smaller than the diameter of the end of the container C, thereby capturing the container C within the carrier. A circumferential slot 5590 is formed to extend around the exterior of the rim 5522 and is sized to receive an expandable garter spring or band 5591 therein for retaining the segments 5589 together to form the unitary carrier 5500. The garter spring/band 5591B can be of various constructions, including composed of elastomeric material, composed of tightly wound spring wire, or of other compositions.

Although the carrier 5500 is illustrated as composed of eight separate segments, the carrier 5500 can be composed of a different number of segments, from a minimum of two to a number greater than shown in FIGS. 84A, 84B, and 84C. Also, the segmented carrier 5500, rather than when assembled resembling carriers 2200 or 3000, can also be in the form of other carriers described above, including, for example, carriers 1900, 2000, and 2100, as well as carriers 4100-4600.

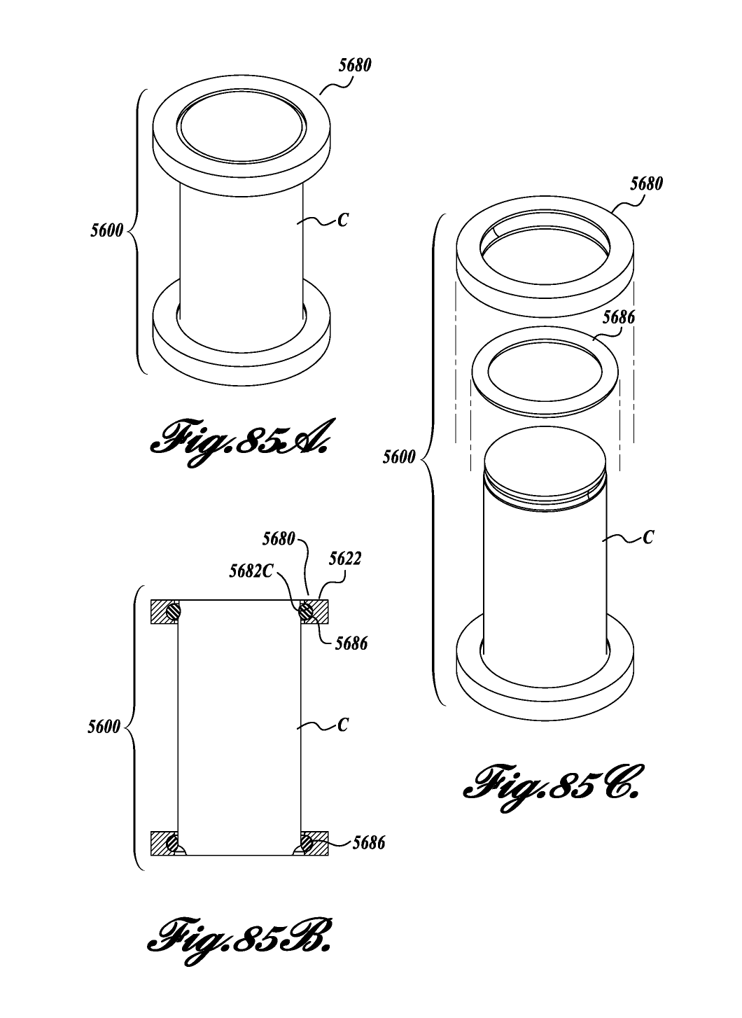

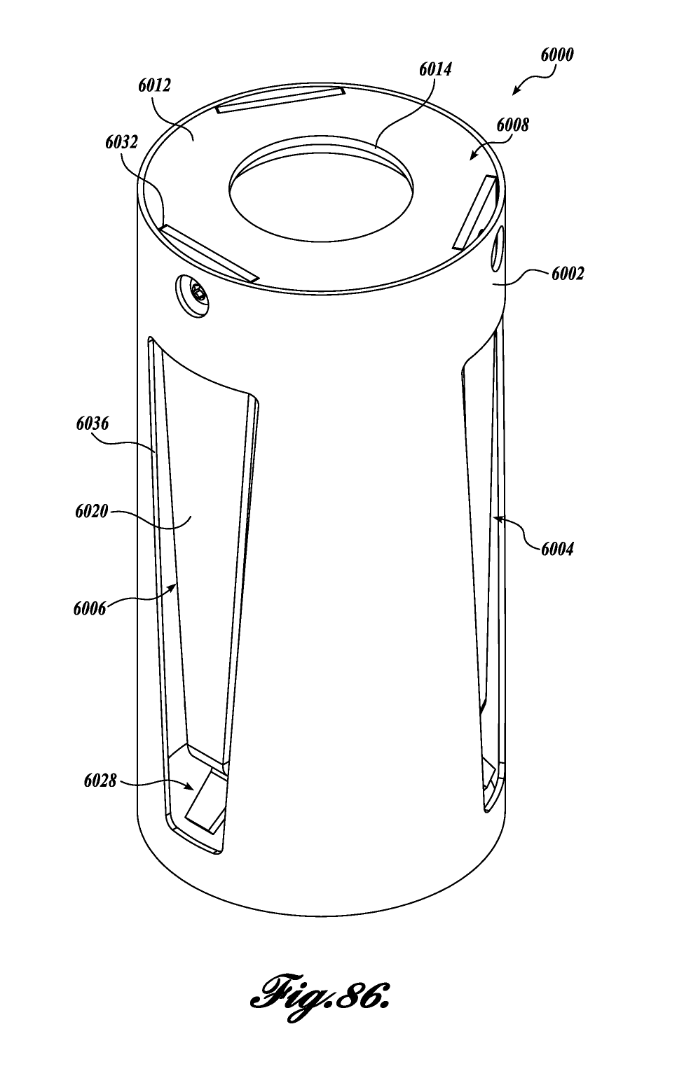

FIGS. 85A, 85B, and 85C illustrate a further embodiment of the present disclosure, wherein the carrier 5600 is shown as composed of two end rim assemblies 5680 sized to engage over the end portions of a container, such as container C. The end rim assemblies can function in a manner similar to the rim portions of carriers described above, for instance carrier 2200. In this regard, the carrier 5600 can roll on the end rim assemblies 5680, thereby to support the container C above surfaces of the processing equipment during the sterilization or other processing of the contents within the container. Also, if properly placed with respect to the container C, the end rim assemblies 5680 can protect the ends of the container from being scratched, dented, or otherwise damaged.

As shown in FIGS. 85A, 85B, and 85C, the end rim assemblies 5680 include a rim portion 5622 constructed with an interior groove 5682C for reception of an O-ring 5686 or similar sealing device therein. The O-ring 5686 bears against the exterior circumferential surface of the container C to retain the end rim assembly engaged with the container C. Although the end rim assembly 5680 is illustrated as including a singular interior groove 5682C, the end rim assembly may be constructed to utilize two or more O-ring seals, for example, as shown in FIGS. 80A, 80B, and 80C. With respect to material composition, the rim portions 5622 may be composed of a high strength plastic, metal, or other durable material.

While several embodiments of the disclosure have been illustrated and described, it will be appreciated that various changes can be made therein without departing from the spirit and scope of the present invention. In this regard, carriers, such as those described and depicted above, can be adapted to hold containers, such as cans and bottles, that are other than of a cylindrical shape, for example, cans and bottles that may be square, hexagonal or octagonal or elliptical in cross-section, and/or smaller in diameter at one end, for example if the container includes a necked-down end portion. In this regard, the body or sleeve portion of the carrier can be shaped to match the cross-sectional shape of the cans or bottles. Also, if the carrier includes circular rims, the end sections of the body or sleeve can be configured to transition the body to the circular rims. In this manner, the carrier is capable of easily rolling along the outer shell of the rotary sterilizer, or along other components of the sterilizer during the sterilization process.

Also, the number of staves, and thus, a corresponding number of slots, may be varied. Further, although the slots and staves are shown as equally spaced around the carrier body, that is not required. Rather, the spacing of the slots and staves can be other than uniformly about the carrier body. Also, the shape of the staves and slots illustrated and described above are only illustrative. The staves and slots can be of other various shapes.

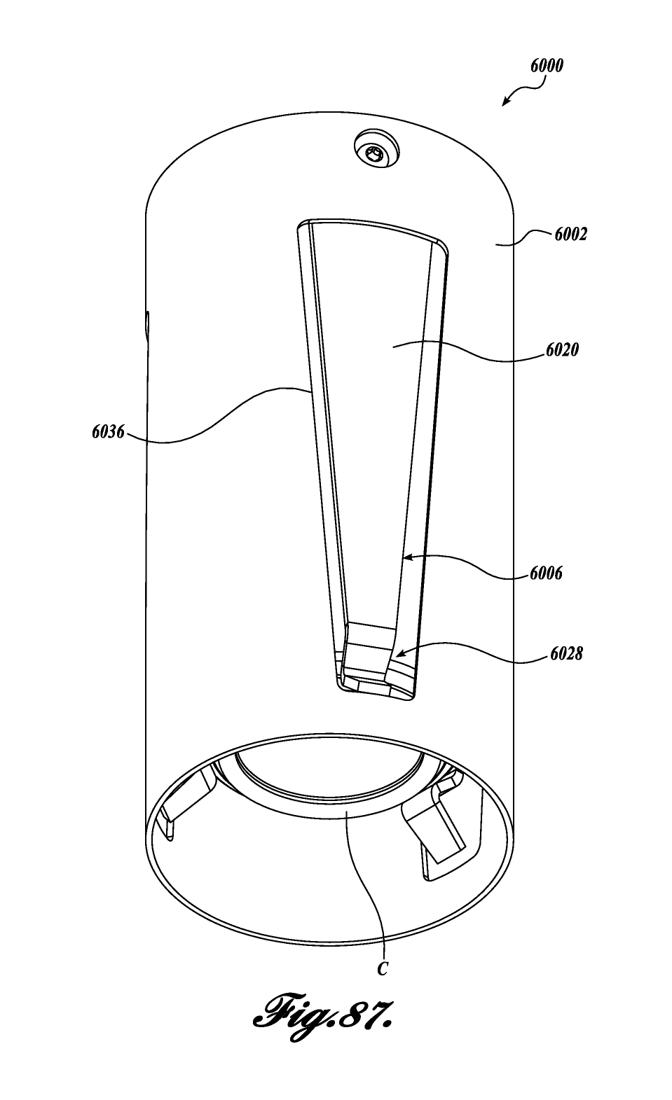

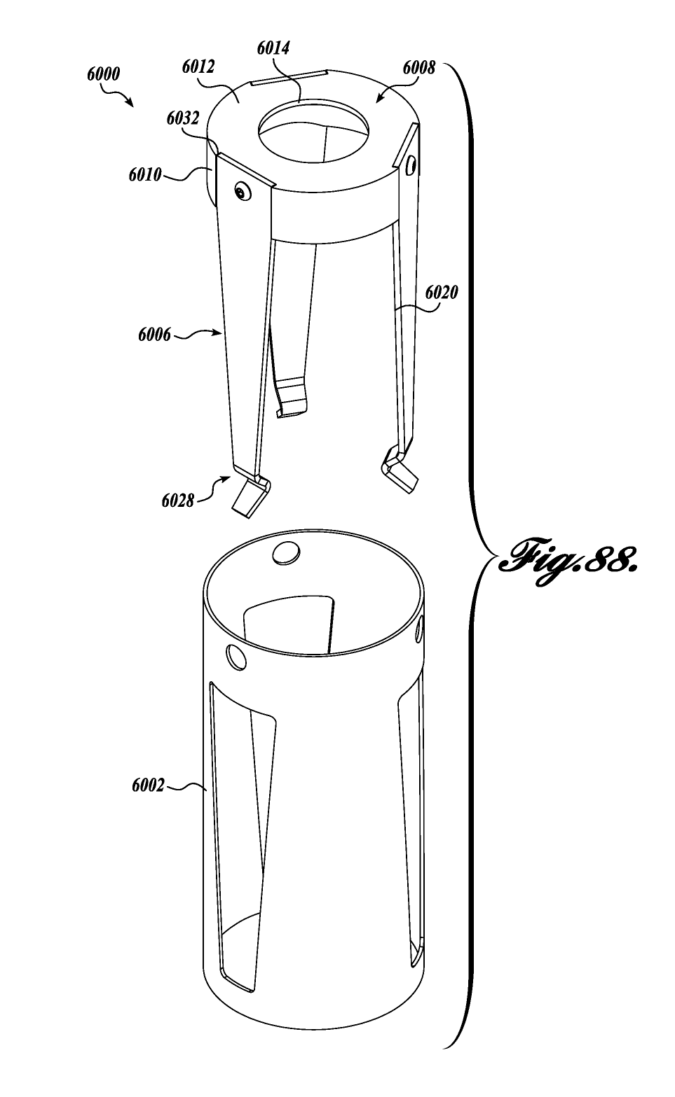

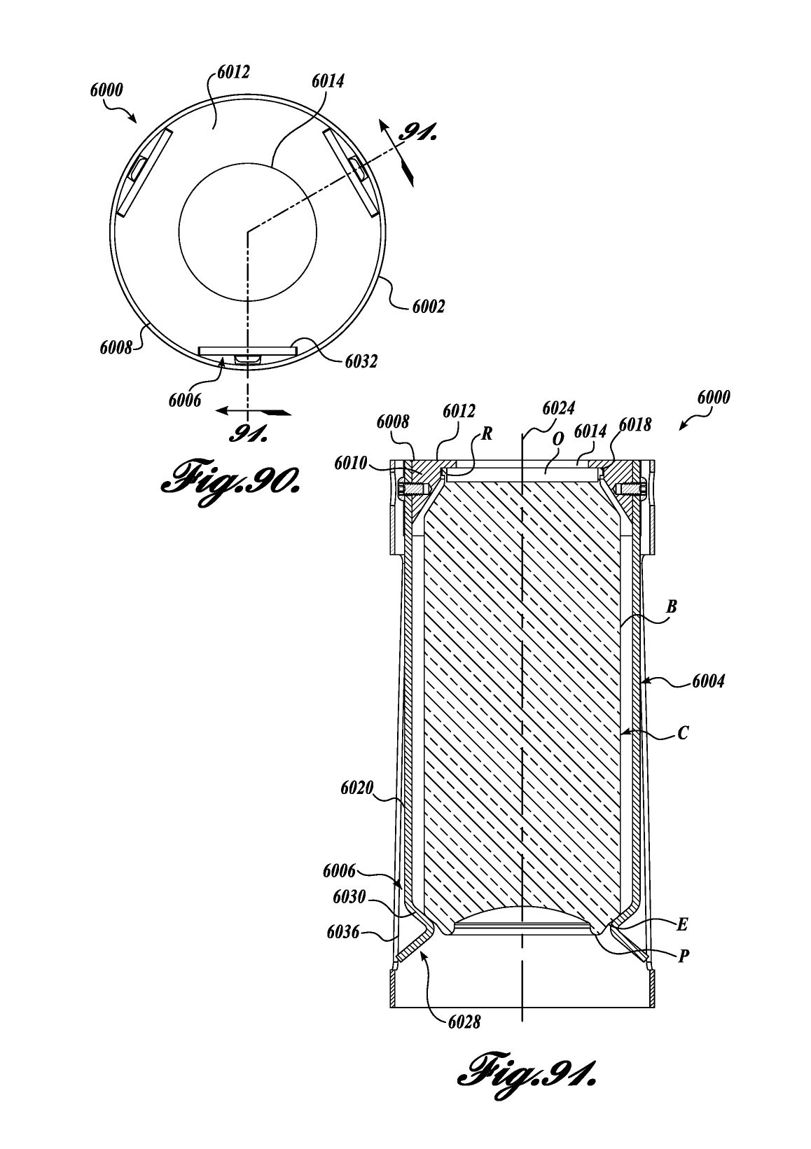

Referring to FIGS. 86-91, another exemplary embodiment of a carrier 6000 formed in accordance with the present disclosure is shown. The carrier 6000 is illustrated as composed of a body 6002 having a length longer than the container C, which is supported and carried by the body 6002. The exterior of the body 6002 is substantially cylindrical in shape. Although not shown, the body 6002 can include rims or chines, for example, at the ends of the body, to add strength to the body as well as provide contact surfaces about which the body may roll. Also, if the exterior of the body is not cylindrical in shape, circular rims or chines provide surface about which the body can roll.

The interior of the body 6002 is shown as being cylindrical in shape and sized to receive the container C with clearance between the exterior of the container and the interior of the body. Of course, the interior of the body can be other than cylindrical in shape, especially if the body is molded from plastic or other moldable or formable material. However, if the body is fabricated from cylindrical material stock, then the interior of the body can simply match the exterior. Regardless, the body 6002 can be formed from a durable material, such as a high strength plastic or metal.

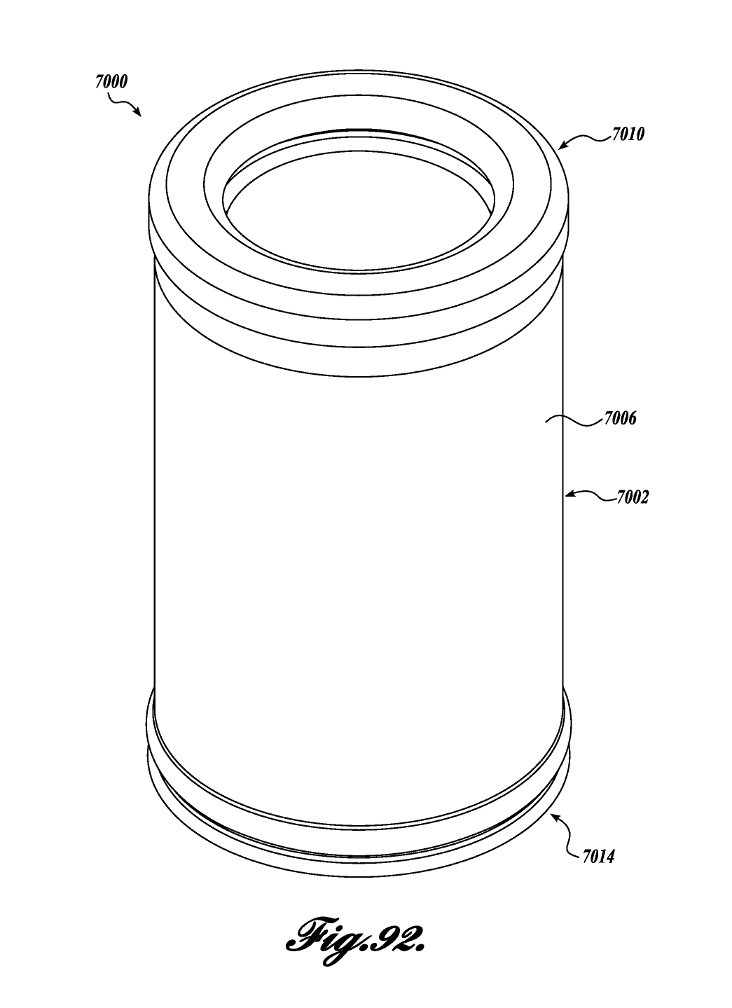

As can be seen in FIG. 91, the inside diameter of the body 6002 is larger than the outside diameter of the container C. Such a difference in diameter allows processing fluid to circulate through the interior of the body 6002 to facilitate heat transfer from the processing fluid to the contents of the container C. Moreover, as with other carriers (such as, for example, carriers 4100 and 4200), openings, perforations, slots, etc., can be provided in the body 6002 to allow processing fluid to enter and/or exit the body 6002 during processing. Such openings may assist in fluid circulation throughout the body 6002, especially if there is not a great deal of clearance between the interior of the body 6002 and the exterior of the container C.

In addition to body 6002, carrier 6000 also includes a retention system 6004 disposed within the body 6002. In the depicted embodiment, the retention system 6004 includes an insert 6008 that is secured within a first open end of the body 6002 for retaining a first end of the container C, and a plurality of flexible fingers 6006 extending from the insert 6008 along a length of the body 6002 for retaining a second opposite end of the container C.

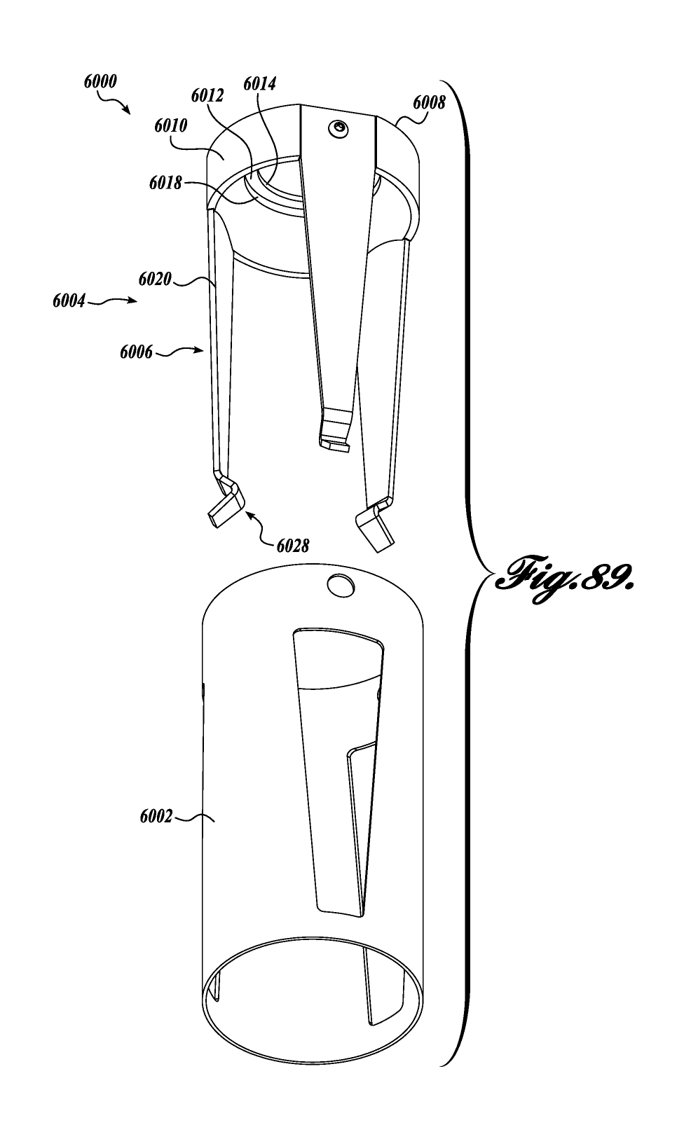

Referring to FIGS. 88-91, the insert 6008 will first be described in detail. The insert 6008 is generally cylindrical in shape or otherwise shaped and sized to be disposed within a first interior end portion of the body 6002. In that regard, the insert 6008 includes a wall section 6010 extending along its length and a transverse end section 6012 enclosing the wall section 6010 (and the body 6002 when the insert 6008 is received therein). The wall section 6010 is substantially cylindrical along its exterior to substantially match the interior shape of the body 6002. Of course, the exterior of the wall section 6010 may instead be any suitable shape to match the correspondingly-shaped interior of the body 6002.

The insert 6008 may be at least temporarily secured within the body 6002 in any suitable manner. For instance, the insert 6008 may be spot or plug welded to the body 6002. In the alternative, the insert 6008 may be secured within the body 6002 through a shrink fit process. As an example, with the body 6002 heated (expanded) and the insert 6008 cooled (shrunk), the body 6002 and the insert 6008 are mated together and the temperature is equalized to define an interference fit therebetween. As yet another alternative, the insert 6008 may be snugly received within the body 6002 to define a friction fit therebetween. In such an alternative embodiment, the insert 6008 can be constructed from a softer, more flexible, material such as a high strength plastic or metal. Also, to help retain the insert 6008 within the body 6002, indentations, grooves, etc., may be formed on one of the exterior surface of the insert 6008 and the interior surface of the body 6002, and correspondingly-shaped features such as protrusions, bumps, etc., can be formed on the other of the exterior surface of the insert 6008 and the interior surface of the body 6002. Of course, numerous systems can be used to retain the insert 6008 within the body 6002. Moreover, it should be appreciated that the insert 6008 may instead be molded into or otherwise formed with the body itself.

As can be best seen by referring to FIG. 91, the interior of the insert 6008 extends upwardly from a bottom edge of the insert 6008 to the transverse end section 6012 and inwardly toward a central axis 6024. In this manner, the wall section 6010 of the insert 6008 has a depth for receiving fasteners or other devices used to secure the flexible fingers 6006 to the insert 6008, as will be described further below. The interior of the wall section 6010 extends upwardly and inwardly toward a central opening 6014 defined in the transverse end section 6012. The central opening 6014 is configured to allow heat to more efficiently transfer between the container C and the sterilizer. Of course, any suitable opening or plurality of openings may instead be defined in the insert 6008 for appropriate heat transfer.

As noted above, the insert 6008 is configured to retain a first end of the container C within the body 6002 when the insert 6008 is secured within a first open end of the body 6002. In that regard, an annular shoulder 6018 is defined on the interior of the insert 6008 for receiving and retaining the first end of the container C. The annular shoulder 6018 surrounds the central opening 6014 and is defined between the inwardly sloped interior of the wall section 6010 and the transverse end section 6012. The annular shoulder 6018 is shaped to substantially correspond to and receive the first end of the container C.

In the illustrated embodiment, the container C is a 2-piece can, such as an aluminum can having a generally cylindrical body B and an upper annular rim R defined by a cover O secured to the body B through double seaming technology or the like. The annular shoulder 6018 is configured to capture and retain the upper annular rim R of the container. More specifically, the inner diameter of the annular shoulder 6018 is substantially the same size as the outer diameter of the upper annular rim R. In other words, there is a close tolerance between the insert 6008 and the upper annular rim R. The close tolerance between the inner diameter of the annular shoulder 6018 and the outer diameter of the upper annular rim R helps to distribute impact stresses on the carrier 6000.

Moreover, with the insert 6008 engaging the entire upper annular rim R with the smooth, continuous interior annular shoulder 6018, any damage to the rim R during processing is minimized. In other words, the insert 6008 does not damage (and protects) the upper annular rim R of the container C when engaged therewith. As can be appreciated by one of ordinary skill, a 2-piece can or similar container having a scratched or damaged upper rim is not desired because it is aesthetically unpleasant to a consumer. Accordingly, the insert 6008 helps prevent any damage to the upper end of the container during processing.

It should be appreciated that the interior of the insert 6008 may instead be any suitable shape to correspond to a first end of any suitable container. For instance, the insert 6008 may instead be shaped on its interior to correspond to a 3-piece can, a bottle-necked container, etc.

The insert 6008 is made from a suitable material to help distribute impact stresses on the carrier 6000 and minimize damage to the upper annular rim R during processing. For instance, the insert 6008 may be made from stainless steel or another high strength and heat tolerant metal or plastic.

The flexible fingers 6006 of the retention system 6004 configured to retain a second end of the container C within the body 6002 will now be described in detail. As can best be seen by referring to FIGS. 88, 89, and 91, the retention system 6004 includes a plurality of fingers 6006 extending from the insert 6008 along a length of the body 6002 for engaging and retaining the second end of the container C within the body. Each finger 6006 includes a flexible elongated section 6020 that extends from the insert 6008 generally longitudinally and slightly diagonally inwardly toward the central axis 6024 (see FIG. 91) of the body 6002 along a length of the body 6002.

The elongated section 6020 tapers in width or otherwise becomes more deformable near its distal end for passing over or removably retaining a container C between the plurality of fingers 6006. However, it should be appreciated that the elongated section 6020 may instead decrease in thickness or otherwise change in material gauge to define a sufficient amount of deformability or flexibility in the distal portions of the elongated section 6020. As such, the elongated section 6020 may be deformed outwardly away from the central axis 6024 and a container C may be passed upwardly between the plurality of fingers 6006 toward the insert 6008 for disposing the container C within the carrier 6000.

More specifically, the fingers 6006 may be moved between a first position, wherein the fingers 6006 are spaced from the container C to receive the container into the body 6002, and a second position, wherein the fingers 6006 hold the container within the body. The elastic properties of the fingers 6006 bias the fingers 6006 toward the second position such that the fingers 6006 impose a force on the container C to retain the container within the body 6002.

Each finger 6006 includes at its distal end a tab portion 6028 that is configured to bear against a bottom portion of the container C when the container C is received between the fingers 6006 and within the upper interior portion of the insert 6008. The tab portion 6028 may be any suitable shape and configuration for appropriately bearing against a bottom portion of the container C for retaining the container C within the body 6002 without damaging the container C during processing.

In the depicted embodiment, the lower end of the container C (opposite the annular rim R) is enclosed and includes an inwardly and downwardly annular sloped end surface E terminating in an annular protrusion P, with both the annular sloped end surface E and the annular protrusion P substantially concentric about central axis 6024. The tab portion 6028 is substantially V-shaped in cross-section with the apex of the V extending toward the central axis 6024. In this manner, at least a portion of the annular sloped end surface E may bear against or rest against an upper portion 6030 of the V-shaped tab portion 6028 when disposed within the carrier 6000.

It should be appreciated that the tab portion 6028 may instead be any other suitable shape or configuration for bearing against another bottom portion of the container C or against the bottom portions of other various-shaped containers C. Moreover, it should be appreciated that the tab portion 6028 may be made from a different material than the elongated section 6006, such as a deformable or pliable material that will not scratch or damage the container C. In the alternative, the tab portion 6028 may be coated with or otherwise covered with a non-abrasive material, such as rubber.

The tab portions 6028 of the fingers 2006 are collectively configured to center the container C within the body 6002, as well as to substantially prevent any longitudinal or axial movement of the container C when positioned against the insert 6008. Moreover, the tab portions 6028 are positioned intermediate the end of the body 6002, such that the container C does not protrude from the body 6002 when retained therein by the fingers 6006.

The fingers 6006 can be constructed from spring steel or other material which allows the fingers 6006 to be flexible. In this manner, the fingers 6006 are retractable to allow the container C to be loaded into and out of the carrier 6000 without bearing against the container C during the loading and unloading process so as not to damage container C. Also, the spring steel construction causes the fingers 6006 to be resilient and bear against the container C once the retracting force is removed from the fingers 6006 and they are allowed to bear against the container C (when moving from the first to second position). It should be appreciated that the fingers 6006 can instead be constructed of other materials that provide other characteristics similar to spring steel, for example, a flexible plastic-based material. Moreover, as noted above with respect to the tab portions 6028, some or all portions of the fingers 6006 can be coated with a non-abrasive material, such as rubber. In this manner, any contact that the fingers 6006 have with the container C during the loading or unloading process minimizes any scratching or other damage to the container C.

The proximal end of the fingers 6006 is secured to the insert 6008 for defining the retention system 6004 that is disposable within the body 6002. The plurality of fingers 6006 are attached at their proximal ends to the insert 6008 through hardware members, such as screws, bolts, rivets, etc., which can extend through clearance holes form in the body 6002 (not labeled). It should be appreciated that the fingers 6006 may be secured to the insert 6008 in any other suitable manner, such as with adhesive, weldments, etc.

The fingers 6006 may be received within finger receiving cavities or recesses 6032 defined along the exterior surface of the insert 6008. In this manner, the overall shape of the combined finger 6006 and insert 6008 assembly is cylindrical for fitting within the body 6002. Each recess 6032 extends longitudinally along the cylindrical wall section 6010 and has a predetermined depth for receiving a finger 6006 in a manner so that the finger 6006 does not obstruct the insertion of the retention system 6004 within the body 6002.

The recesses 6032 are spaced equidistant around the circumference of the insert 6008 to space the fingers 6006 substantially equally around the insert 6008 and around the body 6002 when received therein. In the depicted embodiment, the insert 6008 includes three recesses 6032 for receiving three fingers 6006. However, it should be appreciated that any other suitable number of recesses 6032 and fingers 6006 may instead be used, such as two, four, five, etc. For instance, less than or more than three fingers 6006 may be used if the container C is of a shape other than cylindrical. For example, if the container is generally square-shaped, four fingers 6006 may be utilized. Also, if the fingers 6006 including the tab portion 6028 are constructed differently from that described above, it is possible that even a singular finger or tab structure may be utilized with the carrier 6000.

As can be seen in FIGS. 86, 87 and 91, with the retention system 6004 disposed within the body 6002, a container C may be loaded into the retention system 6004 to secure the container C within the carrier 6000 for processing when the fingers 6006 are in the first position. In particular, the retention system 6004 is loaded within the body 6002 such that the insert 6008 fits snugly in a first or upper end of the body 6002. Preferably, the insert 6008 is positioned within the first end of the body 6002 such that the transverse section 6012 of the insert 6008 is substantially flush with an upper edge of the body 6002. In an alternative configuration, the retention system 6004 is integrally formed with the body 6002.

With the insert 6008 received within the body 6002, the fingers 6006 extend longitudinally along the length of the body 6002 and slightly inwardly toward the central axis 6024. As shown in FIG. 91, the distal ends of the fingers 6006 defined by tab portion 6028 terminate intermediate of the second or bottom end of the body 6002. In this manner, when a container C is received within the retention system 6004 of the carrier 6000, the container C is not exposed or it does not otherwise protrude from the body 6002. However, it should be appreciated that in some embodiments, a portion of container C may protrude from the body 6002, such as a container having an elongated bottleneck or similar.

With the retention system 6004 (defined by the insert 6008 and the fingers 6006) disposed within the body 6002, the fingers 6006 may be retracted away from the central axis 6024 into the first position to load or unload a container C from within the retention system 6004. In that regard, the body 6002 includes a plurality of openings 6036 that correspond in position to a majority of the length of the fingers 6006. Specifically in the embodiment depicted, the body 6002 includes three openings corresponding to the three fingers 6006. The openings 6036 extend along a portion of the length of the body 6002 to provide access to the corresponding fingers 6006. Through the openings 6036, the fingers 6006 may be retracted away from the central axis 6024 into the first position to load or unload the container C therefrom. A retractor or other device may be used to retract the fingers 6006 away from the central axis 6024 for the loading and unloading process.

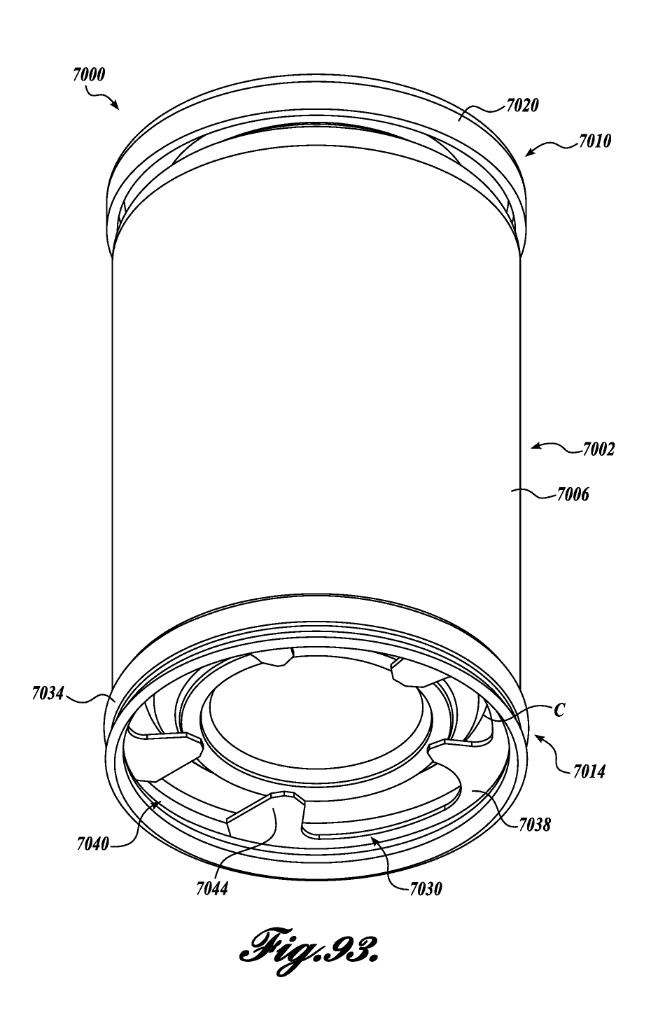

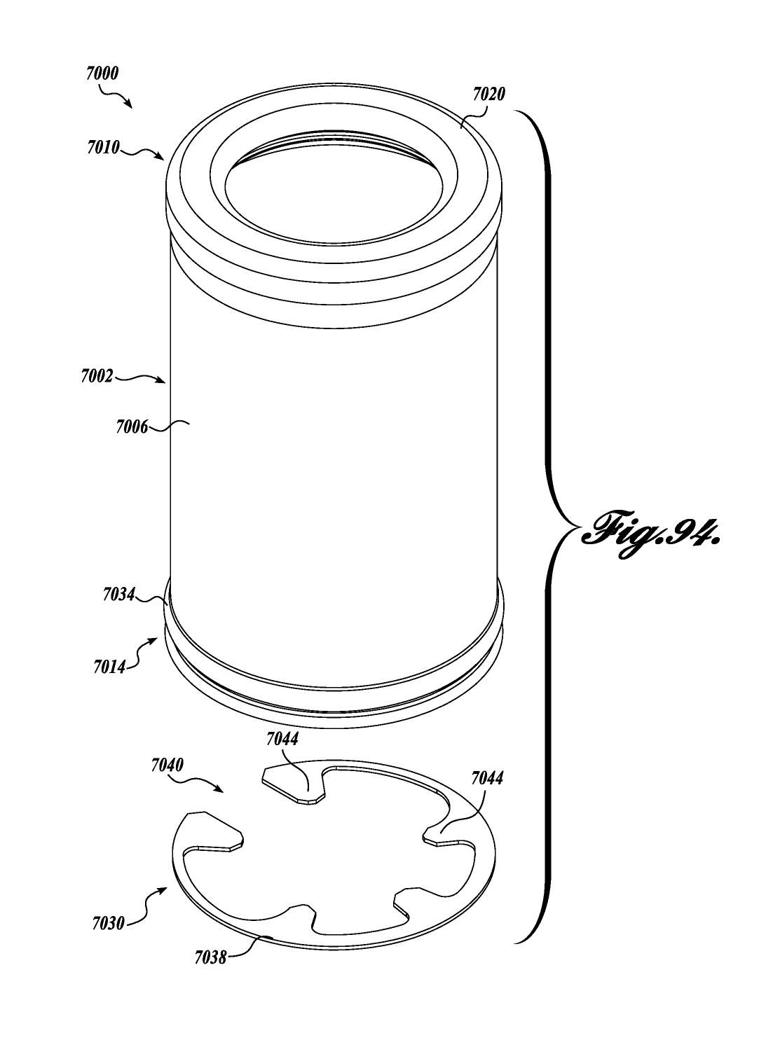

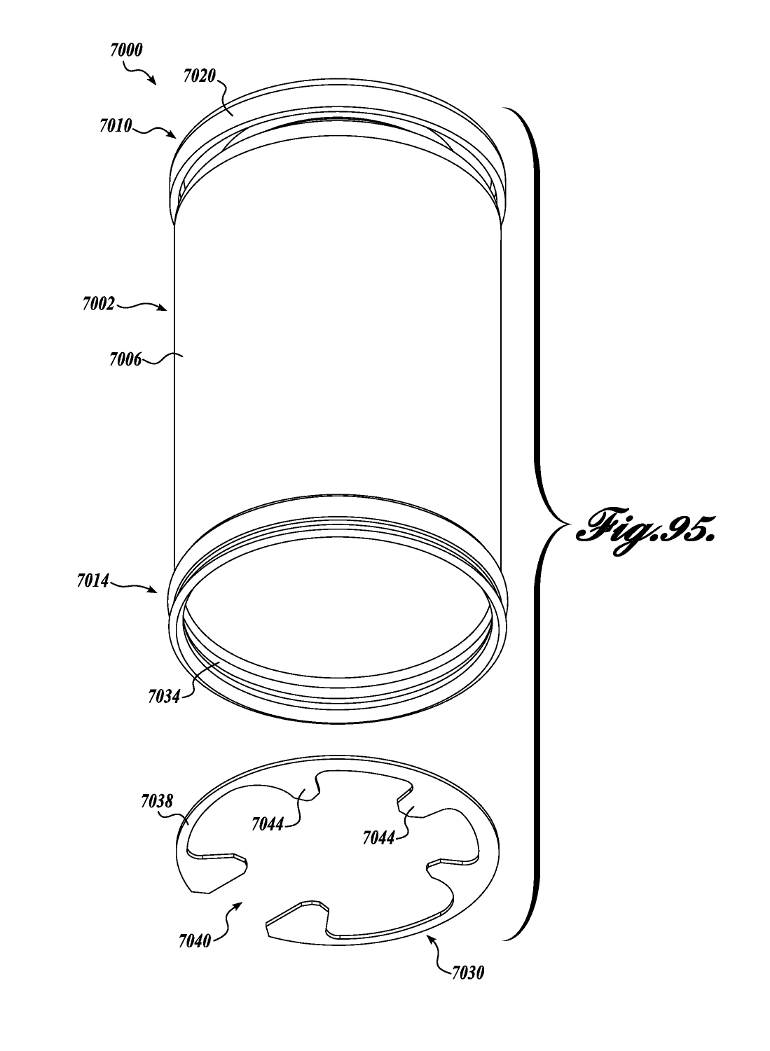

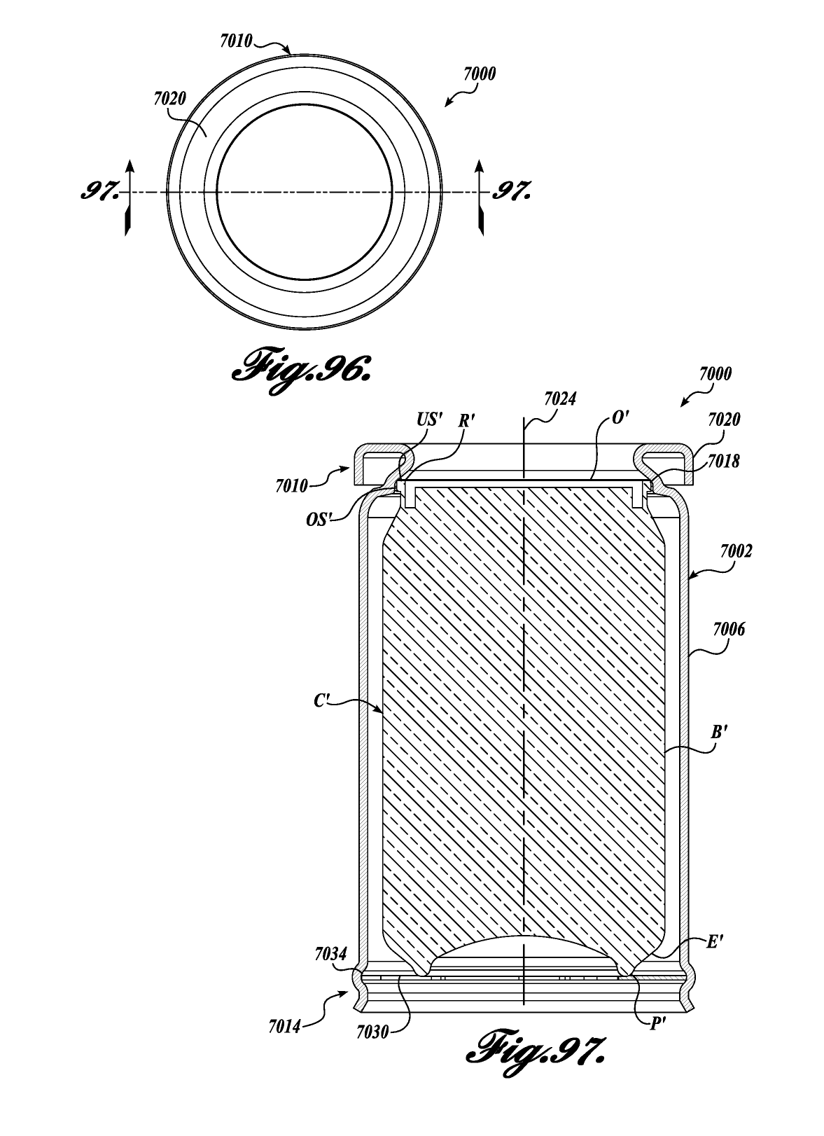

Referring to FIGS. 92-97, another exemplary embodiment of a carrier 7000 formed in accordance with the present disclosure is shown. The carrier 7000 is illustrated as composed of a body 7002 having a length longer than the container C′, which is supported and carried by the body 7002. The body 7002 includes a cylindrical portion 7006 extending along a majority of the length of the body 7002, a first end portion 7010 defined at a first end of the cylindrical portion 7006, and a second end portion 7014 defined at a second end of the cylindrical portion 7006 opposite the first end portion 7010.

The cylindrical portion 7006 has a generally cylindrical exterior and interior, and an inside diameter larger than the container C′. More specifically, the interior of the body 7002 is shown as being cylindrical in shape and sized to receive the container C′ with clearance between the exterior of the container and the interior of the body. Of course, the interior of the body can be other than cylindrical in shape, especially if the body is molded from plastic or other moldable or formable material. However, if the body is fabricated from cylindrical material stock, then the interior of the body can simply match the exterior. Regardless, the body 7002 can be formed from a durable material, such as a high strength plastic or metal. Moreover, as with other carriers (such as, for example, carriers 4100 and 4200), slots, holes, or other types of openings can be formed in the body 7002 to facilitate heat transfer with the contents of the container C′.

Although not shown, the body 7002 can include rims or chines, for example, at the ends of the body, to add strength to the body as well as provide contact surfaces about which the body may roll. Also, if the exterior of the body is not cylindrical in shape, circular rims or chines provide a surface about which the body can roll.

Referring to FIG. 97, the first end portion 7010 of the body 7002 will now be described in detail. The first end portion 7010 of the body 7002 is generally configured to capture and retain a first end of the container C′ within the body 7002. In the illustrated embodiment, the container C′ is a 2-piece can substantially identical to the 2-piece can C shown in FIG. 91. As such, like parts have been identified with like reference letters except with a prime′.

The first end portion 7010 of the body 7002 is generally configured to capture and retain the upper annular rim R′ of the 2-piece can C′. In that regard, an interior annular shoulder 7018 is defined between the cylindrical portion 7006 and the first end portion 7010. The interior annular shoulder 7018 is configured to engage and the upper corner of the annular rim R′ defined by the outer surface OS' of the upper annular rim R′ and the upper surface US' of the upper annular rim R′. In this manner, the upper and outer surfaces US' and OS' of the upper annular rim R′ are substantially prevented from any damage during processing.

As can be appreciated and as noted above with respect to carrier 6000, a 2-piece can or a similar container having a scratched or damaged upper rim is not desired because it is aesthetically unpleasant to a consumer. Moreover, the upper and outer surfaces US' and OS' of the upper annular rim R′ are highly visible to the consumer when consuming the contents of the container C′. Accordingly, by instead engaging the upper corner of the upper annular rim R′, damage to the upper end of the container is minimized during processing.

The first end portion 7010 of the body 7002 is also generally configured to provide support for the carrier 7000 as it is processed in the sterilizer. As noted above, chines or rims may be provided on the body 7002 to provide additional support for the body as it travels through the sterilizer. In that same regard, the first end portion 7010 includes a folded-over edge 7020 that helps provide stability and support to the body 7002 as it is processed. More specifically, the folded-over edge 7020 helps provide axial and radial impact resistance to the first end of the carrier 7000 as it travels through the sterilizer.

The second end portion 7014 of the body 7002 will now be described in detail. The second end portion 7014 of the body 7002 is configured to receive a retention system for engaging the second end of the container C′ and retaining the container C′ within the body 7002. In the depicted embodiment, the second end portion 7014 is generally configured to removably receive a retaining member, such as a clip 7030 that is engageable with and bears against the bottom of the container C′ when received within the carrier 7000. In that regard, an interior annular groove 7034 is defined in the second end portion 7014 that is configured to removably receive the clip 7030. The annular groove 7034 is substantially concentric with a center longitudinal axis 7024 of the body 7002 and positioned is axially along the body 7002 (on the second end portion 7014) such that the clip 7030 may engage the annular protrusion P′ on the bottom of the container C′. The annular groove 7034 may be any suitable cross-sectional shape and size to help ease the clip 7030 into and out of the groove 7034.

The clip 7030, which can best be seen in FIGS. 93-95, has a clip body 7038 that is generally C-shaped and has a circular outer diameter that is somewhat larger than the interior diameter of the annular groove 7034 in an undeformed state. With the clip body 7038 being C-shaped, the body includes a gap 7040 that may be decreased or increased in size to decrease or increase the size of the outer diameter of the clip body 7038. In other words, the clip 7030 may be squeezed to decrease the gap 7040 (decreasing the clip outer diameter) for moving the clip 7030 into and out of the annular groove 7034. When received within the annular groove 7034, the clip 7030 may be released back toward its original shape and size. With the interior diameter of the annular groove 7034 being smaller than the outer diameter of the clip body 7038 in an undeformed state, the clip 7030 is retained within the annular groove 7034 when released.

The clip 7030 is made from an elastic material having a suitable yield strength such that it may be deformed (decreased in diameter) for moving into and out of the annular groove 7034 and released back toward its original state for being retained within the annular groove 7034. Any suitable material may be used, such as spring steel. It should be appreciated that the clip 7030 can instead be constructed of other materials that provide other characteristics similar to spring steel, for example, a flexible plastic-based material. Moreover, some or all portions of the clip 7030 can be coated with a non-abrasive material, such as rubber, to help prevent any scratching or other damage to the container C′ when engaged by the clip 7030.

The clip 7030 also includes a plurality of radial projections 7044 extending from the clip body 7038 toward the center of the clip. The radial projections 7044 are co-planar with the clip body 7038 and are of a suitable length such that they engage the annular protrusion P′ of the container C′ when the container C′ is disposed within the carrier body 7002 and the clip 7030 is disposed within the annular groove 7034. Moreover, the radial projections 7044 are somewhat pointed or taper in width as they extend radially inwardly so as to minimize the contact area with the annular protrusion P′. It should be appreciated, however, that the radial projections 7044 may instead extend from the clip body 7038 at an angle and may be any suitable shape and size to appropriately engage a bottom portion of a container. For instance, if the container is a bottle or another type of container different from a 2-piece can, the shape, size, location, etc., of the radial projections 7044 may be adjusted accordingly.

In the depicted embodiment, the clip 7030 includes five radial projections 7044 spaced substantially evenly around the circumference of the clip body 7038. However, any suitable number of radial projections 7044 and any suitable spacing between projections may be used.

It can be appreciated that by using radial projections 7044 to engage the annular protrusion P′, rather than by extending the clip body 7038 radially inwardly to engage the annular protrusion P′, the clip 7030 remains sufficiently flexible to be deformed into engagement with the annular groove 7034. Moreover, the radial projections 7044 provide less contact with the annular protrusion P′ that would otherwise occur if the entire clip body 7038 engaged the annular protrusion P′. Accordingly, any scratching or damage caused by the contact is minimized.

In use, the clip 7030 is secured within the annular groove 7034 after the container C′ is disposed within the body 7002 such that the top of the container, and specifically, the annular rim R′ is engaged with the interior of the first end portion 7010. With the container C′ secured within the carrier 7000 in this manner, the more visible annular rim R′ is not scratched or damaged, even minimally, by the clip 7030. Rather, the container C′ can be positioned within the carrier 7000 such that the more significant contact occurs on the bottom of the container C′. Moreover, as with other carriers, the body 7002 is sufficiently large in diameter, and the container C′ is positioned within the carrier such that the sides of the container C′ are not touched by the body 7002. Accordingly, the carrier 7000 is suitable for transporting a container through a sterilizer while minimizing any scratching or other damage to the container during processing.

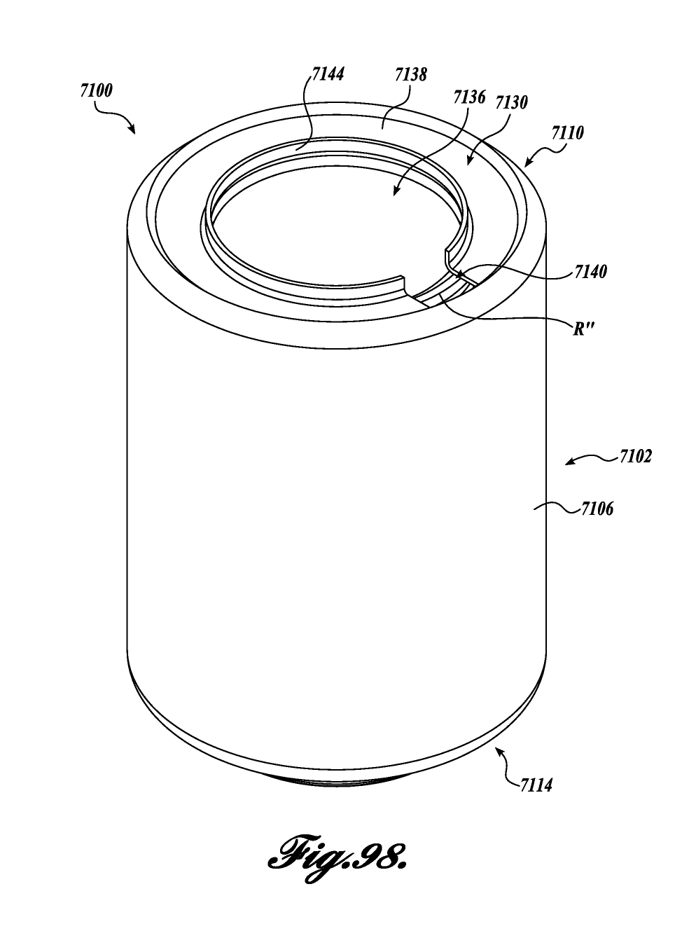

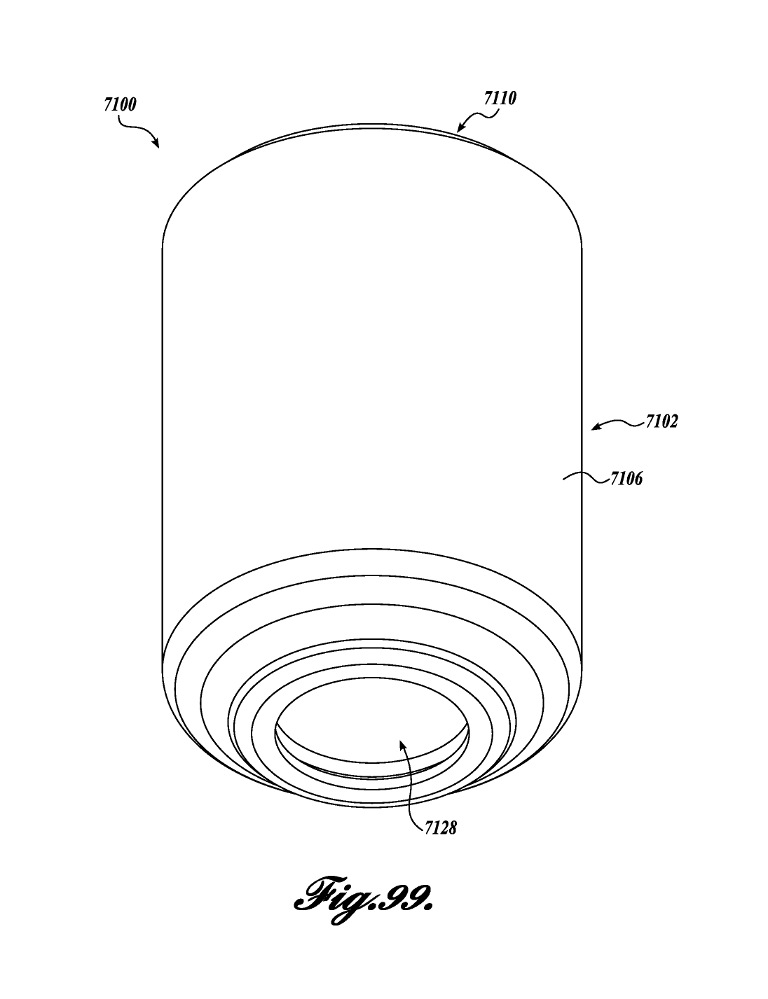

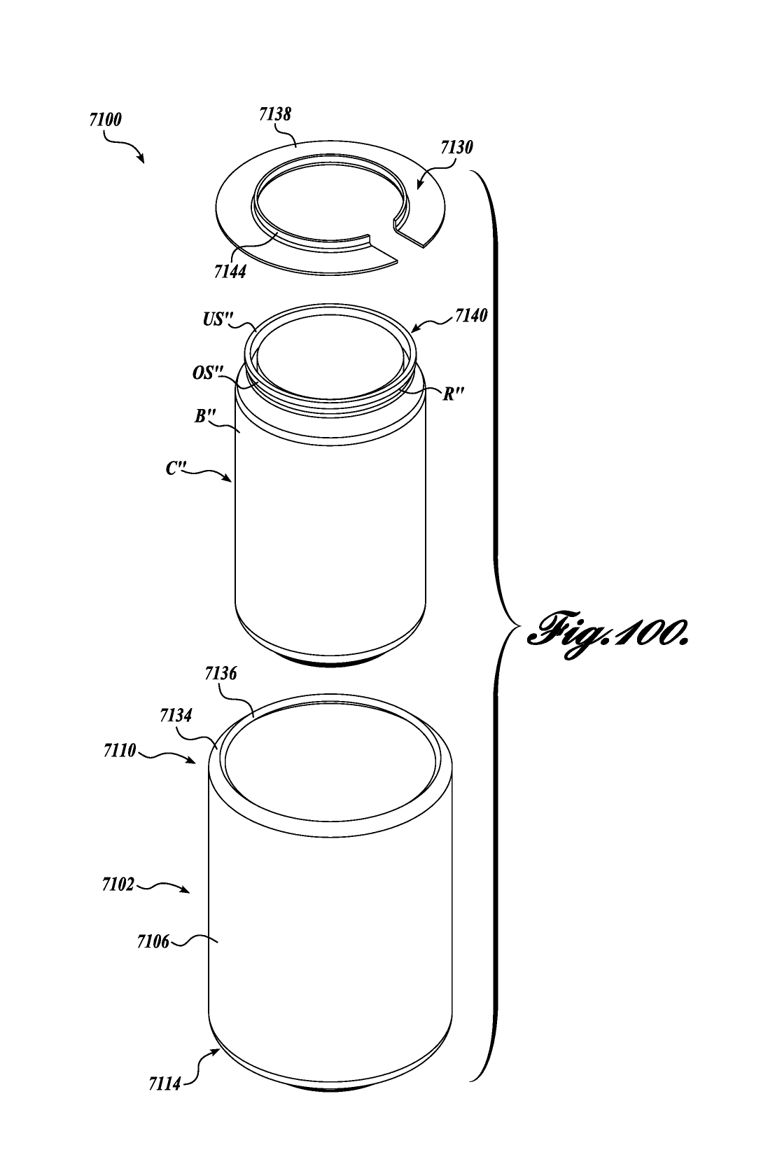

Referring to FIGS. 98-102, another exemplary embodiment of a carrier 7100 formed in accordance with the present disclosure is shown. The carrier 7100 is illustrated as composed of a body 7102 having a length longer than the container C″, which is supported and carried by the body 7102. The body 7102 includes a cylindrical portion 7106 extending along a majority of the length of the body 7102, a first end portion 7110 defined at a first end of the cylindrical portion 7106, and a second end portion 7114 defined at a second end of the cylindrical portion 7106 opposite the first end portion 7110.

The cylindrical portion 7106 has a generally cylindrical exterior and interior, and an inside diameter larger than the container C″. More specifically, the interior of the body 7102 is shown as being cylindrical in shape and sized to receive the container C″ with clearance between the exterior of the container and the interior of the body. Of course, the interior of the body can be other than cylindrical in shape, especially if the body is molded from plastic or other moldable or formable material. However, if the body is fabricated from cylindrical material stock, then the interior of the body can simply match the exterior. Regardless, the body 7102 can be formed from a durable material, such as a high strength plastic or metal. Moreover, as with other carriers (such as, for example, carriers 4100 and 4200), slots, holes, or other types of openings can be formed in the body 7102 to facilitate heat transfer with the contents of the container C″.

Although not shown, the body 7102 can include rims or chines, for example, at the ends of the body, to add strength to the body as well as provide contact surfaces about which the body may roll. Also, if the exterior of the body is not cylindrical in shape, circular rims or chines provide surface about which the body can roll.

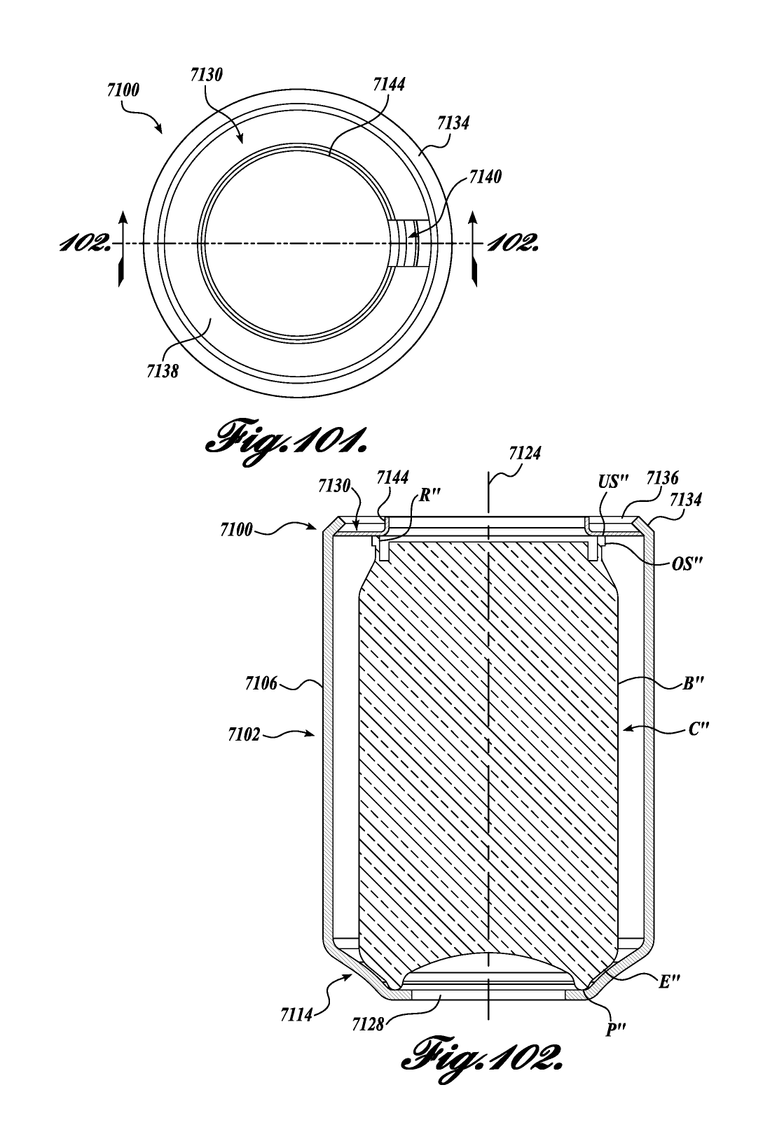

Referring to FIG. 102, the second end portion 7114 of the body 7102 will first be described in detail. The second end portion 7114 of the body 7102 is generally configured to capture and retain a second end of the container C″ within the body 7102. In the illustrated embodiment, the container C″ is a 2-piece can substantially identical to the 2-piece cans C and C′ shown in FIGS. 91 and 97. As such, like parts have been identified with like reference letters except with a double prime″.

The second end portion 7114 of the body 7102 is generally configured to capture and retain the container C″ in a centrally located position (related to a central axis 7124) within the body. More specifically, the second end portion 7114 of the body 7102 is shaped to receive the bottom of the container C″ defined by the inwardly and downwardly annular sloped end surface E″ and the annular protrusion P″. In that regard, the second end portion 7114 is generally shaped to match the shape of the bottom of the container C″. In this manner, the second end portion 7114 secures and retains the container C″ in the centrally located position. A central bottom opening 7128 may also be defined in the second end portion 7114 to help facilitate heat transfer with the contents of the container C″.

The first end portion 7110 of the body 7102 will now be described in detail. The first end portion 7110 is generally configured to removably receive a retaining member, such as a clip 7130, for bearing against the first end of the container C″ when received within the carrier 7100. In that regard, the first end portion 7110 includes a tapered annular end 7034 that is configured to position the clip 7130 against the first end of the container C″. More specifically, the tapered annular end 7134 decreases the diameter of the body 7102 at its first or upper end, such that when a clip 7130 having an outer diameter larger than the tapered annular end 7134 is disposed in the first end of the body, the clip 7130 is retained in is axial position within the body 7102. In other words, the clip 7130 is secured in its position between the first end of the container C″ and the tapered annular end 7134 of the body. The tapered annular end 7134 surrounds a top central opening 7136 to help facilitate heat transfer with the contents of the container C″.

The clip 7130 has a clip body 7138 that is generally C-shaped and has a circular outer diameter that is somewhat larger than the interior diameter of the body 7102 (and the tapered annular end 7134) in an undeformed state. With the clip body 7138 being C-shaped, the body includes a gap 7140 that may be decreased or increased in size to decrease or increase the size of the outer diameter of the clip body 7138. In other words, the clip 7130 may be squeezed to decrease the gap 7140 (decreasing the clip outer diameter) for moving the clip 7130 into and out of the body 7102. When received within the body 7102, the clip 7130 may be released back toward its original shape and size. With the interior diameter of the body 7102 (and the tapered annular end 7134) being smaller than the outer diameter of the clip body 7138 in an undeformed state, the clip 7130 is retained within the body 7102 when released. Moreover, when positioned against the container C″, the clip 7130 is retained in its axial position between the container C″ and the tapered annular end 7134.

The clip 7130 is made from an elastic material having a suitable yield strength such that it may be deformed (decreased in diameter) for moving into and out of the body 7102 and released back toward its original state for being retained within the body 7102. Any suitable material may be used, such as spring steel. It should be appreciated that the clip 7130 can instead be constructed of other materials that provide other characteristics similar to spring steel, for example, a flexible plastic-based material. Moreover, some or all portions of the clip 7130 can be coated with a non-abrasive material, such as rubber, to help prevent any scratching or other damage to the container C″ when engaged by the clip 7130.

Unlike clip 7030 shown in FIGS. 93-97, the clip 7130 does not include radial projections extending from the clip body 7138. Rather, the clip body 7138 merely extends radially toward the center of the clip a predetermined distance such that the body 7138 itself is engageable with the container C″. Of course, the clip 7130 may instead include radial projections or any other suitable configuration for appropriately engaging the container C″.

In the depicted embodiment, the clip body 7138 extends toward the central axis 7124 a predetermined distance until it engages the upper surface US″ of the upper annular rim R″ of the container C″. The clip body 7138 terminates in a substantially transverse annular flange 7144 extending upwardly from the clip body 7138. The annular flange 7144 is graspable by the user (or by an automated or semi-automated tool or equipment) to deform the clip 7130 for moving it into and out of the body 7102. In that regard, the clip 7130 may instead include any other mechanism for grasping and deforming the clip.

In use, the clip 7130 is secured within the first end portion 7110 of the body 7102 after the container C″ is disposed within the body 7102 such that the bottom of the container, and specifically, the annular sloped end surface E″ and the annular protrusion P″ are engaged with the interior of the second end portion 7114. With the container C″ secured within the carrier 7100 in this manner, the clip 7130 may be disposed within the body 7102 such that it is positioned axially between the upper annular rim R″ of the container and the tapered annular end 7134. The clip 7130 therefore secures the container C″ within the body 7102 of the carrier 7100 and prevents the container from moving within the carrier during processing.

Moreover, as with other carriers, the body 7102 is sufficiently large in diameter, and the container C″ is positioned within the carrier such that the sides of the container C″ are not touched by the body 7102. Accordingly, the carrier 7100 is suitable for transporting a container through a sterilizer while minimizing any scratching or other damage to the sides of the container during processing.

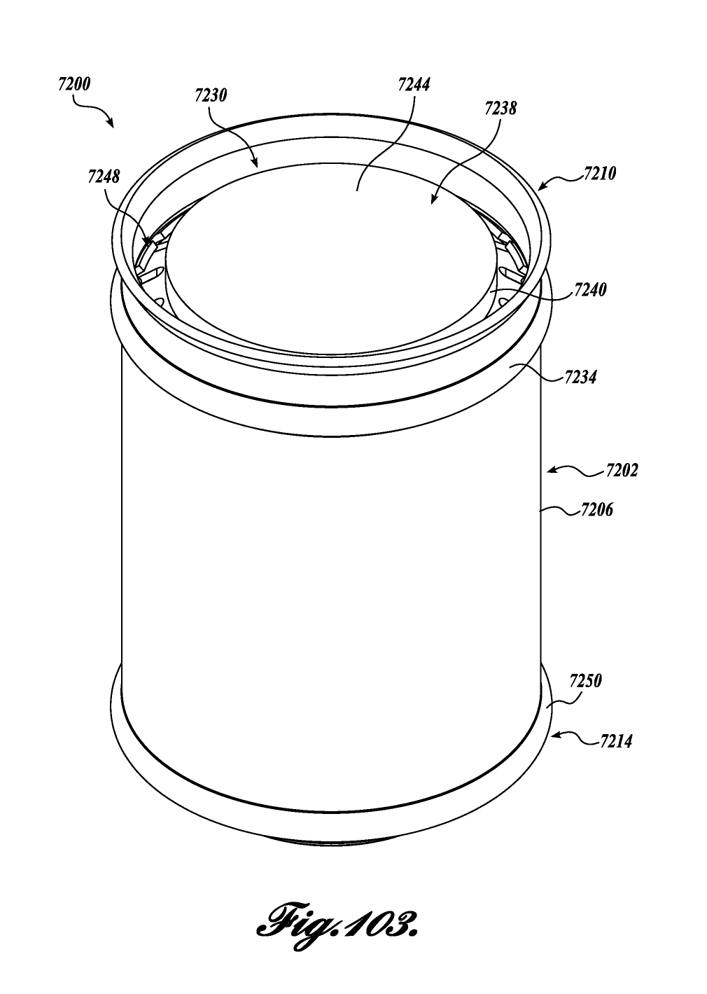

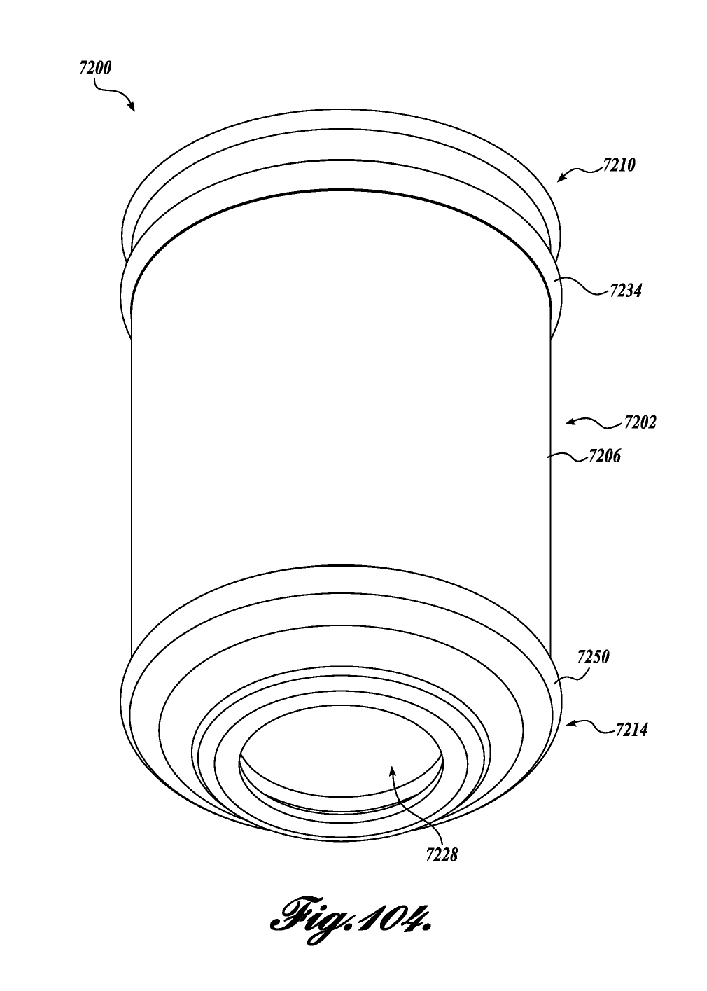

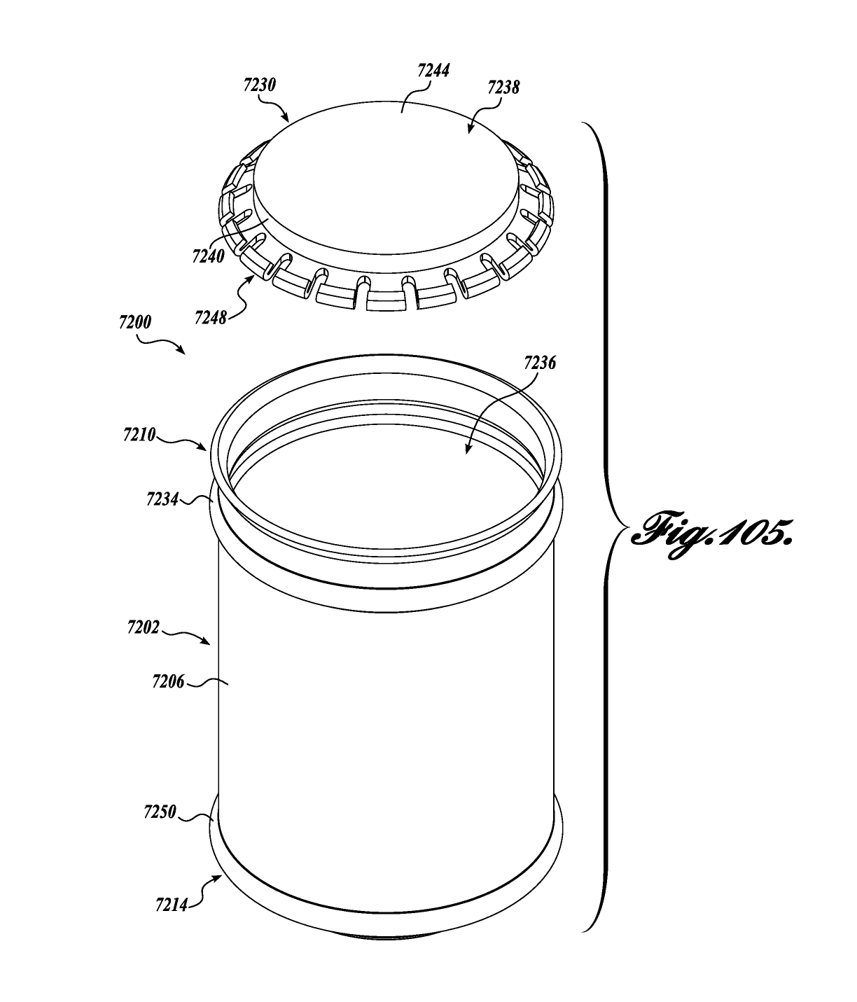

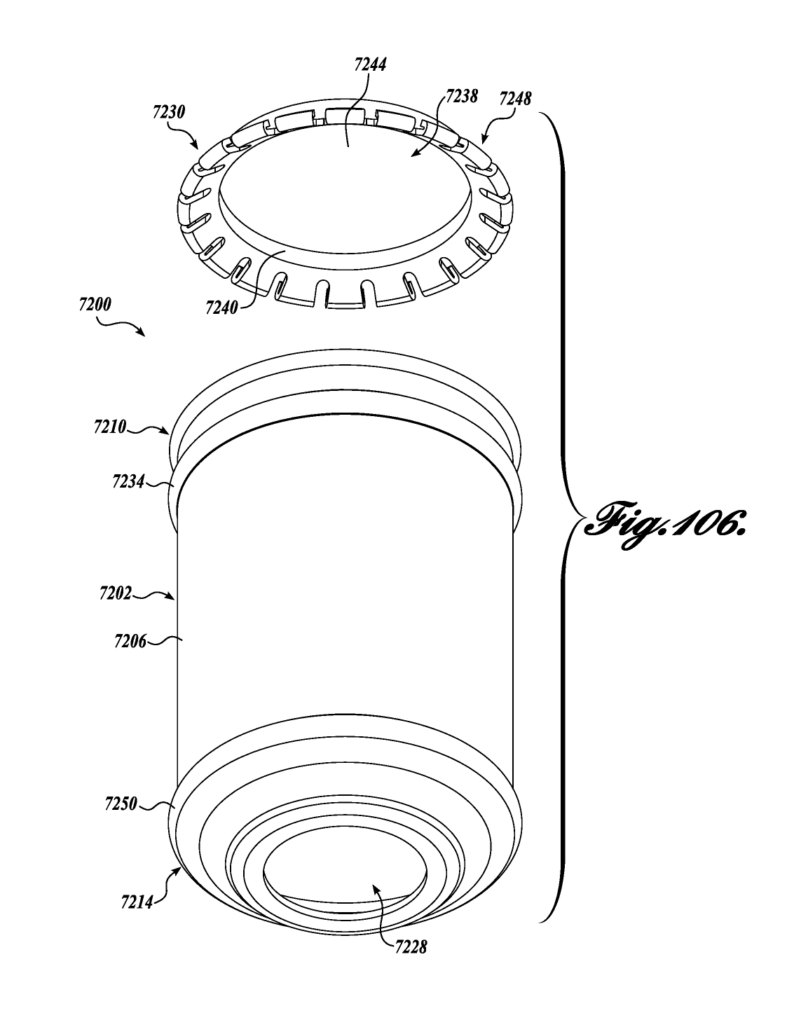

Referring to FIGS. 103-108, another exemplary embodiment of a carrier 7200 formed in accordance with the present disclosure is shown. The carrier 7200 is illustrated as composed of a body 7202 having a length longer than the container Cm, which is supported and carried by the body 7202. The body 7202 includes a cylindrical portion 7206 extending along a majority of the length of the body 7202, a first end portion 7210 defined at a first end of the cylindrical portion 7206, and a second end portion 7214 defined at a second end of the cylindrical portion 7206 opposite the first end portion 7210. First and second, or upper and lower openings 7236 and 7228, are defined in the first and second ends of the cylindrical portion 7206 for facilitating heat transfer with the contents of the container Cm.

The cylindrical portion 7206 has a generally cylindrical exterior and interior, and an inside diameter larger than the container Cm. More specifically, the interior of the body 7102 is shown as being cylindrical in shape and sized to receive the container C′″ with clearance between the exterior of the container and the interior of the body. Of course, the interior of the body can be other than cylindrical in shape, especially if the body is molded from plastic or other moldable or formable material. However, if the body is fabricated from cylindrical material stock, then the interior of the body can simply match the exterior. Regardless, the body 7102 can be formed from a durable material, such as a high strength plastic or metal. Moreover, as with other carriers (such as, for example, carriers 4100 and 4200), slots, holes, or other types of openings can be formed in the body 7202 to facilitate heat transfer with the contents of the container C′″.

Although not shown, the body 7202 can include rims or chines, for example, at the ends of the body, to add strength to the body as well as provide contact surfaces about which the body may roll. Also, if the exterior of the body is not cylindrical in shape, circular rims or chines provide surface about which the body can roll.

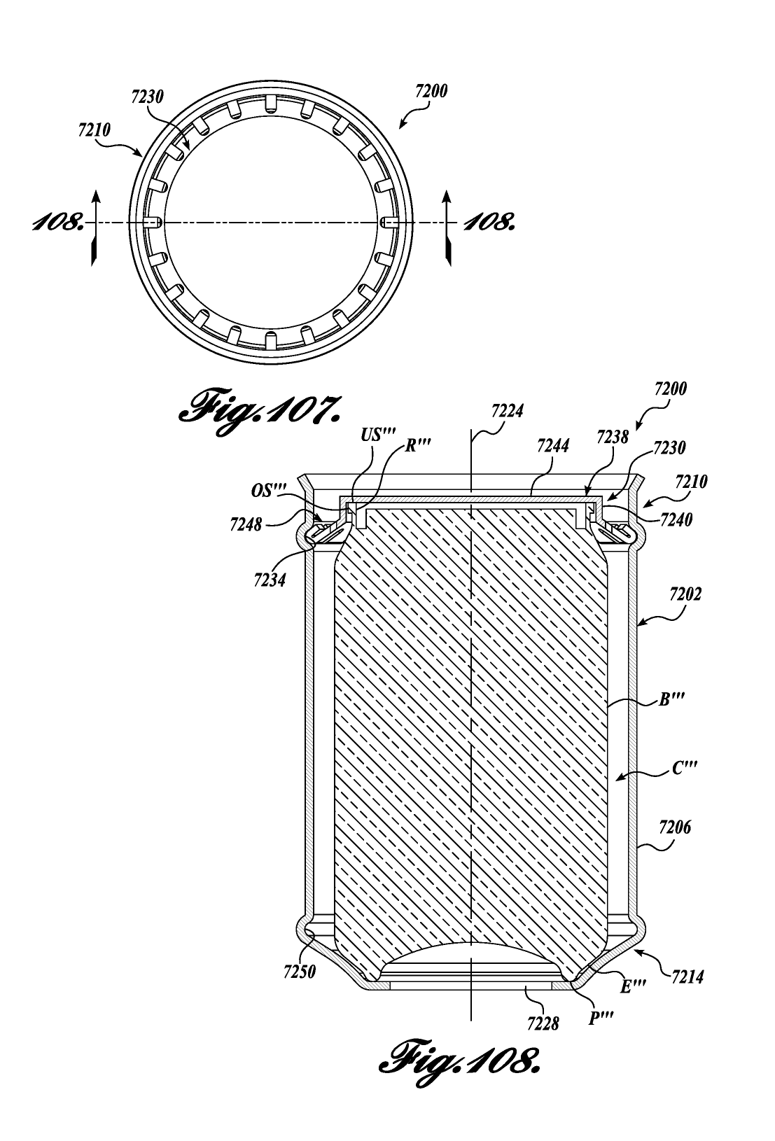

Referring to FIG. 108, the second end portion 7214 of the body 7202 will first be described in detail. The second end portion 7214 of the body 7202 is generally configured to capture and retain a second end of the container C′″ within the body 7202. In the illustrated embodiment, the container C′″ is a 2-piece can substantially identical to the 2-piece cans C, C′, and C″ shown in FIGS. 91, 97, and 102. As such, like parts have been identified with like reference letters except with a triple prime′.

The second end portion 7214 of the body 7202 is generally configured to capture and retain the container C′″ in a centrally located position (related to a central axis 7224) within the body. In that regard, the second end portion 7214 is substantially identical to the second end portion 7114 of carrier 7100. More specifically, the second end portion 7214 is shaped to receive the bottom of the container C′″ defined by the inwardly and downwardly annular sloped end surface E′″ and the annular protrusion P″. In that regard, the second end portion 7214 is generally shaped to match the shape of the bottom of the container C′″.

The first end portion 7210 of the body 7202 will now be described in detail. The first end portion 7210 is generally configured to removably receive a retaining member, such as a clip 7230, for bearing against the first end of the container C′″ when received within the carrier 7200. In that regard, the first end portion 7210 includes an interior annular groove 7234 that is concentric with the central axis 7224 and that is configured to position the clip 7230 against the first end of the container C′″. The annular groove 7234 may be any suitable cross-sectional shape and size to help ease the clip 7230 into and out of the groove 7234. The second end portion 7214 may have a substantially identical annular groove 7250 that defines an exterior rim to help facilitate rolling of the carrier 7200 on the exterior of the annular grooves 7234 and 7250.

The clip 7230, which can best be seen in FIGS. 105-107, has a clip body 7238 that is generally cylindrical in shape and is sized and configured to receive the upper annular rim R′″ of the container C′″ therein. In that regard, the clip body 7238 has a cylindrical wall section 7240 that is configured to engage or be positioned near the outer surface OS′″ of the annular rim R′″. The clip body 7238 further includes a transverse end section 7244 enclosing the cylindrical wall section 7240 that is configured to engage the upper surface US′″ of the annular rim R′″.

The clip 7230 also includes a plurality of radial projections 7248 extending from the clip body 7238 downwardly away from the center of the clip. The radial projections 7248 are configured to engage the interior annular groove 7234 of the first end portion 7210 to removably secure the clip 7230 therein. In that regard, the radial projections 7248 are deformable toward one another to temporarily decrease the overall diameter of the clip 7230 for easing the clip into and out of the interior annular groove 7234. In a non-deformed state, the overall diameter of the clip 7230 is larger than the diameter of the interior annular groove 7234 to secure the clip therein. Each radial projection 7248 may have a turned up or otherwise contoured distal edge to help ease the clip 7230 into and out of the annular groove 7234.

In the depicted embodiment, the clip 7230 includes twenty radial projections 7248 spaced substantially evenly around the circumference of the clip body 7238. However, any suitable number of radial projections 7248 and any suitable spacing between projections may be used.

The radial projections 7248, or optionally the entire clip 7230, is made from an elastic material having a suitable yield strength such that it may be deformed (decreased in diameter) for moving into and out of the annular groove 7234 and released back toward its original state for being retained within the annular groove 7234. Any suitable material may be used, such as spring steel. It should be appreciated that the clip 7230 can instead be constructed of other materials that provide other characteristics similar to spring steel, for example, a flexible plastic-based material. Moreover, some or all portions of the clip 7230 can be coated with a non-abrasive material, such as rubber, to help prevent any scratching or other damage to the container C′″ when engaged by the clip 7230.

In use, the clip 7230 is secured within the annular groove 7234 after the container C′″ is disposed within the body 7202 such that the bottom of the container, and specifically, the annular sloped end surface E′″ and the annular protrusion P″ are engaged with the interior of the second end portion 7214. With the container C′″ secured within the carrier 7200 in this manner, the clip 7230 may be disposed within the annular groove 7234 such that the clip body 7238 is positioned against the annular rim R′″. The clip 7230 therefore secures the container C′ within the body 7202 of the carrier 7200 and prevents the container from moving within the carrier during processing.

Moreover, as with other carriers, the body 7202 is sufficiently large in diameter, and the container C′″ is positioned within the carrier such that the sides of the container C′″ are not touched by the body 7202. Accordingly, the carrier 7200 is suitable for transporting a container through a sterilizer while minimizing any scratching or other damage to the sides of the container during processing.

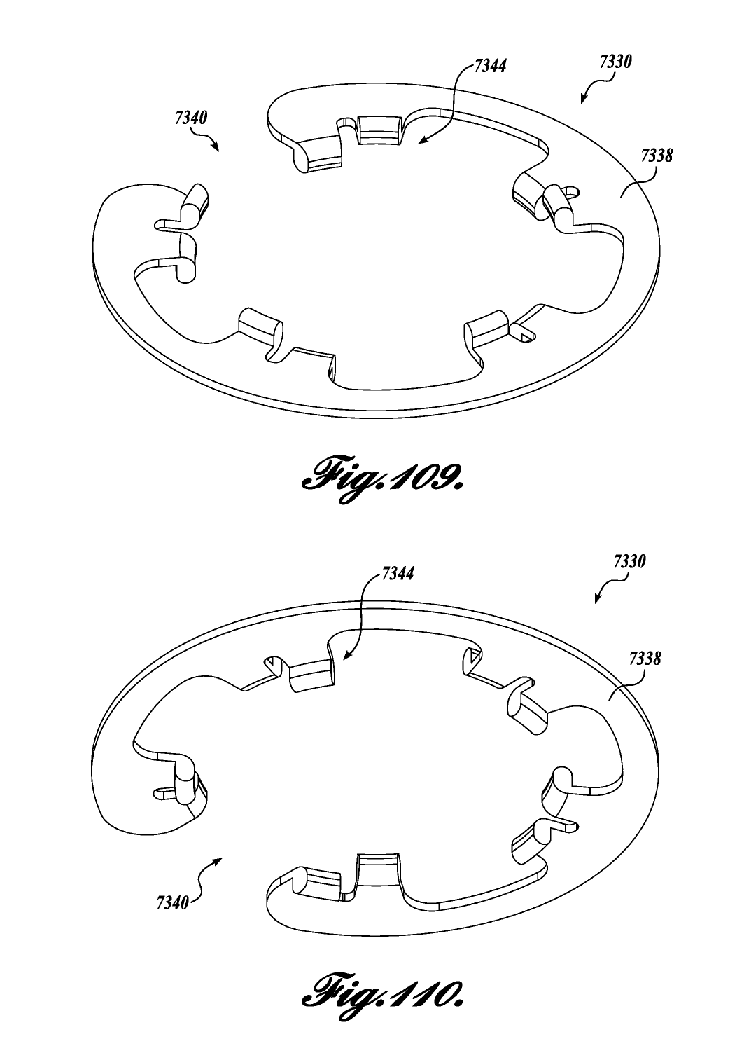

Referring to FIGS. 109-112, first and second exemplary alternative embodiments of clips 7330 and 7430 are depicted. The clips 7330 and 7430 may be used with any of the above-described carriers 7000, 7100, and 7200, modified as needed, or with any other suitable carrier. Clips 7330 and 7430 each include a clip body 7338 and 7438, respectively, that is generally C-shaped and has a circular outer diameter that is somewhat larger in an undeformed state than the interior diameter of the annular groove, body, etc., in which it is removably received.

With the clip bodies 7338 and 7438 being C-shaped, the bodies each include a gap 7340 and 7440, respectively, that may be decreased or increased in size to decrease or increase the size of the outer diameter of the clip body. When received within an annular groove, body, etc., the clip may be released back toward its original shape and size.

The clips 7330 and 7430 are made from an elastic material having a suitable yield strength such that it may be deformed (decreased in diameter) for moving into and out of an annular groove, body, etc., and released back toward its original state for being retained therein. Any suitable material may be used, such as spring steel. It should be appreciated that the clips 7330 and 7430 can instead be constructed of other materials that provide other characteristics similar to spring steel, for example, a flexible plastic-based material. Moreover, some or all portions of the clips 7330 and 7430 can be coated with a non-abrasive material, such as rubber, to help prevent any scratching or other damage to the container when engaged by the clip.

Each clip 7330 and 7430 includes a plurality of radial projections 7344 and 7444, respectively, extending from the clip body 7338 and 7438, respectively, toward the center of the clip. The radial projections 7344 and 7444 extend upwardly and downwardly from the clip bodies 7338 and 7438, respectively, in an alternating fashion. The radial projections 7344 and 7444 are also of a suitable length and contour to appropriately engage a desired first or second end of a container. For instance, one set of the upwardly or downwardly extending radial projections 7344 and 7444 may be configured to hook around and engage the annular rim R of a container C or the annular protrusion P of a container C. In that regard, the radial projections 7344 and 7444 may be suitably contoured to engage the container without substantially scratching or damaging the container during processing. The other set of the upwardly or downwardly extending radial projections 7344 and 7444 may be configured to be grasped by a user (or with a suitable tool) to deform the clips 7330 and 7430, respectively, for moving into and out of engagement with the carrier.

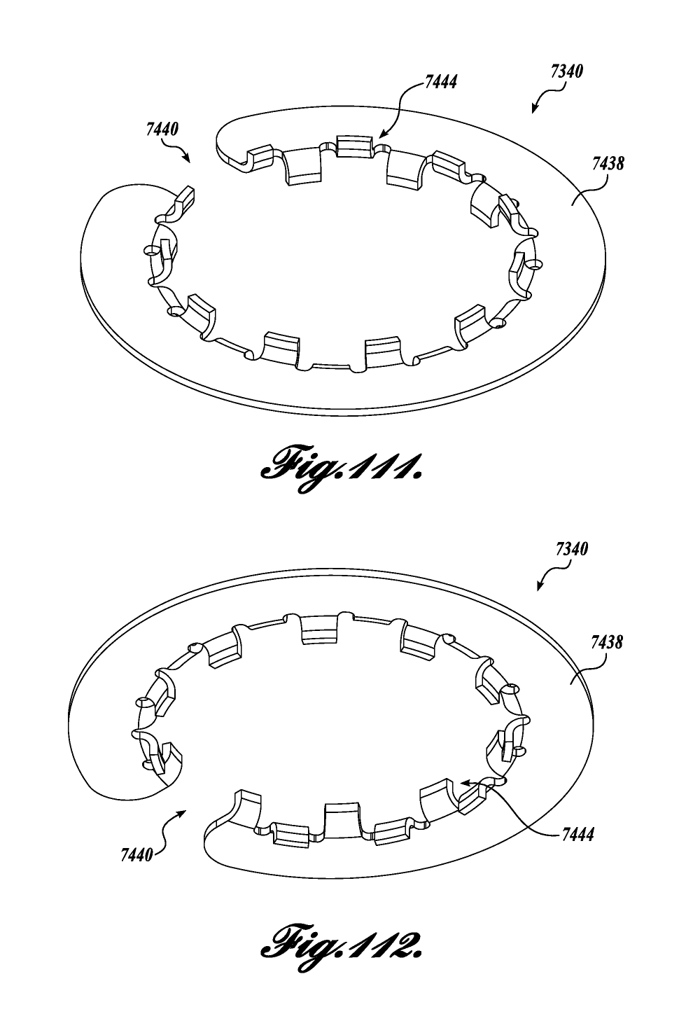

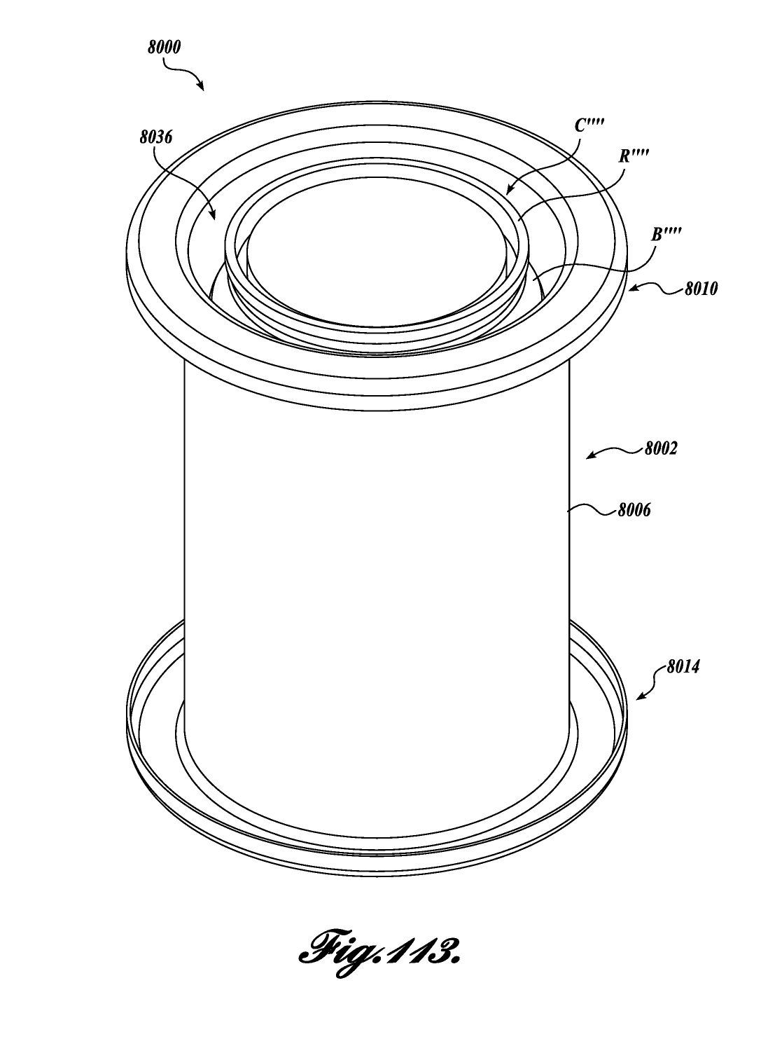

In the depicted embodiment of FIGS. 109 and 110, the clip 7330 includes five sets of alternating upwardly and downwardly extending radial projections 7344 spaced substantially evenly around the circumference of the clip body 7338. In the depicted embodiment of FIGS. 111 and 112, the clip 7430 includes twenty-one alternating upwardly and downwardly extending radial projections 7444 extending around the circumference of the clip body 7438. However, any suitable number of radial projections and any suitable spacing between projections may be used.

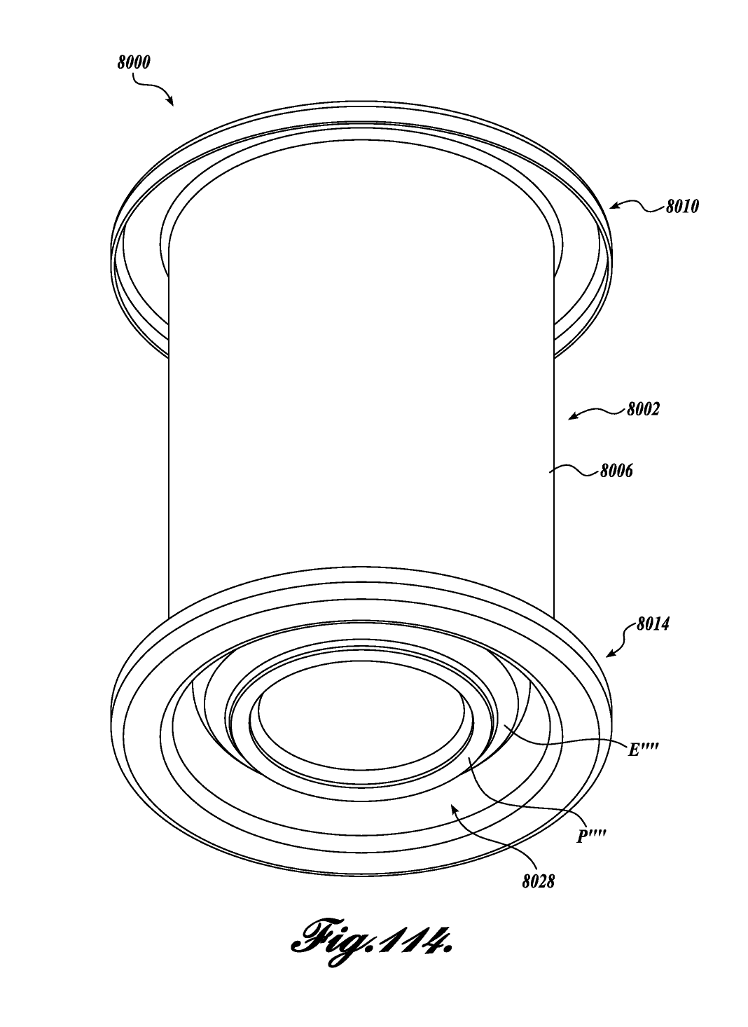

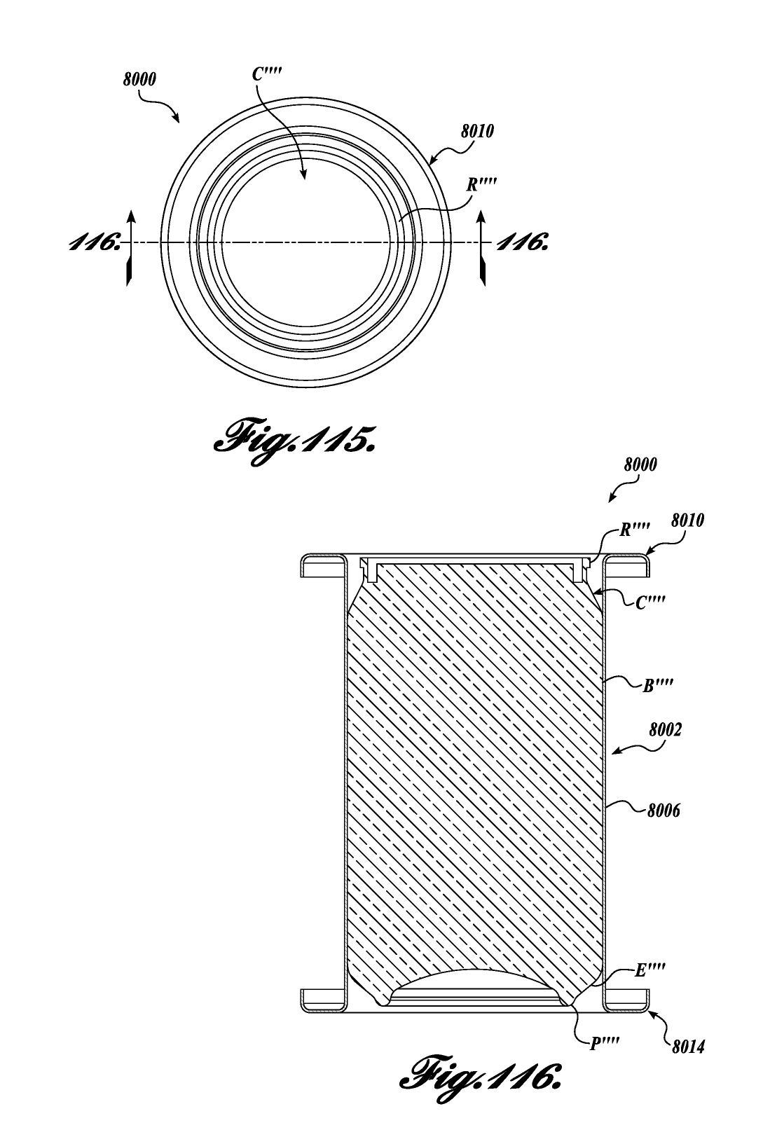

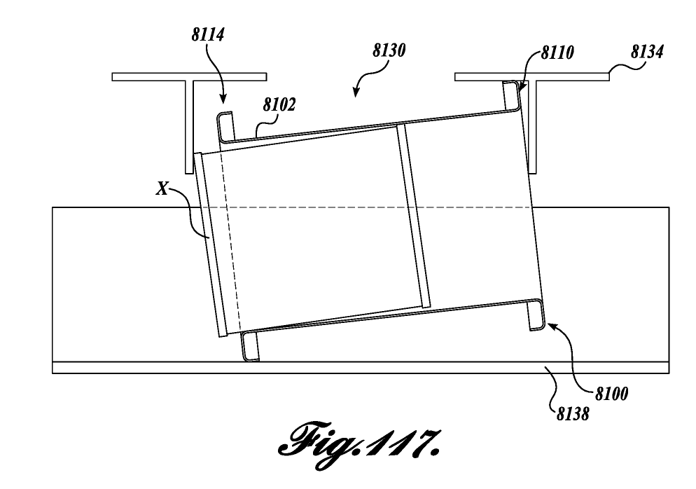

Referring to FIGS. 113-116, another exemplary embodiment of a carrier 8000 formed in accordance with the present disclosure is shown. Unlike many of the carriers described above, in one embodiment, carrier 8000 is configured to tightly engage and bear against the sides of a container for moving the container in the sterilizer for processing. As can be appreciated, the carrier 8000 is configured for use with non-lithographed containers or containers that need not remain completely undamaged or untouched on its sides. In the depicted embodiment, the carrier 8000 is generally configured to hold a container C″″ embodied as a 2-piece can substantially identical to the 2-piece cans C, C′, and C″ shown in FIGS. 91, 97, and 102. As such, like parts have been identified with like reference letters except with a quadruple prime″″.

The carrier 8000 is illustrated as composed of a body 8002 having a length longer than the container C″, which is supported and carried by the body 8002. In some embodiments, the carrier 8000 may be configured to carry more than one container. In such an alternative embodiment, the body 8002 may be longer than the total combined length of the plurality of containers. The carrier 8000 may instead be arranged to carry multiple containers in any other suitable manner.

The body 8002 includes a cylindrical portion 8006 extending along a majority of the length of the body 8002, a first end portion 8010 defined at a first end of the cylindrical portion 8006, and a second end portion 8014 defined at a second end of the cylindrical portion 8006 opposite the first end portion 8010. First and second, or upper and lower openings 8036 and 8028 are defined in the first and second ends of the cylindrical portion 8006 for facilitating heat transfer with the contents of the container C″.

In the embodiment depicted in FIGS. 113-116, the cylindrical portion 8006 has a generally cylindrical exterior and interior, and an inside diameter that is substantially the same as the outside diameter of the container C″. More specifically, the interior of the body 8002 is shown as being cylindrical in shape and sized to receive the container C″″ with substantially little to no diametric clearance between the exterior of the container and the interior of the body. In a specific example, the diametric clearance between the exterior of the container and the interior of the body is about 0.06 inches to about 0.12 inches. In another example, however, the diametric clearance between the exterior of the container and the interior of the body can be up to about 0.50 inches, as later described with reference to FIG. 117. Of course, any suitable diametrical clearance for suitably holding and carrying the container in the rotary processor may be used.

The interior of the body can be other than cylindrical in shape to match the shape of the container, especially if the body is molded from plastic or another moldable or formable material. However, if the body is fabricated from cylindrical material stock, then the interior of the body can simply match the exterior. Regardless, the body 8002 can be formed from a durable material, such as a high-strength plastic or metal. Moreover, as with other carriers (such as, for example, carriers 4100 and 4200), slots, holes, or other types of openings can be formed in the body 8002 to facilitate heat transfer with the contents of the container C″.

The first and second end portions 8010 and 8014 are substantially identical; and therefore, only the first end portion 8010 will be described in detail. The first end portion 8010 is generally defined by an annular folded over rim extending radially outwardly from the upper or first end of the cylindrical portion 8006 of the body 8002. The annular folded over rim is generally U-shaped in cross-section, with the bottom portion of the U-shaped annular rim substantially transverse to the side portions and with curved junctures therebetween. The annular folded over rim of the first end portion 8010 provides a supportive cylindrical structure on which the carrier 8000 may roll. More specifically, the folded-over rim of the first end portion 8010 helps provide axial and radial impact resistance to the first end of the carrier 8000 as it travels through the sterilizer.

The second end portion 8014 includes an annular folded over rim that is substantially the same shape and size as the annular folded over rim of the first end portion 8010. In this manner, the carrier 8000 may roll evenly on the first and second end portions 8010 and 8014 with the cylindrical portion 8006 of the body 8002 remaining out of contact with any rolling surface. Also, if the exterior of the body 8002 is not cylindrical in shape, the circular first and second end portions 8010 and 8014 provide surface about which the body can roll. It should be appreciated that the first and second end portions 8010 and 8014 may instead be any other suitable shape or configuration to provide a suitable rolling surface for the carrier 8000.

Referring to FIG. 117, another exemplary embodiment of a carrier 8100 formed in accordance with the present disclosure is shown. The carrier 8100 is substantially identical to the carrier 8000 described above, having a body 8102 extending between first and second end portions 8110 and 8114. However, the body 8102 is larger in diameter than body 8002 to define a larger diametric clearance between the exterior of the container X and the interior of the body 8102. In other words, a gap is defined between the container X and the interior of the body 8102. As can be appreciated, a container having a smaller outer diameter compared to container C″, such as the container X depicted, may instead be merely used with carrier 8000.

The diametric clearance between the exterior of the container X and the interior of the body 8102 may be up to about 0.50 inches, or any other suitable amount. With a gap defined between the exterior of the container X and the interior of the body 8102, the container X will necessarily shift within the carrier 8100 during processing. Accordingly, such a carrier embodiment is well suited for a rotary processor that defines a suitable pocket for retaining the container X within the carrier 8100 during processing.

In the embodiment depicted in FIG. 117, the processor includes a rotary pocket 8130 created by the interface of a spiral tee 8134 and an angle 8138 on the reel (see also FIG. 18). The pocket 8130 is sized to retain the container X and carrier 8100 on the reel during processing, even if the container X is loosely disposed within the carrier 8100. It should be appreciated that the pocket may instead be defined in any other suitable manner.

The shapes and constructions of the carriers described and illustrated herein are only representative examples of the shapes that the carriers may take, in accordance with the present disclosure.

Moreover, it should be appreciated that reference to a first end, second end, upper end, bottom end, etc., of the container and/or the carrier or carrier body is for illustrative purposes only, and should not be seen as limiting. For example, any of the foregoing carriers may be modified to receive and hold a container in a manner inverted from what is shown.

In addition, the embodiments of the present disclosure have been described for use with rotary processing systems. It is to be appreciated that the carriers of the present disclosure also can be used with other types of thermal processing systems, including retorts having drums that may or may not rotate during the processing of food products or other non-food-related rotary processes.

Moreover, numerous of the embodiments of the present disclosure have been described and illustrated for use in conjunction with a container C or the like, in the form of a cylindrical can. However, the carriers of the present disclosure can be used in conjunction with numerous other types of containers, including cans or bottles or boxes that are cylindrical, spherical or polygonal in cross-sectional shape or even with containers in the form of pouches or envelopes.

Furthermore, numerous of the embodiments of the present disclosure have been described and illustrated for use with a single container. However, it should be appreciated that the carriers of the present disclosure may be adapted for carrying two or more containers (for instance, with the containers stacked upon one another).

1. A carrier for processing a container in a thermal processing system, the container having a container body extending between first and second container ends, the carrier comprising:
a carrier body having an exterior configured to roll in the thermal processing system, the carrier body having a first end portion configured to receive the first container end and a second end portion opposite the first end portion; and
a retention system disposed within the second end portion that is configured to engage the second container end and retain the container within the carrier body.

2. The carrier of claim 1, wherein the carrier body has an interior diameter larger than an exterior diameter of the container body such that the carrier engages the container at only the first and second container ends.

3. The carrier of claim 1, wherein the retention system is defined by a retaining member removably received within the second end portion of the carrier body, the retaining member engageable with the second container end for retaining the container in place within the carrier body.

4. The carrier of claim 3, wherein the retaining member is removably receivable within an annular groove defined in the second end portion of the carrier body.

5. The carrier of claim 3, wherein the retaining member is C-shaped.

6. (canceled)

7. The carrier of claim 3, wherein the retaining member is deformable for moving into and out of engagement with the second end portion of the body.

8. (canceled)

9. The carrier of claim 1, wherein the container is a cylindrical can having an annular rim at a first end and an annular protrusion at a second end.

10. The carrier of claim 9, wherein the retention system is engageable with the annular protrusion at the second container end.

11. The carrier of claim 10, wherein an interior of the first end portion of the carrier body is configured to engage a corner of the annular rim.

12. The carrier of claim 10, wherein the retaining member is C-shaped.

13. The carrier of claim 1, wherein the retention system is defined by at least one flexible finger configured to receive the container and hold the container within the carrier body, wherein the at least one finger is moveable between a first position, wherein the at least one finger is spaced from the container to receive the container into the carrier body, and a second position, wherein the at least one flexible finger holds the container within the carrier body.

14-21. (canceled)

22. A carrier for a container for processing the container in a thermal processing system, the carrier comprising:
a body having an exterior configured to roll in a thermal processing system, the body having a first end portion configured to receive a first end of a container and a second end portion opposite the first end portion; and
a retaining member removably received within the second end portion of the body, the retaining member engageable with a second end of the container for retaining the container in place within the body.

23. The carrier of claim 22, wherein the body has an interior with a diameter larger than an exterior diameter of the container.

24. The carrier of claim 22, wherein the retaining member is removably receivable within an annular groove defined in the second end portion of the body.

25. The carrier of claim 22, wherein the retaining member is C-shaped.

26-28. (canceled)

29. The carrier of claim 22, wherein the container is a cylindrical can having an annular rim at a first end and an annular protrusion at a second end.

30. The carrier of claim 29, wherein the retaining member is engageable with the annular protrusion at the second end of the container.

31. The carrier of claim 30, wherein an interior of the first end portion of the body is configured to engage a corner of the annular rim.

32. The carrier of claim 30, wherein the retaining member is C-shaped.

33-46. (canceled)

47. A rotary processing system, comprising:
a pocket configured to receive and move a carrier within the rotary processing system; and
a carrier sized to be received and retained within the pocket during movement within the rotary processing system, the carrier comprising a body having first and second end portions that are configured to roll in the thermal processing system, the body having an interior configured to receive a container, wherein the interior of the body has a diameter that is larger than the outer diameter of the container.

48-51. (canceled)
